(19) United States
(12) Patent Application Publication
(10) Pub. No.: US 2024/0000970 A1
(43) Pub. Date: Jan. 4, 2024

(54) DIFFERENTIAL KNOCKOUT OF A HETEROZYGOUS ALLELE OF LRRK2

(71) Applicant: EmendoBio Inc., Wilmington, DE (US)

(72) Inventors: Rafi Emmanuel, Ramla (IL); Michal Golan Mashiach, Ness Ziona (IL)

(73) Assignee: EmendoBio Inc., Wilmington, DE (US)

(21) Appl. No.: 18/255,264

(22) PCT Filed: Dec. 1, 2021
(86) PCT No.: PCT/US2021/061334

§ 371 Date: May 31, 2023

Related U.S. Application Data
(60) Provisional application No. 63/213,507, filed on Jun. 22, 2021.
(60) Provisional application No. 63/119,905, filed on Dec. 1, 2020.

(51) Int. Cl. A61K 48/00; C12N 9/22; C12N 15/113; A61P 25/16; C12N 5/079

(52) U.S. Cl. CPC A61K 48/005; C12N 9/22; C12N 15/1137; A61P 25/16; C12N 5/0618

(57) ABSTRACT

RNA molecules comprising a guide sequence portion having 17-50 contiguous nucleotides in the sequence set forth in any one of SEQ ID NOs: 1-103,606 and compositions, methods, and uses thereof.

This application claims the benefit of U.S. Provisional Application Nos. 63/213,506, filed Jun. 22, 2021, and 63/119,905, filed Dec. 1, 2020, the contents of which are hereby incorporated by reference.

Throughout this application, various publications are referenced, including referenced in parenthesis. The disclosures of all publications mentioned in this application in their entireties are hereby incorporated by reference into this application in order to provide additional description of the art to which this invention pertains and of the features in the art which can be employed with this invention.

### REFERENCE TO SEQUENCE LISTING

This application incorporates-by-reference nucleotide sequences which are present in the file named “211201_91648-A-PCT_Sequence_Listing_AWG.txt”, which is 19,634 kilobytes in size, and which was created on Nov. 29, 2021 in the IBM-PC machine format, having an operating system compatibility with MS-Windows, which is contained in the text file filed Dec. 1, 2021 as part of this application.

### BACKGROUND OF INVENTION

There are several classes of DNA variation in the human genome, including insertions and deletions, differences in the copy number of repeated sequences, and single nucleotide polymorphisms (SNPs). A SNP is a DNA sequence variation occurring when a single nucleotide (adenine (A), thymine (T), cytosine (C), or guanine (G)) in the genome differs between human subjects or paired chromosomes in an individual. Over the years, the different types of DNA variations have been the focus of the research community either as markers in studies to pinpoint traits or disease causation or as potential causes of genetic disorders.

A genetic disorder is caused by one or more abnormalities in the genome. Genetic disorders may be regarded as either “dominant” or “recessive.” Recessive genetic disorders are those which require two copies (i.e., two alleles) of the abnormal/defective gene to be present. In contrast, a dominant genetic disorder involves a gene or genes which exhibit(s) dominance over a normal (functional/healthy) gene or genes. As such, in dominant genetic disorders only a single copy (i.e., allele) of an abnormal gene is required to cause or contribute to the symptoms of a particular genetic disorder. Such mutations include, for example, gain-of-function mutations in which the altered gene product possesses a new molecular function or a new pattern of gene expression. Other examples include dominant negative mutations, which have a gene product that acts antagonistically to the wild-type allele.

### Leucine-Rich Repeat Serine/Threonine-Protein Kinase 2 (LRRK2) and Monogenic Parkinson's Disease (PD)

Leucine-rich repeat serine/threonine-protein kinase 2 (LRRK2) is a generalist kinase that phosphorylates a broad range of proteins involved in multiple processes, such as neuronal plasticity, autophagy, and vesicle trafficking. It is a key regulator of Rab GTPases such as the Rab-3 family (Rab-3A, Rab-3B, Rab-3C, Rab-3D), the Rab-5 family (Rab-5A, Rab-5B, Rab-5C), the Rab-8 family (Rab-8A, Rab-8B), Rab-10, Rab-12, Rab-35, and Rab-43. It also regulates their interaction partners by extension in modulating the exchange between GDP and GTP on these proteins. These interacting partners govern a wide variety of cellular processes, including autophagy, cell differentiation, cell transport, synaptic vesicle trafficking, retrograde trafficking pathways for protein recycling, neuronal morphology in the central nervous system (CNS), and inhibiting degradation of the microtubule-associated protein tau (MAPT).

The LRRK2 G2019S mutation (p.Gly2019Ser, c.6055G>A) is the most common PD-associated mutation known. Indeed, LRRK2 G2019S was reported in 41% of sporadic and 37% of familial PD patients (compared to 3% in healthy controls) from the North African Arab population, and in 18.3% of Ashkenazi Jewish PD patients (compared to 1.3% in controls).

While the precise pathogenic mechanism is unknown, the characterized mutations (such as Gly2019Ser in exon 41) appear to disrupt kinase or GTPase activity. These disruptions in activity seem to result in increased intra- and intermolecular phosphorylation of known LRRK2 substrates. LRRK2-mediated PD is similar to idiopathic PD, which is a disease characterized by progressive degeneration of motor functions, progressive asymmetric tremors at rest, muscle rigidity, postural instability, REM sleep behavior disorders, cognitive decline, and gait abnormalities. Some of these symptoms are less severe in LRRK2-mediated PD patients than they are for idiopathic PD patients. Onset for LRRK2-mediated PD is typically after age 50, though early-onset (20s) and late-onset (90s) versions of the disease have been described. The LRRK2 gene consists of 51 exons, with the coding region starting on exon 1, and the G1y2019Ser mutation occurs in exon 41, in what is known as the “activation hinge” of the kinase domain.

### SUMMARY OF THE INVENTION

Disclosed is an approach for knocking out the expression of a dominant-mutated LRRK2 allele by disrupting the dominant-mutated allele or degrading the resulting mRNA.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (REF/SNP), and the other allele encoding for a functional protein (“functional allele”). In some embodiments, the SNP position is utilized for distinguishing/discriminating between two alleles of a gene bearing one or more disease associated mutations, such as to target one of the alleles bearing both the particular sequence in the SNP position (SNP/REF) and a disease associated mutation. In some embodiments, the disease-associated mutation is targeted. In some embodiments, the method further comprises the step of knocking out expression of the mutated protein and allowing expression of the functional protein.

The present disclosure also provides a method for modifying in a cell a mutant allele of the Leucine-rich repeat serine/threonine-protein kinase 2 (LRRK2) gene having a mutation associated with monogenic Parkinson's disease, the method comprising
introducing to the cell a composition comprising:
   a CRISPR nuclease or a sequence encoding the CRISPR nuclease; and
   a first RNA molecule comprising a guide sequence portion having 17-50 nucleotides or a nucleotide sequence encoding the same,
wherein a complex of the CRISPR nuclease and the first RNA molecule affects a double strand break in the mutant allele of the LRRK2 gene.

According to embodiments of the present invention, there is provided a first RNA molecule comprising a guide sequence portion having 17-50 contiguous nucleotides containing nucleotides in the sequence set forth in any one of SEQ ID Nos: 1-103,606.

According to some embodiments of the present invention, there is provided a composition comprising an RNA molecule comprising a guide sequence portion having 17-50 contiguous nucleotides containing nucleotides in the sequence set forth in any one of SEQ ID NOs: 1-103,606 and a CRISPR nuclease.

According to some embodiments of the present invention, there is provided a method for inactivating a mutant LRRK2 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-103,606 and a CRISPR nuclease. In some embodiments, the cell is a stem cell. In some embodiments, the cell is a neuron. In some embodiments, the cell is a dopaminergic cell. In some embodiments, the delivering to the cell is performed in vitro, ex vivo, or in vivo. In some embodiments, the method is performed ex vivo and the cell is provided/explanted from an individual patient. In some embodiments, the method further comprises the step of introducing the resulting cell, with the modified/knocked out mutant LRRK2 allele, into the individual patient (e.g., autologous transplantation).

According to some embodiments of the present invention, there is provided a method for treating monogenic Parkinson's disease, the method comprising delivering to a cell of a subject having monogenic Parkinson's disease a composition comprising an RNA molecule comprising a guide sequence portion having 17-50 contiguous nucleotides containing nucleotides in the sequence set forth in any one of SEQ ID NOs: 1-103,606 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-103,606 and a CRISPR nuclease for inactivating a mutant LRRK2 allele in a cell, comprising delivering to the cell the composition comprising an RNA molecule comprising a guide sequence portion having 17-50 contiguous nucleotides containing nucleotides in the sequence set forth in any one of SEQ ID NOs: 1-103,606 and a CRISPR nuclease.

According to embodiments of the present invention, there is provided a medicament comprising an RNA molecule comprising a guide sequence portion having 17-50 contiguous nucleotides containing nucleotides in the sequence set forth in any one of SEQ ID NOs: 1-103,606 and a CRISPR nuclease for use in inactivating a mutant LRRK2 allele in a cell, wherein the medicament is administered by delivering to the cell the composition comprising an RNA molecule comprising a guide sequence portion having 17-50 contiguous nucleotides containing nucleotides in the sequence set forth in any one of SEQ ID NOs: 1-103,606 and a CRISPR nuclease.

According to some embodiments of the present invention, there is provided use of a composition comprising an RNA molecule comprising a guide sequence portion having 17-50 contiguous nucleotides containing nucleotides in the sequence set forth in any one of SEQ ID NOs: 1-103,606 and a CRISPR nuclease for treating ameliorating or preventing monogenic Parkinson's disease, comprising delivering to a cell of a subject having or at risk of having monogenic Parkinson's disease the composition of comprising an RNA molecule comprising a guide sequence portion having 17-50 contiguous nucleotides containing nucleotides in the sequence set forth in any one of SEQ ID NOs: 1-103,606 and a CRISPR nuclease. In some embodiments, the method is performed ex vivo and the cell is provided/explanted from the subject. In some embodiments, the method further comprises the step of introducing the resulting cell, with the modified/knocked out mutant LRRK2 allele, into the subject (e.g. autologous transplantation).

According to some embodiments of the present invention, there is provided a medicament comprising the composition comprising an RNA molecule comprising a guide sequence portion having 17-50 contiguous nucleotides containing nucleotides in the sequence set forth in any one of SEQ ID NOs: 1-103,606 and a CRISPR nuclease for use in treating ameliorating or preventing monogenic Parkinson's disease, wherein the medicament is administered by delivering to a cell of a subject having or at risk of having monogenic Parkinson's disease the composition comprising an RNA molecule comprising a guide sequence portion having 17-50 contiguous nucleotides containing nucleotides in the sequence set forth in any one of SEQ ID NOs: 1-103,606 and a CRISPR nuclease.

According to some embodiments of the present invention, there is provided a kit for inactivating a mutant LRRK2 allele in a cell, comprising an RNA molecule comprising a guide sequence portion having 17-50 contiguous nucleotides containing nucleotides in the sequence set forth in any one of SEQ ID NOs: 1-103,606, a CRISPR nuclease, and/or a tracrRNA molecule; and instructions for delivering the RNA molecule; CRISPR nuclease, and/or the tracrRNA to the cell.

According to some embodiments of the present invention, there is provided a kit for treating monogenic Parkinson's disease in a subject, comprising an RNA molecule comprising a guide sequence portion having 17-50 contiguous nucleotides containing nucleotides in the sequence set forth in any one of SEQ ID NOs: 1-103,606, a CRISPR nuclease, and/or a tracrRNA molecule; and instructions for delivering the RNA molecule; CRISPR nuclease, and/or the tracrRNA to a cell of a subject having or at risk of having monogenic Parkinson's disease.

### BRIEF DESCRIPTION OF THE DRAWINGS

FIG. 1: Editing activity of guides targeting LRRK2 in A549 cells. RNPs complexed with SpCas9 CRISPR nuclease and a guide molecule comprising a specific guide sequence portion were electroporated into A549 cells to determine their activity. Cells were harvested 72 hours post RNP electroporation, genomic was DNA extracted, and the region of the mutation was amplified and analyzed by next-generation sequencing (NGS). The graph represents the % of editing±standard deviation of two (2) independent electroporations. The accompanying table below the graph indicates the genotype of A549 cells at each SNP position.

FIG. 2: Relative amount of LRRK2 RNA. A549 cells were harvested seven (7) days post-electroporation, total RNA was extracted, and the extracted RNA was subjected to reverse transcription. The level of the generated cDNA was quantified using qRT-PCR relative to untreated cells that were not edited and normalized to an endogenous gene (GAPDH). The graph represents the % of editing±standard deviation of two (2) independent electroporations.

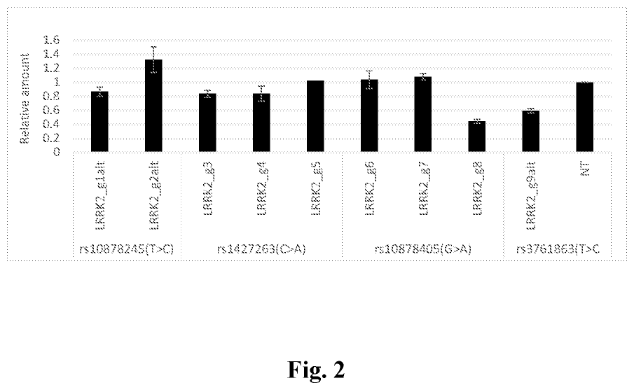

FIG. 3: Editing activity of guides targeting LRRK2 in HeLa cells with OMNI-159.

FIG. 4: Editing activity of guides targeting LRRK2 in HeLa cells with OMNI-79 V5570.

### DETAILED DESCRIPTION

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a,” “an” and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

Unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

In the description and claims of the present application, each of the verbs, “comprise,” “include” and “have” and conjugates thereof, are used to indicate that the object or objects of the verb are not necessarily a complete listing of components, elements or parts of the subject or subjects of the verb. Other terms as used herein are meant to be defined by their well-known meanings in the art.

The terms “nucleic acid template” and “donor”, refer to a nucleotide sequence that is inserted or copied into a genome. The nucleic acid template comprises a nucleotide sequence, e.g., of one or more nucleotides, that will be added to or will template a change in the target nucleic acid or may be used to modify the target sequence. A nucleic acid template sequence may be of any length, for example between 2 and 10,000 nucleotides in length, preferably between about 100 and 1,000 nucleotides in length, more preferably between about 200 and 500 nucleotides in length. A nucleic acid template may be a single stranded nucleic acid, a double stranded nucleic acid. In some embodiments, the nucleic acid template comprises a nucleotide sequence, e.g., of one or more nucleotides, that corresponds to wild type sequence of the target nucleic acid, e.g., of the target position. In some embodiments, the nucleic acid template comprises a nucleotide sequence, e.g., of one or more ribonucleotides, that corresponds to wild type sequence of the target nucleic acid, e.g., of the target position. In some embodiments, the nucleic acid template comprises modified nucleotides.

Insertion of an exogenous sequence (also called a “donor sequence,” donor template,” “donor molecule” or “donor”) can also be carried out. For example, a donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. A donor molecule may be any length, for example ranging from several bases e.g. 10-20 bases to multiple kilobases in length.

The donor polynucleotide can be DNA or RNA, single-stranded and/or double-stranded and can be introduced into a cell in linear or circular form. See, e.g., U.S. Patent Publication Nos. 2010/0047805; 2011/0281361; 2011/0207221; and 2019/0330620. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) and Nehls et al. (1996). Additional methods for protecting exogenous polynucleotides from degradation include, but are not limited to, addition of terminal amino group(s) and the use of modified intemucleotide linkages such as, for example, phosphorothioates, phosphoramidates, and O-methyl ribose or deoxyribose residues.

A donor sequence may be an oligonucleotide and be used for targeted alteration of an endogenous sequence. The oligonucleotide may be introduced to the cell on a vector, may be electroporated into the cell, or may be introduced via other methods known in the art. Donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLY)).

As used herein, the term “modified cells” refers to cells in which a double strand break is affected by a complex of an RNA molecule and the CRISPR nuclease as a result of hybridization with the target sequence, i.e. on-target hybridization. The term “modified cells” may further encompass cells in which a repair or correction of a mutation was affected following the double strand break.

This invention provides a modified cell or cells obtained by use of any of the methods described herein. In an embodiment these modified cell or cells are capable of giving rise to progeny cells. In an embodiment these modified cell or cells are capable of giving rise to progeny cells after engraftment. As a non-limiting example, the modified cells may be hematopoietic stem cells (HSCs), or any cell suitable for an allogenic cell transplant or autologous cell transplant. As a non-limiting example, the modified cells may be stem cells, neurons, or dopaminergic cells.

This invention also provides a composition comprising these modified cells and a pharmaceutically acceptable carrier. Also provided is an in vitro or ex vivo method of preparing this, comprising mixing the cells with the pharmaceutically acceptable carrier.

As used herein, the term “targeting sequence” or “targeting molecule” refers a nucleotide sequence or molecule comprising a nucleotide sequence that is capable of hybridizing to a specific target sequence, e.g., the targeting sequence has a nucleotide sequence which is at least partially complementary to the sequence being targeted along the length of the targeting sequence. The targeting sequence or targeting molecule may be part of an RNA molecule that can form a complex with a CRISPR nuclease, either alone or in combination with other RNA molecules, with the targeting sequence serving as the targeting portion of the CRISPR complex. When the molecule having the targeting sequence is present contemporaneously with the CRISPR nuclease, the RNA molecule, alone or in combination with an additional one or more RNA molecules (e.g. a tracrRNA molecule), is capable of targeting the CRISPR nuclease to the specific target sequence. As non-limiting example, a guide sequence portion of a CRISPR RNA molecule or single-guide RNA molecule may serve as a targeting molecule. Each possibility represents a separate embodiment. A targeting sequence can be custom designed to target any desired sequence.

The term “targets” as used herein, refers to preferentially hybridizing a targeting sequence of a targeting molecule to a nucleic acid having a targeted nucleotide sequence. It is understood that the term “targets” encompasses variable hybridization efficiencies, such that there is preferential targeting of the nucleic acid having the targeted nucleotide sequence, but unintentional off-target hybridization in addition to on-target hybridization might also occur. It is understood that where an RNA molecule targets a sequence, a complex of the RNA molecule and a CRISPR nuclease molecule targets the sequence for nuclease activity.

The “guide sequence portion” of an RNA molecule refers to a nucleotide sequence that is capable of hybridizing to a specific target DNA sequence, e.g., the guide sequence portion has a nucleotide sequence which is partially or fully complementary to the DNA sequence being targeted along the length of the guide sequence portion. In some embodiments, the guide sequence portion is 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides in length, or approximately 17-50, 17-49, 17-48, 17-47, 17-46, 17-45, 17-44, 17-43, 17-42, 17-41, 17-40, 17-39, 17-38, 17-37, 17-36, 17-35, 17-34, 17-33, 17-31, 17-30, 17-29, 17-28, 17-27, 17-26, 17-25, 17-24, 17-22, 17-21, 18-25, 18-24, 18-23, 18-22, 18-21, 19-25, 19-24, 19-23, 19-22, 19-21, 19-20, 20-22, 18-20, 20-21, 21-22, or 17-20 nucleotides in length. Preferably, the entire length of the guide sequence portion is fully complementary to the DNA sequence being targeted along the length of the guide sequence portion. The guide sequence portion may be part of an RNA molecule that can form a complex with a CRISPR nuclease with the guide sequence portion serving as the DNA targeting portion of the CRISPR complex. When the RNA molecule having the guide sequence portion is present contemporaneously with the CRISPR molecule, alone or in combination with an additional one or more RNA molecules (e.g. a tracrRNA molecule), the RNA molecule is capable of targeting the CRISPR nuclease to the specific target DNA sequence. Accordingly, a CRISPR complex can be formed by direct binding of the RNA molecule having the guide sequence portion to a CRISPR nuclease or by binding of the RNA molecule having the guide sequence portion and an additional one or more RNA molecules to the CRISPR nuclease. Each possibility represents a separate embodiment. A guide sequence portion can be custom designed to target any desired sequence. Accordingly, a molecule comprising a “guide sequence portion” is a type of targeting molecule. In some embodiments, the guide sequence portion comprises a sequence that is the same as, or differs by no more than 1, 2, 3, 4, or 5 nucleotides from, a guide sequence portion described herein, e.g., a guide sequence set forth in any of SEQ ID NOs:1-103,606. Each possibility represents a separate embodiment. In some embodiments, the guide sequence portion comprises a sequence that is the same as, or differs by up to 1, 2, 3, 4, or 5 nucleotides from, a guide sequence portion described herein, e.g., a guide sequence set forth in any of SEQ ID NOs:1-103,606. Each possibility represents a separate embodiment. In some of these embodiments, the guide sequence portion comprises a sequence that is the same as a sequence set forth in any of SEQ ID NOs:1-103,606. Throughout this application, the terms “guide molecule,” “RNA guide molecule,” “guide RNA molecule,” and “gRNA molecule” are synonymous with a molecule comprising a guide sequence portion.”

The term “non-discriminatory” as used herein refers to a guide sequence portion of an RNA molecule that targets a specific DNA sequence that is common both a mutant and functional allele of a gene.

In embodiments of the present invention, an RNA molecule comprises a guide sequence portion having 17-50 contiguous nucleotides containing nucleotides in the sequence set forth in any one of SEQ ID NOs: 1-103,606.

The RNA molecule and or the guide sequence portion of the RNA molecule may contain modified nucleotides. Exemplary modifications to nucleotides or polynucleotides may be synthetic and encompass polynucleotides which contain nucleotides comprising bases other than the naturally occurring adenine, cytosine, thymine, uracil, or guanine bases. Modifications to polynucleotides include polynucleotides which contain synthetic, non-naturally occurring nucleosides e.g., locked nucleic acids. Modifications to polynucleotides may be utilized to increase or decrease stability of an RNA. An example of a modified polynucleotide is an mRNA containing 1-methyl pseudo-uridine. For examples of modified polynucleotides and their uses, see U.S. Pat. No. 8,278,036, PCT International Publication No. WO/2015/006747, and Weissman and Kariko (2015), each of which is hereby incorporated by reference.

As used herein, “contiguous nucleotides” set forth in a SEQ ID NO refers to nucleotides in a sequence of nucleotides in the order set forth in the SEQ ID NO without any intervening nucleotides.

In embodiments of the present invention, the guide sequence portion may be 25 nucleotides in length and contain 20-22 contiguous nucleotides in the sequence set forth in any one of SEQ ID NOs: 1-103,606. In embodiments of the present invention, the guide sequence portion may be less than 22 nucleotides in length. For example, in embodiments of the present invention the guide sequence portion may be 17, 18, 19, 20, or 21 nucleotides in length. In such embodiments the guide sequence portion may consist of 17, 18, 19, 20, or 21 nucleotides, respectively, in the sequence of 17-22 contiguous nucleotides set forth in any one of SEQ ID NOs: 1-103,606. For example, a guide sequence portion having 17 nucleotides in the sequence of 17 contiguous nucleotides set forth in SEQ ID NO: 103,605 may consist of any one of the following nucleotide sequences (nucleotides excluded from the contiguous sequence are marked in strike-through):

(SEQ ID NO: 103, 607)
AAAAAAAUGUACUUGGUUCC
17 nucleotide guide sequence 1:
(SEQ ID NO: 103, 608) [symbol]
AAAAUGUACUUGGUUCC
17 nucleotide guide sequence 2:
(SEQ ID NO: 103, 609) [symbol]
AAAAAUGUACUUGGUUC [symbol]
17 nucleotide guide sequence 3:
(SEQ ID NO: 103, 610) [symbol]
AAAAAAUGUACUUGGUU [symbol]
17 nucleotide guide sequence 4:
(SEQ ID NO: 103, 611)
AAAAAAAUGUACUUGGU

[symbol]

In embodiments of the present invention, the guide sequence portion may be greater than 20 nucleotides in length. For example, in embodiments of the present invention the guide sequence portion may be 21, 22, 23, 24, 25, 26, 27, 28, 29, or 30 nucleotides in length. In such embodiments the guide sequence portion comprises 17-50 nucleotides containing the sequence of 20, 21 or 22 contiguous nucleotides set forth in any one of SEQ ID NOs: 1-103,606 and additional nucleotides fully complimentary to a nucleotide or sequence of nucleotides adjacent to the 3′ end of the target sequence, 5′ end of the target sequence, or both.

In embodiments of the present invention a CRISPR nuclease and an RNA molecule comprising a guide sequence portion form a CRISPR complex that binds to a target DNA sequence to effect cleavage of the target DNA sequence. CRISPR nucleases, e.g. Cpf1, may form a CRISPR complex comprising a CRISPR nuclease and RNA molecule without a further tracrRNA molecule. Alternatively, CRISPR nucleases, e.g. Cas9, may form a CRISPR complex between the CRISPR nuclease, an RNA molecule, and a tracrRNA molecule. A guide sequence portion, which comprises a nucleotide sequence that is capable of hybridizing to a specific target DNA sequence, and a sequence portion that participates in CRIPSR nuclease binding, e.g. a tracrRNA sequence portion, can be located on the same RNA molecule. Alternatively, a guide sequence portion may be located on one RNA molecule and a sequence portion that participates in CRIPSR nuclease binding, e.g. a tracrRNA portion, may located on a separate RNA molecule. A single RNA molecule comprising a guide sequence portion (e.g. a DNA-targeting RNA sequence) and at least one CRISPR protein-binding RNA sequence portion (e.g. a tracrRNA sequence portion), can form a complex with a CRISPR nuclease and serve as the DNA-targeting molecule. In some embodiments, a first RNA molecule comprising a DNA-targeting RNA portion, which includes a guide sequence portion, and a second RNA molecule comprising a CRISPR protein-binding RNA sequence interact by base pairing to form an RNA complex that targets the CRISPR nuclease to a DNA target site or, alternatively, are fused together to form an RNA molecule that complexes with the CRISPR nuclease and targets the CRISPR nuclease to a DNA target site.

In embodiments of the present invention, a RNA molecule comprising a guide sequence portion may further comprise the sequence of a tracrRNA molecule. Such embodiments may be designed as a synthetic fusion of the guide portion of the RNA molecule and the trans-activating crRNA (tracrRNA). (See Jinek et al., 2012). In such an embodiment, the RNA molecule is a single guide RNA (sgRNA) molecule. Embodiments of the present invention may also form CRISPR complexes utilizing a separate tracrRNA molecule and a separate RNA molecule comprising a guide sequence portion. In such embodiments the tracrRNA molecule may hybridize with the RNA molecule via basepairing and may be advantageous in certain applications of the invention described herein.

The term “tracr mate sequence” refers to a sequence sufficiently complementary to a tracrRNA molecule so as to hybridize to the tracrRNA via basepairing and promote the formation of a CRISPR complex. (See U.S. Pat. No. 8,906,616). In embodiments of the present invention, the RNA molecule may further comprise a portion having a tracr mate sequence.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Eukaryotic” cells include, but are not limited to, fungal cells (such as yeast), plant cells, animal cells, mammalian cells and human cells.

The term “nuclease” as used herein refers to an enzyme capable of cleaving the phosphodiester bonds between the nucleotide subunits of nucleic acid. A nuclease may be isolated or derived from a natural source. The natural source may be any living organism. Alternatively, a nuclease may be a modified or a synthetic protein which retains the phosphodiester bond cleaving activity. Gene modification can be achieved using a nuclease, for example a CRISPR nuclease.

As used herein, the term “HSC” refers to both hematopoietic stem cells and hematopoietic stem progenitor cells. Non-limiting examples of stem cells include bone marrow cells, myeloid progenitor cells, a multipotent progenitor cells, and lineage restricted progenitor cells.

As used herein, “progenitor cell” refers to a lineage cell that is derived from stem cell and retains mitotic capacity and multipotency (e.g., can differentiate or develop into more than one but not all types of mature lineage of cell). As used herein “hematopoiesis” or “hemopoiesis” refers to the formation and development of various types of blood cells (e.g., red blood cells, megakaryocytes, myeloid cells (e.g., monocytes, macrophages and neutrophil), and lymphocytes) and other formed elements in the body (e.g., in the bone marrow).

The term “single nucleotide polymorphism (SNP) position”, as used herein, refers to a position in which a single nucleotide DNA sequence variation occurs between members of a species, or between paired chromosomes in an individual. In the case that a SNP position exists at paired chromosomes in an individual, a SNP on one of the chromosomes is a “heterozygous SNP.” The term SNP position refers to the particular nucleic acid position where a specific variation occurs and encompasses both a sequence including the variation from the most frequently occurring base at the particular nucleic acid position (also referred to as “SNP” or alternative “ALT”) and a sequence including the most frequently occurring base at the particular nucleic acid position (also referred to as reference, or “REF”). Accordingly, the sequence of a SNP position may reflect a SNP (i.e. an alternative sequence variant relative to a consensus reference sequence within a population), or the reference sequence itself.

According to embodiments of the present invention, there is provided a method for modifying in a cell a mutant allele of the Leucine-rich repeat serine/threonine-protein kinase 2 (LRRK2) gene having a mutation associated with monogenic Parkinson's disease, the method comprising
introducing to the cell a composition comprising:
   at least one CRISPR nuclease or a sequence encoding a CRISPR nuclease; and
   a first RNA molecule comprising a guide sequence portion having 17-50 nucleotides or a nucleotide sequence encoding the same,
wherein a complex of the CRISPR nuclease and the first RNA molecule affects a double strand break in the mutant allele of the LRRK2 gene.

In some embodiments, the first RNA molecule targets the CRISPR nuclease to the mutation associated with monogenic Parkinson's disease.

In some embodiments, the mutation associated with monogenic Parkinson's disease is any one of 12:40225041_C_T, 12:40225058_C_A, 12:40225088_G_A, 12:40225159_G_A, 12:40225233_G_C, 12:40225558_C_T, 12:40225618_T_C, 12:40225628_G_A, 12:40225631_T_C, 12:40232303_A_C, 12:40232345_G_C, 12:40232380_A_C, 12:40232385_T_C, 12:40235634_T_C, 12:40235646_C_G, 12:40235660_A_G, 12:40238078_A_G, 12:40240517_C_T, 12:40240531_T_C, 12:40240594_G_C, 12:40240602_T_C, 12:40243551_CAAT_C, 12:40243556_A_T, 12:40249822_T_G, 12:40249843_C_G, 12:40249854_C_T, 12:40249881_T_C, 12:40249908_A_G, 12:40251273_G_A, 12:40251303_G_A, 12:40251361_A_G, 12:40251366_C_A, 12:40251369_G_A, 12:40251495_C_T, 12:40252906_A_G, 12:40252929_G_A, 12:40252939_C_T, 12:40252952_T_G, 12:40252958_A_C, 12:40252968_G_A, 12:40252974_C_T, 12:40252978_C_T, 12:40252984_C_T, 12:40252988_T_C, 12:40253016_G_A, 12:40257311_A_G, 12:40257341_G_T, 12:40259507_A_G, 12:40259525_A_T, 12:40259534_G_T, 12:40259578_G_A, 12:40263806_A_G, 12:40263875_A_G, 12:40263900_G_A, 12:40263907_T_C, 12:40274646_CC_C, 12:40274881_T_C, 12:40274899_A_G, 12:40274917_G_C, 12:40277933_T_C, 12:40277968_A_C, 12:40277971_A_G, 12:40278025_T_A, 12:40278154_A_G, 12:40278167_C_T, 12:40283881_G_A, 12:40283915_T_C, 12:40283947_C_T, 12:40283966_G_A, 12:40283985_C_A, 12:40283990_T_C, 12:40284061_A_G, 12:40284076_C_A, 12:40284139_T_C, 12:40287363_C_A, 12:40287438_A_G, 12:40287461_A_G, 12:40293555_CTC_CCT, 12:40293607_C_T, 12:40293613_G_A, 12:40293624_G_C, 12:40293626_G_A, 12:40293644_A_G, 12:40294854_T_G, 12:40294862_T_C, 12:40294866_G_T, 12:40294878_C_T, 12:40295448_A_G, 12:40295463_A_G, 12:40295466_G_A, 12:40295535_A_T, 12:40295566_A_G, 12:40295574_A_C, 12:40295581_G_T, 12:40298330_A_G, 12:40298333_C_T, 12:40298346_G_A, 12:40298433_C_G, 12:40298479_G_T, 12:40298488_A_G, 12:40299125_A_G, 12:40299190_C_T, 12:40299196_A_C, 12:40299212_G_A, 12:40299255_T_C, 12:40302866_A_G, 12:40303952_C_A, 12:40303972_C_G, 12:40304000_G_A, 12:40304004_A_G, 12:40304035_A_C, 12:40304040_G_C, 12:40304040_G_T, 12:40304078_G_A, 12:40304141_C_T, 12:40305776_C_T, 12:40305781_T_A, 12:40305791_C_G, 12:40305871_G_A, 12:40305942_G_A, 12:40305946_T_A, 12:40308467_G_T, 12:40308476_A_G, 12:40308481_G_A, 12:40308543_GG_G, 12:40308632_C_A, 12:40308672_G_A, 12:40309174_G_A, 12:40309206_C_T, 12:40309225_A_C, 12:40309226_A_G, 12:40309230_A_G, 12:40309236_G_A, 12:40310434_C_A, 12:40310434_C_G, 12:40310434_C_T, 12:40310435_G_A, 12:40310436_C_T, 12:40310437_G_C, 12:40310440_T_G, 12:40310450_C_T, 12:40310461_G_A, 12:40310471_A_G, 12:40310476_GAT_G, 12:40310515_A_G, 12:40310530_C_T, 12:40310546_G_A, 12:40310554_G_A, 12:40310561_G_A, 12:40314043_G_A, 12:40314059_C_T, 12:40314062_A_G, 12:40314101_C_A, 12:40315266_T_A, 12:40319979_A_T, 12:40319998_T_C, 12:40320043_G_A, 12:40320054_G_C, 12:40320090_A_G, 12:40320094_A_G, 12:40320097_T_C, 12:40320119_A_G, 12:40321114_A_G, 12:40321123_T_C, 12:40322037_C_T, 12:40322038_G_A, 12:40322047_G_A, 12:40322047_G_T, 12:40322134_A_G, 12:40322137_A_C, 12:40322364_T_C, 12:40322386_G_T, 12:40322468_C_A, 12:40323244_C_G, 12:40323255_A_G, 12:40323256_T_C, 12:40323260_G_T, 12:40323270_G_T, 12:40323297_T_C, 12:40328416_G_C, 12:40335008_A_T, 12:40335031_G_A, 12:40335036_C_T, 12:40335037_G_A, 12:40335079_G_T, 12:40340349_C_G, 12:40340361_T_C, 12:40340380_T_C, 12:40340400_G_A, 12:40340404_T_C, 12:40340432_A_G, 12:40340436_A_T, 12:40346818_T_C, 12:40346826_ACTACT_A, 12:40346884_A_G, 12:40348402_G_GTGT, 12:40348475_A_G, 12:40348484_C_T, 12:40351537_A_G, 12:40351572_T_A, 12:40351579_C_T, 12:40351585_G_A, 12:40351605_G_C, 12:40351623_G_T, 12:40351667_C_A, 12:40351680_G_C, 12:40351720_G_C, 12:40351723_A_G, 12:40354304_T_C, 12:40354305_G_T, 12:40354410_G_A, 12:40356126_A_T, 12:40356145_C_T, 12:40359345_C_T, 12:40359422_A_G, 12:40363435_C_T, 12:40363440_C_T, 12:40363541_G_A, 12:40364843_G_A, 12:40364845_G_GGT, 12:40364884_G_A, 12:40364927_A_T, 12:40364960_A_G, 12:40365044_C_T, 12:40367012_T_A, 12:40367050_A_G, 12:40367648_AC_A, 12:40367656_G_T, 12:40367754_T_C, 12:40367849_G_A, 12:40367888_A_C, 12:40367988_C_T, 12:40368042_A_G, 12:40368073_A_C, 12:40368170_C_T, 12:40368262_C_T, 12:40368380_T_A, 12:40368486_G_T, 12:40368516_C_A, 12:40368534_T_G, 12:40368673_A_C, 12:40368736_C_A, 12:40368783_G_T, 12:40368800_T_C, 12:40368807_A_G, 12:40368834_A_G, 12:40368860_C_T, 12:40368905_A_T, 12:40368930_C_T, 12:40368947_C_T, 12:40368951_C_T, 12:40369044_T_C, 12:40369153_T_C, and 12:40369167_G_T.

In some embodiments, the guide sequence portion of the first RNA molecule comprises 17-50 contiguous nucleotides containing nucleotides in the sequence set forth in any one of SEQ ID NOs: 1-103,606 that targets a mutation associated with monogenic Parkinson's disease.

In some embodiments, the first RNA molecule targets the CRISPR nuclease to a SNP position of the mutant allele.

In some embodiments, the SNP position is any one of rs1388598, rs28365207, rs7294619, rs10878226, rs1491943, rs1491942, rs1491941, rs11175645, rs4567538, rs12230685, rs11175655, rs11175656, rs1402299410, rs780970090, rs1479481931, rs71078229, rs11175658, rs3057613, rs35642658, rs74324737, rs2708441, rs10631840, rs35991926, rs61931634, rs11175666, rs751672176, rs36024911, rs377568728, rs1279923177, rs2249281, rs10878244, rs138000936, rs10878245, rs10878246, rs10878247, rs954884, rs10878249, rs10784444, rs6581622, rs17519573, rs35987733, rs868476079, rs1399756142, rs1474763022, rs1450276224, rs1191097078, rs1425272990, rs1477924701, rs2404580, rs2404581, rs1388599, rs1388596, rs10878258, rs12371502, rs11175729, rs17443414, rs1486599371, rs7134379, rs7955902, rs1491939, rs1491938, rs7969677, rs2723265, rs7132171, rs10784451, rs10784452, rs1907633, rs1491936, rs1907632, rs2046928, rs2723264, rs17465870, rs10878278, rs11175766, rs11564183, rs11175769, rs17465912, rs17443552, rs17490817, rs11430638, rs4293189, rs10784461, rs7308720, rs10784462, rs10878284, rs11175780, rs11175784, rs11175787, rs144463374, rs73108329, rs12426891, rs4768224, rs11175793, rs10784470, rs12146857, rs11564207, rs7308193, rs10506150, rs76660489, rs10878299, rs373619252, rs10878302, rs11564129, rs7971935, rs11175836, rs4272849, rs10784485, rs10506151, rs7309197, rs7134572, rs11175843, rs147801391, rs376096297, rs7488873, rs11175845, rs7979341, rs1226327150, rs777452337, rs1339001348, rs11175847, rs112859457, rs17466080, rs11175852, rs10784486, rs73108368, rs398019201, rs17519846, rs17466150, rs11175862, rs4310676, rs11564150, rs4318033, rs4473003, rs1429478461, rs886049356, rs11564270, rs17519881, rs17466178, rs17466185, rs11175902, rs76033741, rs56325408, rs7957754, rs11175911, rs12369882, rs11175913, rs4483664, rs66487867, rs1491426750, rs5006481, rs139565575, rs150276517, rs72446556, rs1186032972, rs12820920, rs7966550, rs12314692, rs11175922, rs73102746, rs10784497, rs17443815, rs7957057, rs76607433, rs28365224, rs7962011, rs10878336, rs11175934, rs11175935, rs17491061, rs11175941, rs10878340, rs10784498, rs10878341, rs10784499, rs17484286, rs146071397, rs10878343, rs11564180, rs11175958, rs11175959, rs113772607, rs11564149, rs11175963, rs7305344, rs7133914, rs11175964, rs397805328, rs11175966, rs10784501, rs41286474, rs869212703, rs2069228, rs10784503, rs386376250, rs11564128, rs2896975, rs7302503, rs7137160, rs369794148, rs34709132, rs10467144, rs11175985, rs10784504, rs57421387, rs7973058, rs1427267, rs1427266, rs721713, rs721712, rs721711, rs721709, rs4768227, rs398098159, rs4767969, rs10878356, rs10784506, rs10878358, rs7132073, rs1427265, rs17466339, rs2114569, rs2114568, rs2162469, rs10784509, rs1896254, rs5797667, rs373676201, rs1896253, rs1896252, rs1427263, rs11176013, rs11564148, rs11564205, rs10748030, rs10748031, rs10748032, rs11564177, rs10748033, rs10735931, rs10748034, rs10878368, rs7307276, rs4768229, rs4768230, rs397968969, rs11564204, rs4423250, rs7137665, rs10878371, rs11176022, rs60750674, rs10878372, rs398055492, rs761931834, rs1415940047, rs17466430, rs11176030, rs3747891, rs7298930, rs17444089, rs10878377, rs12316801, rs17444096, rs11564175, rs2404832, rs17466486, rs11564267, rs17520244, rs10506152, rs11176052, rs11176053, rs17444124, rs11564203, r57307562, rs17520251, rs11461395, rs10506153, rs73275761, rs963243, rs17444145, rs10878386, rs17466521, rs12367542, rs17520278, rs35031086, rs11834950, rs11829088, rs4465428, rs61007767, rs60776971, rs2404834, rs7302841, rs2404835, rs11356084, rs715403, rs715402, rs6581667, rs6581668, rs200521371, rs4767970, rs4767971, rs4767972, rs1427271, rs1427272, rs1427273, rs10735934, rs10732751, rs12306060, rs1365763, rs7963697, rs11316380, rs7963086, rs17491536, rs7956787, rs7956898, rs10506155, rs7954061, rs7957151, rs919174, rs3065921, rs919175, rs1035812, rs1365764, rs397724721, rs17461964, rs10784518, rs4768231, rs4768232, rs3943893, rs2162471, rs1035813, rs10459264, rs10459265, rs11564147, rs10715759, rs7296657, rs7312497, rs10715758, rs10784522, rs796827140, rs74467833, rs7131694, rs12311273, rs58911468, rs35847030, rs1365765, rs66513412, rs2896977, rs199606989, rs1365766, rs10878405, rs11176143, rs7294958, rs61915610, rs7294952, rs59096461, rs59980086, rs4768233, rs4768234, rs4768235, rs7314455, rs7313525, rs7313895, rs7314863, rs7303525, rs7132187, rs7307310, rs11176153, rs11176154, rs7311247, rs7137437, rs75439407, rs73107108, rs1307806388, rs10878409, rs10878410, rs138382676, rs11176161, rs10878412, rs10878413, rs183544696, rs189538849, rs11610569, rs142726158, rs1194726090, rs576444406, rs185723137, rs1179215763, rs71449759, rs61915613, rs140722234, rs191099092, rs113693842, rs112767992, rs34531686, rs12368227, rs12368229, rs3930031, rs7308626, rs890575, rs890575, rs10748040, rs61230125, rs11176165, rs7976715, rs7976724, rs7963987, rs2162472, rs767501990, rs552326490, rs1365381164, rs760911090, rs754028820, rs11564173, rs7306545, rs7306684, rs112259589, rs73277531, rs77689380, rs73277533, rs17462055, rs758880647, rs10784528, rs796949072, rs10784532, rs145580704, rs386376254, rs11836288, rs11564146, rs10784536, rs11277474, rs7971919, rs7962116, rs67512688, rs2896978, rs2404836, rs11176195, rs4767973, rs17444202, rs4768236, rs4768237, rs58724345, rs397897325, rs33962975, rs7134408, rs11835105, rs137858615, rs10878434, rs35403247, rs3761863, rs17444223, rs12426362, rs17466605, rs3789328, rs12426498, rs4768238, rs66737902, rs10878441, rs3886747, rs1365770, rs367772598, rs386376255, rs10784548, rs1465527, rs1465528, rs17444285, rs7968048, rs11609433, rs776238897, rs149833872, rs1299545494, rs4374003, and rs7962370.

In some embodiments, the guide sequence portion of the first RNA molecule comprises 17-50 contiguous nucleotides containing nucleotides in the sequence set forth in any one of SEQ ID NOs: 1-103,606 that targets a SNP position of the mutant allele.

In some embodiments, the SNP position is in an exon or intron of the LRRK2 mutant allele, or within 3,000 basepairs upstream of the transcription start site or within 3,000 basepairs downstream of the 3′ UTR of the LRRK2 mutant allele.

In some embodiments, the SNP position contains a heterozygous SNP.

In some embodiments, the method further comprises introducing to the cell a second RNA molecule comprising a guide sequence portion having 17-50 nucleotides or a nucleotide sequence encoding the same, wherein a complex of the second RNA molecule and a CRISPR nuclease affects a second double strand break in the LRRK2 gene.

In some embodiments, the guide sequence portion of the second RNA molecule comprises 17-50 contiguous nucleotides containing nucleotides in the sequence set forth in any one of SEQ ID NOs: 1-103,606 other than the sequence of the first RNA molecule.

In some embodiments, the second RNA molecule comprises a non-discriminatory guide portion that targets both functional and mutated LRRK2 alleles.

In some embodiments, the second RNA molecule comprises a non-discriminatory guide portion that targets any one of Intron 1 of LRRK2, Intron 49 of LRRK2, Intron 2 of LRRK2, Intron 3 of LRRK2, a 3′ untranslated region (3′ UTR) of LRRK2, and an intergenic region downstream of LRRK2.

In some embodiments, the second RNA molecule comprises a non-discriminatory guide portion that targets a sequence that is located within a genomic range selected from any one of 12:40224997-40225496, 12:40225283-40225554, 12:40366506-40367005, 12:40367078-40367643, 12:40225641-40226140, and 12:40235126-40235625.

In some embodiments, the second RNA molecule comprises a non-discriminatory guide portion that targets a sequence that is located up to 500 base pairs from an exon that is excised by the first and second RNA molecules.

In some embodiments, an exon or a portion thereof is excised from the mutant allele of the LRRK2 gene.

In some embodiments, the first RNA molecule targets a SNP position in the 3′ UTR of the mutated allele, and the second RNA molecule comprises a non-discriminatory guide portion that targets downstream of a polyadenylation signal sequence that is common to both a functional allele and the mutant allele of the LRRK2 gene.

In some embodiments, the first RNA molecule targets a SNP position downstream of a polyadenylation signal of the mutated allele, and the second RNA molecule comprises a non-discriminatory guide portion that targets a sequence upstream of a polyadenylation signal that is common to both a functional allele and the mutant allele of the LRRK2 gene.

In some embodiments, the polyadenylation signal is excised from the mutant allele of the LRRK2 gene.

In some embodiments, the guide sequence portion of the first RNA molecule or second RNA molecule comprises 1, 2, 3, 4, or 5 nucleotide mismatches relative to a fully complementary target sequence of the guide sequence portion.

In some embodiments, the guide sequence portion comprises 17-50 contiguous nucleotides containing nucleotides in the sequence set forth in any one of SEQ ID NOs: 1-103,606 modified to contain 1, 2, 3, 4, or 5 nucleotide mismatches relative to a fully complementary target sequence of the guide sequence portion.

In some embodiments, the guide sequence portion provides higher targeting specificity to the complex of the CRISPR nuclease and the first RNA molecule relative to a guide sequence portion that has higher complementarity to the mutant allele of the LRRK2 gene

According to embodiments of the present invention, there is provided a modified cell obtained by the method of any one of the embodiments presented herein. In some embodiments the modified cell is a stem cell, a neuron, or a dopaminergic cell.

According to embodiments of the present invention, there is provided a first RNA molecule comprising a guide sequence portion having 17-50 contiguous nucleotides containing nucleotides in the sequence set forth in any one of SEQ ID NOs: 1-103,606, or any one of SEQ ID NOs: 1-103,606 modified to contain 1, 2, 3, 4, or 5 nucleotide mismatches relative to a fully complementary target sequence of the guide sequence portion.

According to embodiments of the present invention, there is provided a composition comprising the first RNA molecule and at least one CRISPR nuclease.

In some embodiments, the composition further comprises a second RNA molecule comprising a guide sequence portion having 17-50 contiguous nucleotides, wherein the second RNA molecule targets a LRRK2 allele, and wherein the guide sequence portion of the second RNA molecule is a different sequence from the sequence of the guide sequence portion of the first RNA molecule.

In some embodiments, the guide sequence portion of the second RNA molecule comprises 17-50 contiguous nucleotides containing nucleotides in the sequence set forth in any one of SEQ ID NOs: 1-103,606 other than the sequence of the first RNA molecule, or any one of SEQ ID NOs: 1-103,606 modified to contain 1, 2, 3, 4, or 5 nucleotide mismatches relative to a fully complementary target sequence of the guide sequence portion.

According to embodiments of the present invention, there is provided a method for inactivating a mutant LRRK2 allele in a cell, the method comprising delivering to the cell the composition of any one of the embodiments presented herein.

According to embodiments of the present invention, there is provided a method for treating monogenic Parkinson's disease, the method comprising delivering to a cell of a subject having monogenic Parkinson's disease the composition of any one of the embodiments presented herein.

According to embodiments of the present invention, there is provided use of any one of the compositions presented herein for inactivating a mutant LRRK2 allele in a cell, comprising delivering to the cell the composition of any one of the embodiments presented herein.

According to embodiments of the present invention, there is provided a medicament comprising the composition of any one of the embodiments presented herein for use in inactivating a mutant LRRK2 allele in a cell, wherein the medicament is administered by delivering to the cell the composition of any one of the embodiments presented herein.

According to embodiments of the present invention, there is provided use of the composition of any one of the embodiments presented herein for treating ameliorating or preventing monogenic Parkinson's disease, comprising delivering to a cell of a subject having or at risk of having monogenic Parkinson's disease the composition of any one of the embodiments presented herein.

According to embodiments of the present invention, there is provided a medicament comprising the composition of any one of the embodiments presented herein for use in treating ameliorating or preventing monogenic Parkinson's disease, wherein the medicament is administered by delivering to a cell of a subject having or at risk of having monogenic Parkinson's disease the composition of any one of the embodiments presented herein.

According to embodiments of the present invention, there is provided a kit for inactivating a mutant LRRK2 allele in a cell, comprising an RNA molecule of any one of the embodiments presented herein, a CRISPR nuclease, and/or a tracrRNA molecule; and instructions for delivering the RNA molecule; CRISPR nuclease, and/or the tracrRNA to the cell.

According to embodiments of the present invention, there is provided a kit for treating monogenic Parkinson's disease in a subject, comprising an RNA molecule of any one of the embodiments presented herein, a CRISPR nuclease, and/or a tracrRNA molecule; and instructions for delivering the RNA molecule; CRISPR nuclease, and/or the tracrRNA to a cell of a subject having or at risk of having monogenic Parkinson's disease.

According to embodiments of the present invention, there is provided a gene editing composition comprising an RNA molecule comprising a guide sequence portion having 17-50 contiguous nucleotides containing nucleotides in the sequence set forth in any one of SEQ ID NOs: 1-103,606. In some embodiments, the RNA molecule further comprises a portion having a sequence which binds to a CRISPR nuclease. In some embodiments, the sequence which binds to a CRISPR nuclease is a tracrRNA sequence.

In some embodiments, the RNA molecule further comprises a portion having a tracr mate sequence.

In some embodiments, the RNA molecule may further comprise one or more linker portions.

According to embodiments of the present invention, an RNA molecule may be up to 1000, 900, 800, 700, 600, 500, 450, 400, 350, 300, 290, 280, 270, 260, 250, 240, 230, 220, 210, 200, 190, 180, 170, 160, 150, 140, 130, 120, 110, or 100 nucleotides in length. Each possibility represents a separate embodiment. In embodiments of the present invention, the RNA molecule may be 17 up to 300 nucleotides in length, 100 up to 300 nucleotides in length, 150 up to 300 nucleotides in length, 100 up to 500 nucleotides in length, 100 up to 400 nucleotides in length, 200 up to 300 nucleotides in length, 100 to 200 nucleotides in length, or 150 up to 250 nucleotides in length. Each possibility represents a separate embodiment.

According to some embodiments of the present invention, the composition further comprises a tracrRNA molecule.

The present disclosure provides a method for utilizing at least one naturally occurring nucleotide difference or polymorphism (e.g., single nucleotide polymorphism (SNP)) for distinguishing/discriminating between two alleles of a gene, one allele bearing a mutation such that it encodes a mutated protein causing a disease phenotype (“mutated allele”) and a particular sequence in a SNP position (SNP/REF), and the other allele encoding for a functional protein (“functional allele”). The method further comprises the step of knocking out expression of the mutated protein and allowing expression of the functional protein. In some embodiments, the method is for treating, ameliorating, or preventing a dominant negative genetic disorder.

According to some embodiments of the present invention, there is provided a method for inactivating a mutant LRRK2 allele in a cell, the method comprising delivering to the cell a composition comprising an RNA molecule comprising a guide sequence portion having 17-50 contiguous nucleotides containing nucleotides in the sequence set forth in any one of SEQ ID NOs: 1-103,606 and a CRISPR nuclease.

According to some embodiments of the present invention, there is provided a method for treating monogenic Parkinson's disease, the method comprising delivering to a cell of a subject having monogenic Parkinson's disease a composition comprising an RNA molecule comprising a guide sequence portion having 17-50 contiguous nucleotides containing nucleotides in the sequence set forth in any one of SEQ ID NOs: 1-103,606 and a CRISPR nuclease.

According to embodiments of the present invention, the composition comprises a second RNA molecule comprising a guide sequence portion having 17-50 contiguous nucleotides containing nucleotides in the sequence set forth in any one of SEQ ID NOs: 1-103,606. In some embodiments, the 17-50 nucleotides of the guide sequence portion of the second RNA molecule are in a different sequence from the sequence of the guide sequence portion of the first RNA molecule.

According to embodiments of the present invention, at least one CRISPR nuclease and the RNA molecule or RNA molecules are delivered to the subject and/or cells substantially at the same time or at different times.

In some embodiments, a tracrRNA molecule is delivered to the subject and/or cells substantially at the same time or at different times as the CRISPR nuclease and RNA molecule or RNA molecules.

According to embodiments of the present invention, the first RNA molecule targets a SNP or disease-causing mutation in the exon or promoter of a mutated allele, and the second RNA molecule targets a SNP in an exon of the mutated allele, a SNP in an intron, or a sequence present in both the mutated or functional allele.

According to embodiments of the present invention, the first RNA molecule or the first and the second RNA molecules target a SNP in the promoter region, the start codon, or an untranslated region (UTR) of a mutated allele.

According to embodiments of the present invention, the first RNA molecule or the first and the second RNA molecules targets at least a portion of the promoter and/or the start codon and/or a portion of a UTR of a mutated allele.

According to embodiments of the present invention, the first RNA molecule targets a portion of the promoter, a first SNP in the promoter, or a SNP upstream to the promoter of a mutated allele and the second RNA molecule is targets a second SNP, which is downstream of the first SNP, and is in the promoter, in a UTR, or in an intron or in an exon of a mutated allele.

According to embodiments of the present invention, the first RNA molecule targets a SNP in the promoter, upstream of the promoter, or a UTR of a mutated allele and the second RNA molecule is designed to target a sequence which is present in an intron of both the mutated allele and the functional allele.

According to embodiments of the present invention, the first RNA molecule targets a SNP in an intron of a mutated allele, and wherein the second RNA molecule targets a SNP in an intron of the mutated allele, or a sequence in an intron present in both the mutated and functional allele.

According to embodiments of the present invention, the first RNA molecule targets a sequence upstream of the promotor which is present in both a mutated and functional allele and the second RNA molecule targets a SNP or disease-causing mutation in any location of the gene.

According to embodiments of the present invention, there is provided a method comprising removing an exon containing a disease-causing mutation from a mutated allele, wherein the first RNA molecule or the first and the second RNA molecules target regions flanking an entire exon or a portion of the exon.

According to embodiments of the present invention, there is provided a method comprising removing an exon or a portion thereof from a mutant LRRK2 allele, the entire open reading frame of a mutant LRRK2 allele, or removing the entire mutant LRRK2 allele.

According to embodiments of the present invention, the first RNA molecule targets a SNP or disease-causing mutation in an exon or promoter of a mutated allele, and wherein the second RNA molecule targets a SNP in the same exon of the mutated allele, a SNP in an intron, or a sequence in an intron present in both the mutated and functional allele.

According to embodiments of the present invention, the first RNA molecule or the first and the second RNA molecules target an alternative splicing signal sequence between an exon and an intron of a mutant LRRK2 allele.

According to embodiments of the present invention, the second RNA molecule is non-discriminatory targets a sequence present in both a mutated allele and a functional allele.

The compositions and methods of the present disclosure may be utilized for treating, preventing, ameliorating, or slowing progression of an autosomal dominant genetic disorder, such as monogenic Parkinson's disease.

In some embodiments, a mutated allele is deactivated by delivering to a cell an RNA molecule which targets a SNP in the promoter region, the start codon, or an untranslated region (UTR) of the mutated allele.

In some embodiments, a mutated allele is inactivated by removing at least a portion of the promoter, and/or removing the start codon, and/or a portion of the UTR, and/or a polyadenylation signal. In such embodiments one RNA molecule may be designed for targeting a first SNP in the promoter or upstream to the promoter and another RNA molecule is designed to target a second SNP, which is downstream of the first SNP, and is in the promoter, in the UTR, in an intron, or in an exon. Alternatively, one RNA molecule may be designed for targeting a SNP in the promoter, upstream of the promoter, or the UTR, and another RNA molecule is designed to target a sequence which is present in an intron of both the mutated allele and the functional allele. Alternatively, one RNA molecule may be designed for targeting a sequence upstream of the promotor which is present in both the mutated and functional allele and the other guide is designed to target a SNP or disease-causing mutation in any location of the gene e.g., in an exon, intron, UTR, or downstream of the promoter.

In some embodiments, the method of deactivating a mutated allele comprises an exon skipping step comprising removing an exon containing a disease-causing mutation from the mutated allele. Removing an exon containing a disease-causing mutation in the mutated allele requires two RNA molecules which target regions flanking the entire exon or a portion of the exon. Removal of an exon containing the disease-causing mutation may be designed to eliminate the disease-causing action of the protein while allowing for expression of the remaining protein product which retains some or all of the wild-type activity. The entire open reading frame or the entire gene can be excised using two RNA molecules flanking the region desired to be excised.

In some embodiments, the method of deactivating a mutated allele comprises delivering two RNA molecules to a cell, wherein one RNA molecule targets a SNP or disease-causing mutation in an exon or promoter of the mutated allele, and wherein the other RNA molecule targets a SNP in the same of the mutated allele, a SNP in an intron, or a sequence in an intron present in both the mutated or functional allele.

Any one of, or combination of, the above-mentioned strategies for deactivating a mutant allele may be used in the context of the invention.

In embodiments of the present invention, an RNA molecule is used to direct a CRISPR nuclease to an exon or a splice site of a mutated allele in order to create a double-stranded break (DSB), leading to insertion or deletion of nucleotides by inducing an error-prone non-homologous end-joining (NHEJ) mechanism and formation of a frameshift mutation in the mutated allele. The frameshift mutation may result in, for example, inactivation or knockout of the mutated allele by generation of an early stop codon in the mutated allele and to generation of a truncated protein or to nonsense-mediated mRNA decay of the transcript of the mutant allele. In further embodiments, one RNA molecule is used to direct a CRISPR nuclease to a promotor of a mutated allele.

In some embodiments, the method of deactivating a mutated allele further comprises enhancing activity of the functional protein such as by providing a protein/peptide, a nucleic acid encoding a protein/peptide, or a small molecule such as a chemical compound, capable of activating/enhancing activity of the functional protein.

According to some embodiments, the present disclosure provides an RNA sequence (also referred to as an ‘RNA molecule’) which binds to or associates with and/or directs an RNA-guided DNA nuclease e.g., a CRISPR nuclease, to a target sequence comprising at least one nucleotide which differs between a mutated allele and a functional allele (e.g., SNP) of a gene of interest (i.e., a sequence of the mutated allele which is not present in the functional allele).

In some embodiments, the method comprises contacting a mutated allele of a gene of interest with an allele-specific RNA molecule and a CRISPR nuclease e.g., a Cas9 protein, wherein the allele-specific RNA molecule and the CRISPR nuclease associate with a nucleotide sequence of the mutated allele of the gene of interest which differs by at least one nucleotide from a nucleotide sequence of a functional allele of the gene of interest, thereby modifying or knocking-out the mutated allele.

In some embodiments, the allele-specific RNA molecule and a CRISPR nuclease is introduced to a cell encoding the gene of interest. In some embodiments, the cell encoding the gene of interest is in a mammalian subject. In some embodiments, the cell encoding the gene of interest is in a plant.

In some embodiments, the mutated allele is an allele of LRRK2 gene. In some embodiments, the RNA molecule targets a SNP which co-exists with or is genetically linked to the mutated sequence associated with monogenic Parkinson's disease genetic disorder. In some embodiments, the RNA molecule targets a SNP which is highly prevalent in the population and exists in the mutated allele having the mutated sequence associated with monogenic Parkinson's disease genetic disorder and not in the functional allele of an individual subject to be treated. In some embodiments, a disease-causing mutation within a mutated LRRK2 allele is targeted.

In some embodiments, the SNP is within an exon of the gene of interest. In such embodiments, a guide sequence portion of an RNA molecule is designed to associate with a sequence of an exon of the gene of interest.

In some embodiments, SNP is within an intron or the exon of the gene of interest. In some embodiments, the SNP is in close proximity to the splice site between an intron and an exon. In some embodiments, the close proximity to a splice site is 1, 2, 3, 4, 5, 6, 7, 8, 9, 10, 11, 12, 13, 14, 15, 16, 17, 18, 19, or 20 nucleotides upstream or downstream to the splice site. Each possibility represents a separate embodiment of the present invention. In such embodiments, a guide sequence portion of an RNA molecule may be designed to associate with a sequence of the gene of interest which comprises the splice site.

In some embodiments, the method is utilized for treating a subject having a disease phenotype resulting from the heterozygote LRRK2 gene. In such embodiments, the method results in improvement, amelioration or prevention of the disease phenotype.

Embodiments of compositions described herein include at least one CRISPR nuclease, RNA molecule(s), and a tracrRNA molecule, being effective in a subject or cells at the same time. The at least one CRISPR nuclease, RNA molecule(s), and tracrRNA may be delivered substantially at the same time or can be delivered at different times but have effect at the same time. For example, this includes delivering the CRISPR nuclease to the subject or cells before the RNA molecule and/or tracrRNA is substantially extant in the subject or cells.

In some embodiments, the cell is a stem cell. In some embodiments, the cell is a neuron. In some embodiments, the cell is a dopaminergic cell.

### Dominant Genetic Disorders

One of skill in the art will appreciate that all subjects with any type of heterozygote genetic disorder (e.g., dominant genetic disorder) may be subjected to the methods described herein. In one embodiment, the present invention may be used to target a gene involved in, associated with, or causative of dominant genetic disorders such as, for example, monogenic Parkinson's disease. In some embodiments, the dominant genetic disorder is monogenic Parkinson's disease. In some embodiments, the target gene is the LRRK2 gene. Non-limiting examples of mutations characterized as gain of function mutations associated with monogenic Parkinson's disease phenotype include: 12:40225041_C_T, 12:40225058_C_A, 12:40225088_G_A, 12:40225159_G_A, 12:40225233_G_C, 12:40225558_C_T, 12:40225618_T_C, 12:40225628_G_A, 12:40225631_T_C, 12:40232303_A_C, 12:40232345_G_C, 12:40232380_A_C, 12:40232385_T_C, 12:40235634_T_C, 12:40235646_C_G, 12:40235660_A_G, 12:40238078_A_G, 12:40240517_C_T, 12:40240531_T_C, 12:40240594_G_C, 12:40240602_T_C, 12:40243551_CAAT_C, 12:40243556_A_T, 12:40249822_T_G, 12:40249843_C_G, 12:40249854_C_T, 12:40249881_T_C, 12:40249908_A_G, 12:40251273_G_A, 12:40251303_G_A, 12:40251361_A_G, 12:40251366_C_A, 12:40251369_G_A, 12:40251495_C_T, 12:40252906_A_G, 12:40252929_G_A, 12:40252939_C_T, 12:40252952_T_G, 12:40252958_A_C, 12:40252968_G_A, 12:40252974_C_T, 12:40252978_C_T, 12:40252984_C_T, 12:40252988_T_C, 12:40253016_G_A, 12:40257311_A_G, 12:40257341_G_T, 12:40259507_A_G, 12:40259525_A_T, 12:40259534_G_T, 12:40259578_G_A, 12:40263806_A_G, 12:40263875_A_G, 12:40263900_G_A, 12:40263907_T_C, 12:40274646_CC_C, 12:40274881_T_C, 12:40274899_A_G, 12:40274917_G_C, 12:40277933_T_C, 12:40277968_A_C, 12:40277971_A_G, 12:40278025_T_A, 12:40278154_A_G, 12:40278167_C_T, 12:40283881_G_A, 12:40283915_T_C, 12:40283947_C_T, 12:40283966_G_A, 12:40283985_C_A, 12:40283990_T_C, 12:40284061_A_G, 12:40284076_C_A, 12:40284139_T_C, 12:40287363_C_A, 12:40287438_A_G, 12:40287461_A_G, 12:40293555_CTC_CCT, 12:40293607_C_T, 12:40293613_G_A, 12:40293624_G_C, 12:40293626_G_A, 12:40293644_A_G, 12:40294854_T_G, 12:40294862_T_C, 12:40294866_G_T, 12:40294878_C_T, 12:40295448_A_G, 12:40295463_A_G, 12:40295466_G_A, 12:40295535_A_T, 12:40295566_A_G, 12:40295574_A_C, 12:40295581_G_T, 12:40298330_A_G, 12:40298333_C_T, 12:40298346_G_A, 12:40298433_C_G, 12:40298479_G_T, 12:40298488_A_G, 12:40299125_A_G, 12:40299190_C_T, 12:40299196_A_C, 12:40299212_G_A, 12:40299255_T_C, 12:40302866_A_G, 12:40303952_C_A, 12:40303972_C_G, 12:40304000_G_A, 12:40304004_A_G, 12:40304035_A_C, 12:40304040_G_C, 12:40304040_G_T, 12:40304078_G_A, 12:40304141_C_T, 12:40305776_C_T, 12:40305781_T_A, 12:40305791_C_G, 12:40305871_G_A, 12:40305942_G_A, 12:40305946_T_A, 12:40308467_G_T, 12:40308476_A_G, 12:40308481_G_A, 12:40308543_GG_G, 12:40308632_C_A, 12:40308672_G_A, 12:40309174_G_A, 12:40309206_C_T, 12:40309225_A_C, 12:40309226_A_G, 12:40309230_A_G, 12:40309236_G_A, 12:40310434_C_A, 12:40310434_C_G, 12:40310434_C_T, 12:40310435_G_A, 12:40310436_C_T, 12:40310437_G_C, 12:40310440_T_G, 12:40310450_C_T, 12:40310461_G_A, 12:40310471_A_G, 12:40310476_GAT_G, 12:40310515_A_G, 12:40310530_C_T, 12:40310546_G_A, 12:40310554_G_A, 12:40310561_G_A, 12:40314043_G_A, 12:40314059_C_T, 12:40314062_A_G, 12:40314101_C_A, 12:40315266_T_A, 12:40319979_A_T, 12:40319998_T_C, 12:40320043_G_A, 12:40320054_G_C, 12:40320090_A_G, 12:40320094_A_G, 12:40320097_T_C, 12:40320119_A_G, 12:40321114_A_G, 12:40321123_T_C, 12:40322037_C_T, 12:40322038_G_A, 12:40322047_G_A, 12:40322047_G_T, 12:40322134_A_G, 12:40322137_A_C, 12:40322364_T_C, 12:40322386_G_T, 12:40322468_C_A, 12:40323244_C_G, 12:40323255_A_G, 12:40323256_T_C, 12:40323260_G_T, 12:40323270_G_T, 12:40323297_T_C, 12:40328416_G_C, 12:40335008_A_T, 12:40335031_G_A, 12:40335036_C_T, 12:40335037_G_A, 12:40335079_G_T, 12:40340349_C_G, 12:40340361_T_C, 12:40340380_T_C, 12:40340400_G_A, 12:40340404_T_C, 12:40340432_A_G, 12:40340436_A_T, 12:40346818_T_C, 12:40346826_ACTACT_A, 12:40346884_A_G, 12:40348402_G_GTGT, 12:40348475_A_G, 12:40348484_C_T, 12:40351537_A_G, 12:40351572_T_A, 12:40351579_C_T, 12:40351585_G_A, 12:40351605_G_C, 12:40351623_G_T, 12:40351667_C_A, 12:40351680_G_C, 12:40351720_G_C, 12:40351723_A_G, 12:40354304_T_C, 12:40354305_G_T, 12:40354410_G_A, 12:40356126_A_T, 12:40356145_C_T, 12:40359345_C_T, 12:40359422_A_G, 12:40363435_C_T, 12:40363440_C_T, 12:40363541_G_A, 12:40364843_G_A, 12:40364845_G_GGT, 12:40364884_G_A, 12:40364927_A_T, 12:40364960_A_G, 12:40365044_C_T, 12:40367012_T_A, 12:40367050_A_G, 12:40367648_AC_A, 12:40367656_G_T, 12:40367754_T_C, 12:40367849_G_A, 12:40367888_A_C, 12:40367988_C_T, 12:40368042_A_G, 12:40368073_A_C, 12:40368170_C_T, 12:40368262_C_T, 12:40368380_T_A, 12:40368486_G_T, 12:40368516_C_A, 12:40368534_T_G, 12:40368673_A_C, 12:40368736_C_A, 12:40368783_G_T, 12:40368800_T_C, 12:40368807_A_G, 12:40368834_A_G, 12:40368860_C_T, 12:40368905_A_T, 12:40368930_C_T, 12:40368947_C_T, 12:40368951_C_T, 12:40369044_T_C, 12:40369153_T_C, and 12:40369167_G_T.

LRRK2 editing strategies include, but are not limited to, (1) knockout, for example, by targeting a LRRK2 mutation or SNP position with one guide RNA molecule to induce a frameshift that may lead to truncation or nonsense-mediated decay; and (2) allele-specific excision using two guide RNA molecules, for example, excision of at least one exon or a portion thereof, knockout of a large portion of the allele or the entire allele, or excision of the polyadenylation signal or the promoter region.

Knockout may be achieved by several approaches. For example, knockout may be achieved by targeting a SNP within a coding exon of a mutant LRRK2 allele using only one guide RNA molecule (e.g. a single guide RNA molecule or “sgRNA”). Alternatively, excision may be achieved by targeting the mutant LRRK2 allele with two different RNA molecules, with at least one RNA molecule being allele-specific.

In another editing strategy, expression of a mutated LRRK2 allele may be inhibited. An example of this strategy includes excising the polyadenylation signal in the 3′UTR region, which leads to an unstable transcript.

According to embodiments of the present invention, there is provided an RNA molecule comprising a guide sequence portion (e.g. a targeting sequence) comprising a nucleotide sequence that is fully or partially complementary to a target sequence in a SNP position (REF/SNP sequence) located in or near a mutated allele of the LRRK2 gene. In some embodiments, the guide sequence portion of the RNA molecule consists of 16, 17, 18, 19, 20, 21, 22, 23, 24, 25, 26, or more than 26 nucleotides. In some embodiments the guide sequence portion is configured to target a CRISPR nuclease to a target sequence and provide a cleavage event, by a CRISPR nuclease complexed therewith, selected from a double-strand break and a single-strand break within 500, 400, 300, 200, 100, 50, 25, or 10 nucleotides of a LRRK2 target site. In some embodiments, the cleavage event enables non-sense mediated decay of the LRRK2 gene. In some embodiments, the RNA molecule is a guide RNA molecule such as a crRNA molecule or a single guide RNA molecule.

In some embodiments, the target sequence of a mutated allele of LRRK2 gene is altered (e.g., by introduction of an NHEJ-mediated indel (e.g., insertion or deletion), and results in reduction or elimination of expression of the gene product encoded by the mutant allele of LRRK2 gene. In some embodiments, the reduction or elimination of expression is due to non-sense mediated mRNA decay such as due to immature stop codon. In some embodiments, the reduction or elimination of expression is due to expression of a truncated form of the LRRK2 gene product.

### CRISPR Nucleases and PAM Recognition

In some embodiments, the sequence specific nuclease is selected from CRISPR nucleases, or a functional variant thereof. In some embodiments, the sequence specific nuclease is an RNA guided DNA nuclease. In such embodiments, the RNA sequence which guides the RNA guided DNA nuclease (e.g., Cpf1) binds to and/or directs the RNA guided DNA nuclease to the sequence comprising at least one nucleotide which differs between a mutated allele and its counterpart functional allele (e.g., SNP). In some embodiments, the CRISPR complex does not further comprise a tracrRNA. In a non-limiting example, in which the RNA guided DNA nuclease is a CRISPR protein, the at least one nucleotide which differs between the dominant mutated allele and the functional allele may be within the PAM site and/or proximal to the PAM site within the region that the RNA molecule is designed to hybridize to. A skilled artisan will appreciate that RNA molecules can be engineered to bind to a target of choice in a genome by commonly known methods in the art.

The term “PAM” as used herein refers to a nucleotide sequence of a target DNA located in proximity to the targeted DNA sequence and recognized by the CRISPR nuclease complex. The PAM sequence may differ depending on the nuclease identity. In addition, there are CRISPR nucleases that can target almost all PAMs. In some embodiments of the present invention, a CRISPR system utilizes one or more RNA molecules having a guide sequence portion to direct a CRISPR nuclease to a target DNA site via Watson-Crick base-pairing between the guide sequence portion and the protospacer on the target DNA site, which is next to the protospacer adjacent motif (PAM), which is an additional requirement for target recognition. The CRISPR nuclease then mediates cleavage of the target DNA site to create a double-stranded break within the protospacer. In a non-limiting example, a type II CRISPR system utilizes a mature crRNA:tracrRNA complex that directs the CRISPR nuclease, e.g. Cas9 to the target DNA the target DNA via Watson-Crick base-pairing between the guide sequence portion of the crRNA and the protospacer on the target DNA next to the protospacer adjacent motif (PAM). A skilled artisan will appreciate that each of the engineered RNA molecule of the present invention is further designed such as to associate with a target genomic DNA sequence of interest next to a protospacer adjacent motif (PAM), e.g., a PAM matching the sequence relevant for the type of CRISPR nuclease utilized, such as for a non-limiting example, NGG or NAG, wherein “N” is any nucleobase, for Streptococcus pyogenes Cas9 WT (SpCAS9); NNGRRT for Staphylococcus aureus (SaCas9); NNNVRYM for Jejuni Cas9 WT; NGAN or NGNG for SpCas9-VQR variant; NGCG for SpCas9-VRER variant; NGAG for SpCas9-EQR variant; NRRH for SpCas9-NRRH variant, wherein N is any nucleobase, R is A or G and H is A, C, or T; NRTH for SpCas9-NRTH variant, wherein N is any nucleobase, R is A or G and H is A, C, or T; NRCH for SpCas9-NRCH variant, wherein N is any nucleobase, R is A or G and H is A, C, or T; NG for SpG variant of SpCas9 wherein N is any nucleobase; NG or NA for SpCas9-NG variant of SpCas9 wherein N is any nucleobase; NR or NRN or NYN for SpRY variant of SpCas9, wherein N is any nucleobase, R is A or G and Y is C or T; NNG for Streptococcus canis Cas9 variant (ScCas9), wherein N is any nucleobase; NNNRRT for SaKKH-Cas9 variant of Staphylococcus aureus (SaCas9), wherein N is any nucleobase, and R is A or G; NNNNGATT for Neisseria meningitidis (NmCas9), wherein N is any nucleobase; TTN for Alicyclobacillus acidiphilus Cas12b (AacCas12b), wherein N is any nucleobase; or TTTV for Cpf1, wherein V is A, C or G. RNA molecules of the present invention are each designed to form complexes in conjunction with one or more different CRISPR nucleases and designed to target polynucleotide sequences of interest utilizing one or more different PAM sequences respective to the CRISPR nuclease utilized.

In some embodiments, an RNA-guided DNA nuclease e.g., a CRISPR nuclease, may be used to cause a DNA break, either double or single-stranded in nature, at a desired location in the genome of a cell. The most commonly used RNA-guided DNA nucleases are derived from CRISPR systems, however, other RNA-guided DNA nucleases are also contemplated for use in the genome editing compositions and methods described herein. For instance, see U.S. Patent Publication No. 2015/0211023, incorporated herein by reference.

CRISPR systems that may be used in the practice of the invention vary greatly. CRISPR systems can be a type I, a type II, or a type III system. Non-limiting examples of suitable CRISPR proteins include Cas3, Cas4, Cas5, Cas5e (or CasD), Cas6, Cas6e, Cas6f, Cas7, Cas8a1, Cas8a2, Cas8b, Cas8c, Cas9, Cas10, Casl Od, CasF, CasG, CasH, Csy1, Csy2, Csy3, Cse1 (or CasA), Cse2 (or CasB), Cse3 (or CasE), Cse4 (or CasC), Cscl, Csc2, Csa5, Csn2, Csm2, Csm3, Csm4, Csm5, Csm6, Cmr1, Cmr3, Cmr4, Cmr5, Cmr6, Csb1, Csb2, Csb3,Csx17, Csx14, Csx10, Csx16, CsaX, Csx3, Cszl, Csx5, Csf1, Csf2, Csf3, Csf4, and Cul966.

In some embodiments, the RNA-guided DNA nuclease is a CRISPR nuclease derived from a type II CRISPR system (e.g., Cas9). The CRISPR nuclease may be derived from Streptococcus pyogenes, Streptococcus thermophilus, Streptococcus sp., Staphylococcus aureus, Neisseria meningitidis, Treponema denticola, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium Finegoldia magna, Natranaerobius thermophilus, Pelotomaculumthermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Acaryochloris marina, or any species which encodes a CRISPR nuclease with a known PAM sequence. CRISPR nucleases encoded by uncultured bacteria may also be used in the context of the invention. (See Burstein et al. Nature, 2017). Variants of CRIPSR proteins having known PAM sequences e.g., SpCas9 D1135E variant, SpCas9 VQR variant, SpCas9 EQR variant, or SpCas9 VRER variant may also be used in the context of the invention.

Thus, an RNA guided DNA nuclease of a CRISPR system, such as a Cas9 protein or modified Cas9 or homolog or ortholog of Cas9, or other RNA guided DNA nucleases belonging to other types of CRISPR systems, such as Cpf1 and its homologs and orthologs, may be used in the compositions of the present invention. Additional CRISPR nucleases may also be used, for example, the nucleases described in PCT International Application Publication Nos. WO2020/223514 and WO2020/223553, which are hereby incorporated by reference.

In certain embodiments, the CRIPSR nuclease may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some cases, the cell does not naturally produce Cas protein and is genetically engineered to produce a Cas protein.

In some embodiments, the CRISPR nuclease is Cpf1. Cpf1 is a single RNA-guided endonuclease which utilizes a T-rich protospacer-adjacent motif. Cpf1 cleaves DNA via a staggered DNA double-stranded break. Two Cpf1 enzymes from Acidaminococcus and Lachnospiraceae have been shown to carry out efficient genome-editing activity in human cells. (See Zetsche et al., 2015).

Thus, an RNA guided DNA nuclease of a Type II CRISPR System, such as a Cas9 protein or modified Cas9 or homologs, orthologues, or variants of Cas9, or other RNA guided DNA nucleases belonging to other types of CRISPR systems, such as Cpf1 and its homologs, orthologues, or variants, may be used in the present invention.

In some embodiments, the guide molecule comprises one or more chemical modifications which imparts a new or improved property (e.g., improved stability from degradation, improved hybridization energetics, or improved binding properties with an RNA guided DNA nuclease). Suitable chemical modifications include, but are not limited to: modified bases, modified sugar moieties, or modified inter-nucleoside linkages. Non-limiting examples of suitable chemical modifications include: 4-acetylcytidine, 5-(carboxyhydroxymethyl)uridine, 2′-O-methylcytidine, 5-carboxymethylaminomethyl-2-thiouridine, 5-carboxymethylaminomethyluridine, dihydrouridine, 2′-O-methylpseundouridine, “beta, D-galactosylquenosine”, 2′-O-methylguanosine, inosine, N6-isopentenyladenosine. 1-methyladenosine, 1-methylpseudouridine, 1-methylguanosine, 1-methylinosine, “2,2-dimethylguanosine”, 2-methyladenosine, 2-methylguanosine, 3-methylcytidine, 5-methylcytidine, N6-methyladenosine, 7-methylguanosine, 5-methylaminomethyluridine, 5-methoxyaminomethyl-2-thiouridine, “beta, D-mannosylqueuosine”, 5-methoxycarbonylmethyl-2-thiouridine, 5-methoxycarbonylmethyluridine, 5-methoxyuridine, 2-methylthio-N6-isopentenyladenosine, N-((9-beta-D-ribofuranosyl-2-methylthiopurine-6-yl)carbamoyl)threonine, N4(9-beta-D-ribofuranosylpurine-6-yl)N-methylcarbamoyl)threonine, uridine-5-oxyacetic acid-methylester, uridine-5-oxyacetic acid, wybutoxosine, queuosine, 2-thiocytidine, 5-methyl-2-thiouridine, 2-thiouridine, 4-thiouridine, N-((9-beta-D-ribofuranosylpurine-6-yl)-carbamoyl)threonine, 2′-O-methyl-2′-O-methyluridine, wybutosine, “3-(3-amino-3-carboxy-propyl)uridine, (acp3)u”, 2′-0-methyl (M), 3′-phosphorothioate (MS), 3′-thioPACE (MSP), pseudouridine, or 1-methyl pseudo-uridine. Each possibility represents a separate embodiment of the present invention.

Guide Sequences which Specifically Target a Mutant Allele

A given gene may contain thousands of SNPs. Utilizing a twenty-five base pair target window for targeting each SNP in a gene would require hundreds of thousands of guide sequences. Any given guide sequence when utilized to target a SNP may result in degradation of the guide sequence, limited activity, no activity, or off-target effects. Accordingly, suitable guide sequences are necessary for targeting a given gene. By the present invention, a novel set of guide sequences have been identified for knocking out expression of a mutated LRRK2 protein, inactivating a mutant LRRK2 gene allele, and treating monogenic Parkinson's disease.

The present disclosure provides guide sequences capable of specifically targeting a mutated allele for inactivation while leaving the functional allele unmodified. The guide sequences of the present invention are designed to, and are most likely to, specifically differentiate between a mutated allele and a functional allele. Of all possible guide sequences which target a mutated allele desired to be inactivated, the specific guide sequences disclosed herein are specifically effective to function with the disclosed embodiments.

Briefly, the guide sequences may have properties as follows: (1) target SNP/insertion/deletion/indel with a high prevalence in the general population, in a specific ethnic population or in a patient population is above 1% and the SNP/insertion/deletion/indel heterozygosity rate in the same population is above 1%; (2) target a location of a SNP/insertion/deletion/indel proximal to a portion of the gene e.g., within 5k bases of any portion of the gene, for example, a promoter, a UTR, an exon or an intron; and (3) target a mutant allele using an RNA molecule which targets a founder or common pathogenic mutations for the disease/gene. In some embodiments, the prevalence of the SNP/insertion/deletion/indel in the general population, in a specific ethnic population or in a patient population is above 1%, 2%, 3%, 4%, 5%, 6%, 7%, 8%, 9%, 10%, 11%, 12%, 13%, 14%, or 15% and the SNP/insertion/deletion/indel heterozygosity rate in the same population is above 1%, 2%, 3%, 4%, 5%, 6%, 7%, 8%, 9%, 10%, 11%, 12%, 13%, 14%, or 15%. Each possibility represents a separate embodiment and may be combined at will.

For each gene, according to SNP/insertion/deletion/indel any one of the following strategies may be used to deactivate the mutated allele: (1) Knockout strategy using one RNA molecule—one RNA molecule is utilized to direct a CRISPR nuclease to a mutated allele and create a double-strand break (DSB) leading to formation of a frameshift mutation in an exon or in a splice site region of the mutated allele; and (2) Excision of at least one coding exon or a complete knockout of a mutant LRRK2 allele using two RNA molecules, for example, a first RNA molecule targets a SNP position of an Intron 2 of the mutant LRRK2 allele and a second, non-discriminatory RNA molecule targets a sequence in Intron 1, preferably within 500 basepairs upstream to Exon 2, of the LRRK2 gene.

Based on the locations of identified SNPs/insertions/deletions/indels for each mutant allele, any one of, or a combination of, the above-mentioned methods to deactivate the mutant allele may be utilized.

In some embodiments of the present invention, an RNA molecule is used to target a pathogenic mutation within a mutant LRRK2 allele. In some embodiments of the present invention, an RNA molecule is used to target a SNP position. In some embodiments, the location of the SNP position is within a region of three kilobases upstream of the transcriptional start site to three kilobases downstream of the LRRK2 3′UTR.

Guide sequences of the present invention may: (1) target a heterozygous SNP for the targeted gene; (2) target a heterozygous SNP upstream or downstream of the gene; (3) target a SNP with a prevalence of the SNP/insertion/deletion/indel in the general population, in a specific ethnic population, or in a patient population above 1%; (4) have a guanine-cytosine content of greater than 30% and less than 85%; (5) have no repeat of seven or more thymine/uracil, guanine, cytosine, or adenine; and (6) have no additional target in the genome with zero mismatch with the same PAM sequence. Guide sequences of the present invention may satisfy any one of the above criteria and are most likely to differentiate between a mutated allele from its corresponding functional allele.

In some embodiments of the present invention, at least one nucleotide which differs between the mutated allele and the functional allele is upstream, downstream or within the sequence of the disease-causing mutation of the gene of interest. The at least one nucleotide which differs between the mutated allele and the functional allele may be within an exon or within an intron of the gene of interest. In some embodiments, the at least one nucleotide which differs between the mutated allele and the functional allele is within an exon of the gene of interest. In some embodiments, the at least one nucleotide which differs between the mutated allele and the functional allele is within an intron or the exon of the gene of interest, in close proximity to the splice site between the intron and the exon e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 nucleotides upstream or downstream to the splice site. Each possibility represents a separate embodiment.

In some embodiments, the at least one nucleotide is a single nucleotide polymorphism (SNP). In some embodiments, each of the nucleotide variants of the SNP may be expressed in the mutated allele. In some embodiments, the SNP may be a founder or common pathogenic mutation.

Guide sequences may target a SNP which has both (1) a high prevalence in the general population e.g., above 1% in the population; and (2) a high heterozygosity rate in the population, e.g., above 1%. Guide sequences may target a SNP that is globally distributed. A SNP may be a founder or common pathogenic mutation. In some embodiments, the prevalence in the general population is above 1%, 2%, 3%, 4%, 5%, 6%, 7%, 8%, 9%, 10%, 11%, 12%, 13%, 14%, or 15%. Each possibility represents a separate embodiment. In some embodiments, the heterozygosity rate in the population is above 1%, 2%, 3%, 4%, 5%, 6%, 7%, 8%, 9%, 10%, 11%, 12%, 13%, 14%, or 15%. Each possibility represents a separate embodiment.

In some embodiments, the at least one nucleotide which differs between the mutated allele and the functional allele is linked to/co-exists with the disease-causing mutation in high prevalence in a population. In such embodiments, “high prevalence” refers to at least 92%, 93%, 94%, 95%, 96%, 97%, 98%, 99%, or 100%. Each possibility represents a separate embodiment of the present invention. In one embodiment, the at least one nucleotide which differs between the mutated allele and the functional allele, is a disease-associated mutation. In some embodiments, the SNP is highly prevalent in the population. In such embodiments, “highly prevalent” refers to at least 10%, 11%, 12%, 13%, 14%, 15%, 20%, 30%, 40%, 50%, 60%, or 70% of a population. Each possibility represents a separate embodiment of the present invention.

### Delivery to Cells

The RNA molecule compositions described herein may be delivered to a target cell by any suitable means. RNA molecule compositions of the present invention may be targeted to any cell which contains and/or expresses a mutated allele, including any mammalian or plant cell. For example, in one embodiment the RNA molecule specifically targets a mutated LRRK2 allele and the target cell is an HSC. The delivery to the cell may be performed in-vitro, ex-vivo, or in-vivo. Further, the nucleic acid compositions described herein may be delivered as one or more of DNA molecules, RNA molecules, ribonucleoproteins (RNPs), nucleic acid vectors, or any combination thereof.

In some embodiments, any one of the compositions described herein is in the form of an RNP composition and is delivered to a cell ex-vivo. In some embodiments, the cell is a stem cell, a neuron, or a dopaminergic cell. The RNP composition may be delivered to the cell by any known ex-vivo delivery method, including but not limited to, electroporation, viral transduction, nanoparticle delivery, liposomes, etc. Additional detailed delivery methods are described throughout this section.

In some embodiments, the RNA molecule comprises a chemical modification. Non-limiting examples of suitable chemical modifications include 2′-0-methyl (M), 2′-0-methyl, 3′phosphorothioate (MS) or 2′-0-methyl, 3′thioPACE (MSP), pseudouridine, and 1-methyl pseudo-uridine. Each possibility represents a separate embodiment of the present invention.

Any suitable viral vector system may be used to deliver nucleic acid compositions e.g., the RNA molecule compositions of the subject invention. Conventional viral and non-viral based gene transfer methods can be used to introduce nucleic acids and target tissues. In certain embodiments, nucleic acids are administered for in vivo or ex vivo gene therapy uses. Non-viral vector delivery systems include naked nucleic acid, and nucleic acid complexed with a delivery vehicle such as a liposome or poloxamer. For a review of gene therapy procedures, see Anderson (1992); Nabel & Felgner (1993); Mitani & Caskey (1993); Dillon (1993); Miller (1992); Van Brunt (1988); Vigne (1995); Kremer & Perricaudet (1995); Haddada et al. (1995); and Yu et al. (1994).

Methods of non-viral delivery of nucleic acids and/or proteins include electroporation, lipofection, microinjection, biolistics, particle gun acceleration, virosomes, liposomes, immunoliposomes, lipid nanoparticles (LNPs), polycation or lipid:nucleic acid conjugates, artificial virions, and agent-enhanced uptake of nucleic acids or can be delivered to plant cells by bacteria or viruses (e.g., Agrobacterium, Rhizobium sp. NGR234, Sinorhizoboiummeliloti, Mesorhizobium loti, tobacco mosaic virus, potato virus X, cauliflower mosaic virus and cassava vein mosaic virus). (See, e.g., Chung et al., 2006). Sonoporation using, e.g., the Sonitron 2000 system (Rich-Mar), can also be used for delivery of nucleic acids. Cationic-lipid mediated delivery of proteins and/or nucleic acids is also contemplated as an in vivo, ex vivo, or in vitro delivery method. (See Zuris et al. (2015); see also Coelho et al. (2013); Judge et al. (2006); and Basha et al. (2011)).

Non-viral vectors, such as transposon-based systems e.g. recombinant Sleeping Beauty transposon systems or recombinant PiggyBac transposon systems, may also be delivered to a target cell and utilized for transposition of a polynucleotide sequence of a molecule of the composition or a polynucleotide sequence encoding a molecule of the composition in the target cell.

Additional exemplary nucleic acid delivery systems include those provided by Amaxa.RTM. Biosystems (Cologne, Germany), Maxcyte, Inc. (Rockville, Md.), BTX Molecular Delivery Systems (Holliston, Mass.) and Copernicus Therapeutics Inc., (see, e.g., U.S. Pat. No. 6,008,336). Lipofection is described in e.g., U.S. Pat. Nos. 5,049,386, 4,946,787; and 4,897,355, and lipofection reagents are sold commercially (e.g., Transfectam.™., Lipofectin.™. and Lipofectamine.™. RNAiMAX). Cationic and neutral lipids that are suitable for efficient receptor-recognition lipofection of polynucleotides include those disclosed in PCT International Publication Nos. WO/1991/017424 and WO/1991/016024. Delivery can be to cells (ex vivo administration) or target tissues (in vivo administration).

The preparation of lipid:nucleic acid complexes, including targeted liposomes such as immunolipid complexes, is well known to one of skill in the art (see, e.g., Crystal, Science (1995); Blaese et al., (1995); Behr et al., (1994); Remy et al. (1994); Gao and Huang (1995); Ahmad and Allen (1992); U.S. Pat. Nos. 4,186,183; 4,217,344; 4,235,871; 4,261,975; 4,485,054; 4,501,728; 4,774,085; 4,837,028; and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (See MacDiarmid et al., 2009).

The use of RNA or DNA viral based systems for viral mediated delivery of nucleic acids take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of nucleic acids include, but are not limited to, retroviral, lentivirus, adenoviral, adeno-associated, vaccinia and herpes simplex virus vectors for gene transfer.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (See, e.g., Buchschacher et al. (1992); Johann et al. (1992); Sommerfelt et al. (1990); Wilson et al. (1989); Miller et al. (1991); PCT International Publication No. WO/1994/026877A1).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (See Dunbar et al., 1995; Kohn et al., 1995; Malech et al., 1997). PA317/pLASN was the first therapeutic vector used in a gene therapy trial (Blaese et al., 1995). Transduction efficiencies of 50% or greater have been observed for MFG-S packaged vectors. (Ellem et al., (1997); Dranoff et al., 1997).

Packaging cells are used to form virus particles that are capable of infecting a host cell. Such cells include 293 cells, which package adenovirus, AAV, and Psi-2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additionally, AAV can be produced at clinical scale using baculovirus systems (see U.S. Pat. No. 7,479,554).

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al. (1995) reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravitreal, intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, optionally after selection for cells which have incorporated the vector. A non-limiting exemplary ex vivo approach may involve removal of tissue (e.g., peripheral blood, bone marrow, and spleen) from a patient for culture, nucleic acid transfer to the cultured cells (e.g., hematopoietic stem cells), followed by grafting the cells to a target tissue (e.g., bone marrow, and spleen) of the patient. In some embodiments, the stem cell or hematopoietic stem cell may be further treated with a viability enhancer.

Ex vivo cell transfection for diagnostics, research, or for gene therapy (e.g., via re-infusion of the transfected cells into the host organism) is well known to those of skill in the art. In a preferred embodiment, cells are isolated from the subject organism, transfected with a nucleic acid composition, and re-infused back into the subject organism (e.g., patient). Various cell types suitable for ex vivo transfection are well known to those of skill in the art (See, e.g., Freshney, “Culture of Animal Cells, A Manual of Basic Technique and Specialized Applications (6th edition, 2010) and the references cited therein for a discussion of how to isolate and culture cells from patients).

Suitable cells include, but are not limited to, eukaryotic cells and/or cell lines. Non-limiting examples of such cells or cell lines generated from such cells include COS, CHO (e.g., CHO—S, CHO-K1, CHO-DG44, CHO-DUXB11, CHO-DUKX, CHOK1SV), VERO, MDCK, WI38, V79, B14AF28-G3, BHK, HaK, NSO, SP2/0-Ag14, HeLa, HEK293 (e.g., HEK293-F, HEK293-H, HEK293-T), perC6 cells, any plant cell (differentiated or undifferentiated), as well as insect cells such as Spodopterafugiperda (Sf), or fungal cells such as Saccharomyces, Pichia and Schizosaccharomyces. In certain embodiments, the cell line is a CHO-K1, MDCK or HEK293 cell line. Additionally, primary cells may be isolated and used ex vivo for reintroduction into the subject to be treated following treatment with a guided nuclease system (e.g. CRISPR/Cas). Suitable primary cells include peripheral blood mononuclear cells (PBMC), and other blood cell subsets such as, but not limited to, CD4+ T cells or CD8+ T cells. Suitable cells also include stem cells such as, by way of example, embryonic stem cells, induced pluripotent stem cells, hematopoietic stem cells (CD34+), neuronal stem cells and mesenchymal stem cells.

In one embodiment, stem cells are used in ex vivo procedures for cell transfection and gene therapy. The advantage to using stem cells is that they can be differentiated into other cell types in vitro, or can be introduced into a mammal (such as the donor of the cells) where they will engraft in the bone marrow. Methods for differentiating CD34+ cells in vitro into clinically important immune cell types using cytokines such a GM-CSF, IFN-gamma, and TNF-alpha are known (as a non-limiting example see, Inaba et al., 1992).

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+(panB cells), GR-1 (granulocytes), and Tad (differentiated antigen presenting cells) (as a non-limiting example, see Inaba et al., 1992). Stem cells that have been modified may also be used in some embodiments.

Vectors (e.g., retroviruses, liposomes, etc.) containing therapeutic nucleic acid compositions can also be administered directly to an organism for transduction of cells in vivo. Administration is by any of the routes normally used for introducing a molecule into ultimate contact with blood or tissue cells including, but not limited to, injection, infusion, topical application (e.g., eye drops and cream) and electroporation. Suitable methods of administering such nucleic acids are available and well known to those of skill in the art, and, although more than one route can be used to administer a particular composition, a particular route can often provide a more immediate and more effective reaction than another route. According to some embodiments, the composition is delivered via IV injection.

Vectors suitable for introduction of transgenes into immune cells (e.g., T-cells) include non-integrating lentivirus vectors. See, e.g., U.S. Patent Publication No. 2009/0117617.

Pharmaceutically acceptable carriers are determined in part by the particular composition being administered, as well as by the particular method used to administer the composition. Accordingly, there is a wide variety of suitable formulations of pharmaceutical compositions available, as described below (See, e.g., Remington's Pharmaceutical Sciences, 17th ed., 1989).

In accordance with some embodiments, there is provided an RNA molecule which binds to/associates with and/or directs the RNA guided DNA nuclease to a sequence comprising at least one nucleotide which differs between a mutated allele and a functional allele (e.g., SNP) of a gene of interest (i.e., a sequence of the mutated allele which is not present in the functional allele). The sequence may be within the disease associated mutation. The sequence may be upstream or downstream to the disease associated mutation. Any sequence difference between the mutated allele and the functional allele may be targeted by an RNA molecule of the present invention to inactivate the mutant allele, or otherwise disable its dominant disease-causing effects, while preserving the activity of the functional allele.

The disclosed compositions and methods may also be used in the manufacture of a medicament for treating dominant genetic disorders in a patient.

### Mechanisms of Action for LRRK2 Knockout Methods

Without being bound by any theory or mechanism, the instant invention may be utilized to apply a CRISPR nuclease to process a mutated pathogenic LRRK2 allele and not a functional LRRK2 allele, such as to prevent expression of the mutated pathogenic allele or to produce a truncated non-pathogenic peptide from the mutated pathogenic allele, in order to prevent or treat monogenic Parkinson's disease. A specific guide sequence may be selected from Table 1 based on the targeted SNP position and the type of CRISPR nuclease used (e.g. according to a required PAM sequence). Notably, according to the Genome Aggregation Database (gnomAd) there are healthy individuals with loss of function mutations in one of their LRRK2 alleles. Therefore, there is no concern for haploinsufficiency upon knocking out a mutated LRRK2 allele.

The LRRK2 gene is located on chromosome 12 and encodes the Leucine-rich repeat serine/threonine-protein kinase 2 protein. One strategy to knockout a LRRK2 mutant allele is to target a pathogenic mutation position using one RNA molecule in order to mediate truncation or nonsense mediated decay (NMD) of the LRRK2 mutant allele. As a non-limiting example, a frameshift in a mutated LRRK2 allele may be introduced by utilizing one RNA molecule to target a pathogenic mutation or SNP position in a coding exon of the mutated LRRK2 allele (e.g. rs10878245 in Exon 5, rs1427263 in Exon 34 and rs10878405 in Exon 43) in order to mediate a double-strand break, which leads to generation of a frameshift mutation and expression of a truncated protein or nonsense mediated decay (NMD) of its transcripts.

Alternatively, a LRRK2 mutant allele may be knocked-out by an excision strategy that utilizes two RNA molecules. In such strategies, SNP positions within three kilobases upstream to the transcription start site and three kilobases downstream of the 3′UTR of the LRRK2 gene may be targeted.

In one excision strategy approach, Exon 2 of a mutant LRRK2 allele is excised by utilizing a first RNA molecule to target a SNP position in Intron 2 of the mutant LRRK2 allele and utilizing a second non-discriminatory RNA molecule to target a region in Intron 1, of the LRRK2 gene. In some embodiments of this approach, the target region is within 500 bps of Exon 2.

In another excision strategy approach, Exon 3 of a mutant LRRK2 allele is excised by utilizing a first RNA molecule to target a SNP position in Intron 4 and utilizing a second non-discriminatory RNA molecule to target a region in Intron 3. In some embodiments of this approach, the target region is within 500 bps of Exon 4.

In another excision strategy approach, the 3′UTR of a mutant LRRK2 allele is excised by utilizing a first RNA molecule to target a SNP position in Intron 49 and utilizing a second non-discriminatory RNA molecule to target a region within 500 base pairs downstream to Exon 51. In some embodiments of this approach, the target region is within 500 bps of Exon 4.

Alternatively, the 3′UTR of a mutant LRRK2 allele is excised by utilizing a first RNA molecule to target a SNP position downstream of Exon 51 and utilizing a second non-discriminatory RNA molecule to target a region in Intron 50 or Intron 49, preferably within 500 base pairs upstream to Exon 50.

In another excision strategy approach, a promoter region of a mutant LRRK2 allele is excised by utilizing a first RNA molecule to target a SNP with 3000 base pairs of the transcriptional start site and utilizing a second non-discriminatory RNA molecule to target a region in Intron 1 or Intron 2, preferably within 500 base pairs downstream to Exon 2.

Examples of RNA Guide Sequences which Specifically Target Mutated Alleles of LRRK2 Gene

Although a large number of guide sequences can be designed to target a mutated allele, the nucleotide sequences described in Table 1 identified by SEQ ID NOs: 1-103,606 below were specifically selected to effectively implement the methods set forth herein and to effectively discriminate between alleles.

Table 1 shows guide sequences designed for use as described in the embodiments above to associate with different SNPs or pathogenic mutations within a sequence of a mutated LRRK2 allele. Each engineered guide molecule is further designed such as to associate with a target genomic DNA sequence of interest that lies next to a protospacer adjacent motif (PAM), e.g., a PAM matching the sequence NGG or NAG, where “N” is any nucleobase. The guide sequences were designed to work in conjunction with one or more different CRISPR nucleases, including, but not limited to, e.g. SpCas9WT (PAM SEQ: NGG), SpCas9.VQR.1 (PAM SEQ: NGAN), SpCas9.VQR.2 (PAM SEQ: NGNG), SpCas9.EQR (PAM SEQ: NGAG), SpCas9.VRER (PAM SEQ: NGCG), SaCas9WT (PAM SEQ: NNGRRT), SpRY (PAM SEQ: NRN or NYN), NmCas9WT (PAM SEQ: NNNNGATT), Cpf1 (PAM SEQ: TTTV), or JeCas9WT (PAM SEQ: NNNVRYM). RNA molecules of the present invention are each designed to form complexes in conjunction with one or more different CRISPR nucleases and designed to target polynucleotide sequences of interest utilizing one or more different PAM sequences respective to the CRISPR nuclease utilized.

TABLE 1
Guide sequences designed to associate with specific LRRK2 gene targets

| | SEQ ID NOS: | | |
|---|---|---|---|
| | of 20 base | SEQ ID NOs: of | SEQ ID NOs: of |
| Target | guides | 21 base guides | 22 base guides |
| 12:40225041_C_T | 1-46 | 47-96 | 97-148 |
| 12:40225058_C_A | 149-196 | 197-246 | 247-298 |
| 12:40225088_G_A | 299-345 | 346-395 | 396-447 |
| 12:40225159_G_A | 448-495 | 496-545 | 546-597 |
| 12:40225233_G_C | 598-645 | 646-695 | 696-747 |
| 12:40225558_C_T | 748-791 | 792-833 | 834-877 |
| 12:40225618_T_C | 878-921 | 922-971 | 972-1023 |
| 12:40225628_G_A | 1024-1070 | 1071-1120 | 1121-1172 |
| 12:40225631_T_C | 1024, 1035, | 1072, 1083, | 1121, 1123, |
| | 1049, 1055, | 1104, 1116, | 1168, 1171, |
| | 1173-1215 | 1216-1261 | 1262-1309 |
| 12:40232303_A_C | 1310-1356 | 1357-1406 | 1407-1458 |
| 12:40232345_G_C | 1459-1505 | 1506-1555 | 1556-1607 |
| 12:40232380_A_C | 1608-1652 | 1653-1698 | 1699-1746 |
| 12:40232385_T_C | 1747-1788 | 1789-1828 | 1829-1870 |
| 12:40235634_T_C | 1871-1912 | 1913-1945 | 1946-1988 |
| 12:40235646_C_G | 1989-2018 | 2019-2041 | 2042-2068 |
| 12:40235660_A_G | 2069-2113 | 2114-2160 | 2161-2209 |
| 12:40238078_A_G | 2210-2257 | 2258-2307 | 2308-2359 |
| 12:40240517_C_T | 2360-2367 | 2368-2373 | 2374-2381 |
| 12:40240531_T_C | 2382-2400 | 2401-2415 | 2416-2432 |
| 12:40240594_G_C | 2433-2479 | 2480-2529 | 2530-2581 |
| 12:40240602_T_C | 2582-2629 | 2630-2679 | 2680-2731 |
| 12:40243551_CAAT_C | 2732-2767 | 2768-2808 | 2809-2852 |
| 12:40243556_A_T | 2738, 2752, | 2776, 2784, | 2818, 2833, |
| | 2760, 2763, | 2791, 2793, | 2835, 2839, |
| | 2853-2885 | 2803-2804, | 2846-2847, |
| | | 2886-2918 | 2851, 2919-2953 |
| 12:40249822_T_G | 2954-2973 | 2974-2982 | 2983-2997 |
| 12:40249843_C_G | 2998-3019 | 3020-3039 | 3040-3065 |
| 12:40249854_C_T | 3066-3098 | 3099-3126 | 3127-3158 |
| 12:40249881_T_C | 3159-3205 | 3206-3255 | 3256-3307 |
| 12:40249908_A_G | 3308-3355 | 3356-3405 | 3406-3457 |
| 12:40251273_G_A | 3458-3475 | 3476-3480 | 3481-3490 |
| 12:40251303_G_A | 3491-3526 | 3527-3563 | 3564-3603 |
| 12:40251361_A_G | 3604-3651 | 3652-3701 | 3702-3753 |
| 12:40251366_C_A | 3754-3800 | 3801-3850 | 3851-3902 |
| 12:40251369_G_A | 3761, 3775, | 3803, 3808, | 3853, 3868, |
| | 3799, 3903- | 3818, 3850, | 3890, 3894, |
| | 3945 | 3946-3991 | 3992-4039 |
| 12:40251495_C_T | 4040-4065 | 4066-4083 | 4084-4108 |
| 12:40252906_A_G | 4109-4135 | 4136-4163 | 4164-4195 |
| 12:40252929_G_A | 4196-4243 | 4244-4293 | 4294-4345 |
| 12:40252939_C_T | 4346-4392 | 4393-4442 | 4443-4494 |
| 12:40252952_T_G | 4495-4541 | 4542-4591 | 4592-4643 |
| 12:40252958_A_C | 4644-4691 | 4692-4741 | 4742-4793 |
| 12:40252968_G_A | 4794-4834 | 4835-4874 | 4875-4920 |
| 12:40252974_C_T | 4921-4966 | 4967-5012 | 5013-5063 |
| 12:40252978_C_T | 4944, 4964, | 4998, 5004, | 5020, 5060, |
| | 5064-5108 | 5109-5156 | 5157-5206 |
| 12:40252984_C_T | 5207-5253 | 5254-5303 | 5304-5355 |
| 12:40252988_T_C | 5236, 5244, | 5262, 5300, | 5306, 5338, |
| | 5356-5400 | 5401-5448 | 5449-5498 |
| 12:40253016_G_A | 5499-5537 | 5538-5567 | 5568-5605 |
| 12:40257311_A_G | 5606-5648 | 5649-5698 | 5699-5750 |
| 12:40257341_G_T | 5751-5796 | 5797-5838 | 5839-5884 |
| 12:40259507_A_G | 5885-5932 | 5933-5982 | 5983-6034 |
| 12:40259525_A_T | 6035-6074 | 6075-6112 | 6113-6154 |
| 12:40259534_G_T | 6155-6186 | 6187-6211 | 6212-6243 |
| 12:40259578_G_A | 6244-6277 | 6278-6311 | 6312-6347 |
| 12:40263806_A_G | 6348-6394 | 6395-6442 | 6443-6493 |
| 12:40263875_A_G | 6494-6538 | 6539-6579 | 6580-6626 |
| 12:40263900_G_A | 6627-6662 | 6663-6690 | 6691-6720 |
| 12:40263907_T_C | 6721-6756 | 6757-6788 | 6789-6824 |
| 12:40274646_CC_C | 6825-6858 | 6859-6879 | 6880-6908 |
| 12:40274881_T_C | 6909-6953 | 6954-6993 | 6994-7040 |
| 12:40274899_A_G | 7041-7084 | 7085-7124 | 7125-7169 |
| 12:40274917_G_C | 7170-7216 | 7217-7262 | 7263-7311 |
| 12:40277933_T_C | 7312-7359 | 7360-7408 | 7409-7460 |
| 12:40277968_A_C | 7461-7500 | 7501-7523 | 7524-7559 |
| 12:40277971_A_G | 7469, 7471, | 7503, 7510, | 7527, 7543, |
| | 7481, 7498, | 7515, 7518, | 7545, 7551, |
| | 7560-7592 | 7593-7613 | 7614-7645 |
| 12:40278025_T_A | 7646-7693 | 7694-7742 | 7743-7794 |
| 12:40278154_A_G | 7795-7831 | 7832-7865 | 7866-7903 |
| 12:40278167_C_T | 7904-7950 | 7951-7998 | 7999-8049 |
| 12:40283881_G_A | 8050-8073 | 8074-8099 | 8100-8127 |
| 12:40283915_T_C | 8128-8174 | 8175-8224 | 8225-8276 |
| 12:40283947_C_T | 8277-8324 | 8325-8374 | 8375-8426 |
| 12:40283966_G_A | 8427-8473 | 8474-8523 | 8524-8575 |
| 12:40283985_C_A | 8576-8622 | 8623-8672 | 8673-8724 |
| 12:40283990_T_C | 8725-8772 | 8773-8822 | 8823-8874 |
| 12:40284061_A_G | 8875-8921 | 8922-8967 | 8968-9017 |
| 12:40284076_C_A | 9018-9056 | 9057-9088 | 9089-9126 |
| 12:40284139_T_C | 9127-9164 | 9165-9193 | 9194-9226 |
| 12:40287363_C_A | 9227-9266 | 9267-9300 | 9301-9338 |
| 12:40287438_A_G | 9339-9385 | 9386-9435 | 9436-9487 |
| 12:40287461_A_G | 9488-9535 | 9536-9581 | 9582-9631 |
| 12:40293555_CTC_CCT | 9632-9671 | 9672-9711 | 9712-9753 |
| 12:40293607_C_T | 9754-9798 | 9799-9842 | 9843-9888 |
| 12:40293613_G_A | 9889-9936 | 9937-9986 | 9987-10038 |
| 12:40293624_G_C | 10039-10086 | 10087-10136 | 10137-10188 |
| 12:40293626_G_A | 10060, 10072, | 10109, 10119, | 10150, 10170, |
| | 10074, 10080, | 10124, 10130, | 10175, 10180, |
| | 10083-10084, | 10133, 10136, | 10185, 10188, |
| | 10189-10230 | 10231-10274 | 10275-10320 |
| 12:40293644_A_G | 10321-10368 | 10369-10418 | 10419-10470 |
| 12:40294854_T_G | 10471-10513 | 10514-10552 | 10553-10596 |
| 12:40294862_T_C | 10597-10639 | 10640-10679 | 10680-10724 |
| 12:40294866_G_T | 10622, 10628, | 10644, 10747- | 10681, 10758- |
| | 10725-10746 | 10757 | 10774 |
| 12:40294878_C_T | 10775-10789 | 10790-10794 | 10795-10802 |
| 12:40295448_A_G | 10803-10849 | 10850-10899 | 10900-10951 |
| 12:40295463_A_G | 10952-10998 | 10999-11048 | 11049-11100 |
| 12:40295466_G_A | 10955, 10965, | 11002, 11016, | 11067, 11076, |
| | 10991, 11101- | 11025, 11041, | 11091, 11095, |
| | 11144 | 11145-11190 | 11191-11238 |
| 12:40295535_A_T | 11239-11277 | 11278-11315 | 11316-11359 |
| 12:40295566_A_G | 11360-11407 | 11408-11457 | 11458-11509 |
| 12:40295574_A_C | 11510-11557 | 11558-11607 | 11608-11659 |
| 12:40295581_G_T | 11660-11706 | 11707-11745 | 11746-11791 |
| 12:40298330_A_G | 11792-11836 | 11837-11880 | 11881-11929 |
| 12:40298333_C_T | 11826, 11831, | 11847, 11864, | 11885, 11894, |
| | 11833, 11930- | 11876, 11957- | 11911, 11924, |
| | 11956 | 11972 | 11973-11994 |
| 12:40298346_G_A | 11995-12042 | 12043-12088 | 12089-12138 |
| 12:40298433_C_G | 12139-12186 | 12187-12236 | 12237-12288 |
| 12:40298479_G_T | 12289-12335 | 12336-12382 | 12383-12434 |
| 12:40298488_A_G | 12435-12481 | 12482-12530 | 12531-12582 |
| 12:40299125_A_G | 12583-12622 | 12623-12659 | 12660-12703 |
| 12:40299190_C_T | 12704-12751 | 12752-12800 | 12801-12852 |
| 12:40299196_A_C | 12853-12900 | 12901-12950 | 12951-13002 |
| 12:40299212_G_A | 13003-13049 | 13050-13099 | 13100-13151 |
| 12:40299255_T_C | 13152-13199 | 13200-13249 | 13250-13300 |
| 12:40302866_A_G | 13301-13347 | 13348-13384 | 13385-13427 |
| 12:40303952_C_A | 13428-13471 | 13472-13517 | 13518-13566 |
| 12:40303972_C_G | 13567-13613 | 13614-13663 | 13664-13715 |
| 12:40304000_G_A | 13716-13762 | 13763-13811 | 13812-13863 |
| 12:40304004_A_G | 13758, 13760, | 13774, 13787, | 13836, 13851, |
| | 13864-13909 | 13910-13956 | 13957-14006 |
| 12:40304035_A_C | 14007-14049 | 14050-14099 | 14100-14151 |
| 12:40304040_G_C | 14152-14193 | 14194-14227 | 14228-14272 |
| 12:40304040_G_T | 14152-14153, | 14194, 14202- | 14235, 14240- |
| | 14157, 14162, | 14203, 14208, | 14241, 14248, |
| | 14166, 14169, | 14215, 14219- | 14257, 14262, |
| | 14182, 14185, | 14220, 14226, | 14264, 14270, |
| | 14273-14291 | 14292-14308 | 14272, 14309- |
| | | | 14331 |
| 12:40304078_G_A | 14332-14352 | 14353-14370 | 14371-14392 |
| 12:40304141_C_T | 14393-14430 | 14431-14455 | 14456-14488 |
| 12:40305776_C_T | 14489-14505 | 14506-14522 | 14523-14541 |
| 12:40305781_T_A | 14542-14568 | 14569-14594 | 14595-14622 |
| 12:40305791_C_G | 14623-14666 | 14667-14711 | 14712-14758 |
| 12:40305871_G_A | 14759-14790 | 14791-14816 | 14817-14849 |
| 12:40305942_G_A | 14850-14868 | 14869-14882 | 14883-14900 |
| 12:40305946_T_A | 14901-14930 | 14873, 14931- | 14883, 14964- |
| | | 14963 | 14998 |
| 12:40308467_G_T | 14999-15018 | 15019-15034 | 15035-15053 |
| 12:40308476_A_G | 15054-15100 | 15101-15145 | 15146-15193 |
| 12:40308481_G_A | 15194-15230 | 15231-15266 | 15267-15309 |
| 12:40308543_GG_G | 15310-15355 | 15356-15403 | 15404-15453 |
| 12:40308632_C_A | 15454-15501 | 15502-15550 | 15551-15602 |
| 12:40308672_G_A | 15603-15649 | 15650-15699 | 15700-15751 |
| 12:40309174_G_A | 15752-15798 | 15799-15848 | 15849-15900 |
| 12:40309206_C_T | 15901-15946 | 15947-15996 | 15997-16048 |
| 12:40309225_A_C | 16049-16094 | 16095-16134 | 16135-16182 |
| 12:40309226_A_G | 16058, 16060, | 16102, 16117- | 16145-16146, |
| | 16066, 16075, | 16119, 16127, | 16162-16163, |
| | 16077, 16084, | 16129, 16223- | 16174-16176, |
| | 16088, 16183- | 16256 | 16257-16298 |
| | 16222 | | |
| 12:40309230_A_G | 16199, 16217, | 16231, 16237, | 16260, 16284, |
| | 16299-16343 | 16344-16382 | 16383-16429 |
| 12:40309236_G_A | 16430-16445 | 16446-16453 | 16454-16465 |
| 12:40310434_C_A | 16466-16510 | 16511-16560 | 16561-16612 |
| 12:40310434_C_G | 16469, 16476- | 16514, 16518, | 16564, 16568, |
| | 16477, 16500, | 16522, 16537, | 16572, 16587- |
| | 16502, 16506, | 16545, 16548, | 16588, 16596- |
| | 16509-16510, | 16550, 16557, | 16597, 16600, |
| | 16613-16649 | 16559-16560, | 16611-16612, |
| | | 16650-16689 | 16690-16731 |
| 12:40310434_C_T | 16469, 16476- | 16514, 16518, | 16564, 16568, |
| | 16477, 16500, | 16522, 16537, | 16572, 16587- |
| | 16502, 16506, | 16545, 16548, | 16588, 16596- |
| | 16509-16510, | 16550, 16557, | 16597, 16600, |
| | 16732-16768 | 16559-16560, | 16611-16612, |
| | | 16769-16808 | 16809-16850 |
| 12:40310435_G_A | 16469, 16476, | 16514, 16518, | 16564, 16568, |
| | 16502, 16506, | 16522, 16537, | 16587-16588, |
| | 16509-16510, | 16548, 16557, | 16597, 16600, |
| | 16851-16889 | 16559-16560, | 16611-16612, |
| | | 16890-16931 | 16932-16975 |
| 12:40310436_C_T | 16469, 16476, | 16514, 16518, | 16568, 16587- |
| | 16506, 16509, | 16537, 16557, | 16588, 16600, |
| | 16856, 16882, | 16559-16560, | 16611-16612, |
| | 16976-17015 | 16913, 16929, | 16949, 16974, |
| | | 17016-17057 | 17058-17101 |
| 12:40310437_G_C | 16469, 16506, | 16518, 16537, | 16568, 16588, |
| | 16856, 16882, | 16557, 16559, | 16611-16612, |
| | 16999, 17013, | 16913, 16929, | 16949, 16974, |
| | 17102-17141 | 17033, 17049, | 17063, 17099, |
| | | 17142-17183 | 17184-17227 |
| 12:40310440_T_G | 16999, 17013, | 17033, 17049, | 17063, 17099, |
| | 17119, 17134, | 17147, 17181, | 17184, 17219, |
| | 17228-17271 | 17272-17317 | 17318-17365 |
| 12:40310450_C_T | 17366-17413 | 17414-17463 | 17464-17515 |
| 12:40310461_G_A | 17516-17561 | 17562-17611 | 17612-17663 |
| 12:40310471_A_G | 17664-17707 | 17708-17757 | 17758-17809 |
| 12:40310476_GAT_G | 17810-17856 | 17857-17905 | 17906-17956 |
| 12:40310515_A_G | 17957-18003 | 18004-18053 | 18054-18105 |
| 12:40310530_C_T | 18106-18153 | 18154-18203 | 18204-18255 |
| 12:40310546_G_A | 18256-18303 | 18304-18353 | 18354-18405 |
| 12:40310554_G_A | 18406-18453 | 18454-18503 | 18504-18555 |
| 12:40310561_G_A | 18556-18602 | 18603-18652 | 18653-18704 |
| 12:40314043_G_A | 18705-18730 | 18731-18745 | 18746-18762 |
| 12:40314059_C_T | 18763-18799 | 18800-18834 | 18835-18876 |
| 12:40314062_A_G | 18783-18784, | 18813, 18819, | 18849, 18852, |
| | 18788, 18796, | 18831, 18833, | 18875-18876, |
| | 18877-18918 | 18919-18960 | 18961-19006 |
| 12:40314101_C_A | 19007-19029 | 19030-19049 | 19050-19077 |
| 12:40315266_T_A | 19078-19123 | 19124-19173 | 19174-19225 |
| 12:40319979_A_T | 19226-19262 | 19263-19289 | 19290-19323 |
| 12:40319998_T_C | 19324-19369 | 19370-19418 | 19419-19470 |
| 12:40320043_G_A | 19471-19515 | 19516-19557 | 19558-19604 |
| 12:40320054_G_C | 19605-19634 | 19635-19658 | 19659-19686 |
| 12:40320090_A_G | 19687-19726 | 19727-19765 | 19766-19808 |
| 12:40320094_A_G | 19712, 19809- | 19739, 19853- | 19793, 19897- |
| | 19852 | 19896 | 19942 |
| 12:40320097_T_C | 19811, 19816, | 19867, 19888, | 19920, 19934, |
| | 19825, 19841, | 19986-20030 | 20031-20077 |
| | 19943-19985 | | |
| 12:40320119_A_G | 20078-20123 | 20124-20167 | 20168-20215 |
| 12:40321114_A_G | 20216-20259 | 20260-20303 | 20304-20353 |
| 12:40321123_T_C | 20354-20396 | 20397-20438 | 20439-20482 |
| 12:40322037_C_T | 20483-20526 | 20527-20566 | 20567-20612 |
| 12:40322038_G_A | 20484-20485, | 20528, 20533, | 20570, 20573, |
| | 20490, 20492, | 20535, 20550, | 20579, 20596, |
| | 20509, 20518, | 20650-20687 | 20688-20731 |
| | 20520, 20613- | | |
| | 20649 | | |
| 12:40322047_G_A | 20732-20774 | 20775-20824 | 20825-20876 |
| 12:40322047_G_T | 20741, 20746, | 20784, 20789, | 20839, 20841, |
| | 20748, 20762, | 20791, 20793- | 20844, 20847, |
| | 20774, 20877- | 20794, 20803, | 20852, 20855, |
| | 20914 | 20809-20810, | 20861-20862, |
| | | 20813, 20819, | 20865, 20871, |
| | | 20915-20954 | 20955-20996 |
| 12:40322134_A_G | 20997-21043 | 21044-21090 | 21091-21139 |
| 12:40322137_A_C | 21006, 21030, | 21074, 21078, | 21120-21121, |
| | 21043, 21140- | 21181-21223 | 21224-21268 |
| | 21180 | | |
| 12:40322364_T_C | 21269-21314 | 21315-21364 | 21365-21416 |
| 12:40322386_G_T | 21417-21463 | 21464-21511 | 21512-21563 |
| 12:40322468_C_A | 21564-21579 | 21580 | 21581-21583 |
| 12:40323244_C_G | 21584-21623 | 21624-21661 | 21662-21701 |
| 12:40323255_A_G | 21702-21729 | 21730-21752 | 21753-21779 |
| 12:40323256_T_C | 21708, 21712, | 21743-21744, | 21768, 21773, |
| | 21716, 21720- | 21746, 21749, | 21776-21778, |
| | 21721, 21723, | 21751, 21801- | 21818-21838 |
| | 21726, 21780- | 21817 | |
| | 21800 | | |
| 12:40323260_G_T | 21797, 21839- | 21810, 21847- | 21832, 21851- |
| | 21846 | 21850 | 21857 |
| 12:40323270_G_T | 21858-21870 | 21871-21878 | 21879-21890 |
| 12:40323297_T_C | 21891-21938 | 21939-21985 | 21986-22035 |
| 12:40328416_G_C | 22036-22071 | 22072-22105 | 22106-22141 |
| 12:40335008_A_T | 22142-22189 | 22190-22239 | 22240-22291 |
| 12:40335031_G_A | 22292-22338 | 22339-22388 | 22389-22440 |
| 12:40335036_C_T | 22441-22488 | 22489-22538 | 22539-22590 |
| 12:40335037_G_A | 22447, 22463, | 22495, 22514, | 22545, 22567, |
| | 22466, 22470- | 22516, 22519- | 22571, 22573, |
| | 22471, 22475, | 22520, 22524, | 22576, 22585, |
| | 22484-22485, | 22533, 22537, | 22589-22590, |
| | 22591-22630 | 22631-22672 | 22673-22716 |
| 12:40335079_G_T | 22717-22764 | 22765-22814 | 22815-22866 |
| 12:40340349_C_G | 22867-22914 | 22915-22963 | 22964-23015 |
| 12:40340361_T_C | 23016-23061 | 23062-23111 | 23112-23163 |
| 12:40340380_T_C | 23164-23209 | 23210-23259 | 23260-23310 |
| 12:40340400_G_A | 23311-23354 | 23355-23403 | 23404-23455 |
| 12:40340404_T_C | 23352-23353, | 23368-23369, | 23426-23427, |
| | 23456-23499 | 23500-23547 | 23548-23597 |
| 12:40340432_A_G | 23598-23644 | 23645-23694 | 23695-23746 |
| 12:40340436_A_T | 23599, 23628, | 23672, 23677, | 23699, 23707, |
| | 23747-23792 | 23793-23840 | 23841-23890 |
| 12:40346818_T_C | 23891-23932 | 23933-23966 | 23967-24008 |
| 12:40346826_ACTACT_A | 24009-24028 | 24029-24036 | 24037-24048 |
| 12:40346884_A_G | 24049-24091 | 24092-24133 | 24134-24177 |
| 12:40348402_G_GTGT | 24178-24191 | , | 24192-24193 |
| 12:40348475_A_G | 24194-24232 | 24233-24262 | 24263-24294 |
| 12:40348484_C_T | 24295-24322 | 24323-24346 | 24347-24376 |
| 12:40351537_A_G | 24377-24418 | 24419-24459 | 24460-24505 |
| 12:40351572_T_A | 24506-24551 | 24552-24597 | 24598-24645 |
| 12:40351579_C_T | 24646-24691 | 24692-24737 | 24738-24786 |
| 12:40351585_G_A | 24787-24820 | 24821-24854 | 24855-24890 |
| 12:40351605_G_C | 24891-24911 | 24912-24924 | 24925-24941 |
| 12:40351623_G_T | 24942-24986 | 24987-25028 | 25029-25076 |
| 12:40351667_C_A | 25077-25124 | 25125-25174 | 25175-25226 |
| 12:40351680_G_C | 25227-25273 | 25274-25323 | 25324-25375 |
| 12:40351720_G_C | 25376-25419 | 25420-25454 | 25455-25497 |
| 12:40351723_A_G | 25408, 25417, | 25429, 25454, | 25467, 25469, |
| | 25419, 25498- | 25542-25586 | 25587-25634 |
| | 25541 | | |
| 12:40354304_T_C | 25635-25678 | 25679-25720 | 25721-25766 |
| 12:40354305_G_T | 25636, 25642, | 25685, 25796- | 25722, 25740, |
| | 25659, 25675, | 25821 | 25748, 25822- |
| | 25677, 25767- | | 25853 |
| | 25795 | | |
| 12:40354410_G_A | 25854-25901 | 25902-25951 | 25952-26003 |
| 12:40356126_A_T | 26004-26011 | 26012-26019 | 26020-26029 |
| 12:40356145_C_T | 26030-26073 | 26074-26117 | 26118-26163 |
| 12:40359345_C_T | 26164-26194 | 26195-26222 | 26223-26258 |
| 12:40359422_A_G | 26259-26305 | 26306-26353 | 26354-26404 |
| 12:40363435_C_T | 26405-26451 | 26452-26491 | 26492-26539 |
| 12:40363440_C_T | 26540-26586 | 26587-26632 | 26633-26684 |
| 12:40363541_G_A | 26685-26727 | 26728-26769 | 26770-26815 |
| 12:40364843_G_A | 26816-26856 | 26857-26892 | 26893-26936 |
| 12:40364845_G_GGT | 26937-26975 | 26976-27013 | 26904, 27014- |
| | | | 27055 |
| 12:40364884_G_A | 27056-27080 | 27081-27097 | 27098-27122 |
| 12:40364927_A_T | 27123-27169 | 27170-27219 | 27220-27271 |
| 12:40364960_A_G | 27272-27318 | 27319-27368 | 27369-27420 |
| 12:40365044_C_T | 27421-27468 | 27469-27518 | 27519-27570 |
| 12:40367012_T_A | 27571-27614 | 27615-27646 | 27647-27685 |
| 12:40367050_A_G | 27686-27733 | 27734-27783 | 27784-27835 |
| 12:40367648_AC_A | 27836-27863 | 27864-27887 | 27888-27913 |
| 12:40367656_G_T | 27914-27954 | 27955-27990 | 27991-28030 |
| 12:40367754_T_C | 28031-28077 | 28078-28127 | 28128-28179 |
| 12:40367849_G_A | 28180-28216 | 28217-28244 | 28245-28278 |
| 12:40367888_A_C | 28279-28304 | 28305-28328 | 28329-28354 |
| 12:40367988_C_T | 28355-28364 | , | , |
| 12:40368042_A_G | 28365-28394 | 28395-28417 | 28418-28444 |
| 12:40368073_A_C | 28445-28476 | 28477-28491 | 28492-28514 |
| 12:40368170_C_T | 28515-28547 | 28548-28566 | 28567-28593 |
| 12:40368262_C_T | , | 28594 | 28595-28596 |
| 12:40368380_T_A | 28597-28602 | 28603 | 28604-28605 |
| 12:40368486_G_T | 28606-28615 | 28616-28620 | 28621-28627 |
| 12:40368516_C_A | 28628-28661 | 28662-28690 | 28691-28724 |
| 12:40368534_T_G | 28725-28745 | 28746-28760 | 28761-28782 |
| 12:40368673_A_C | 28783-28826 | 28827-28871 | 28872-28918 |
| 12:40368736_C_A | 28919-28956 | 28957-28988 | 28989-29024 |
| 12:40368783_G_T | 29025-29062 | 29063-29096 | 29097-29136 |
| 12:40368800_T_C | 29137-29169 | 29170-29203 | 29204-29241 |
| 12:40368807_A_G | 29242-29276 | 29277-29311 | 29312-29350 |
| 12:40368834_A_G | 29351-29368 | 29369-29386 | 29387-29407 |
| 12:40368860_C_T | 29408-29447 | 29448-29481 | 29482-29517 |
| 12:40368905_A_T | 29518-29551 | 29552-29583 | 29584-29619 |
| 12:40368930_C_T | 29620-29652 | 29653-29676 | 29677-29708 |
| 12:40368947_C_T | 29709-29753 | 29754-29795 | 29796-29842 |
| 12:40368951_C_T | 29752, 29843- | 29771, 29876- | 29804, 29909- |
| | 29875 | 29908 | 29945 |
| 12:40369044_T_C | 29946-29948 | 29949 | 29950-29951 |
| 12:40369153_T_C | 29952-29974 | 29975-29988 | 29989-30006 |
| 12:40369167_G_T | 30007-30041 | 30042-30070 | 30071-30103 |
| 12:40222364_G_A | 30104-30107 | 30108 | 30109-30111 |
| rs1388598_REF | | | |
| 12:40222364_G_A | 30107 | , | , |
| rs1388598_SNP | | | |
| 12:40222612_A_G | 30112-30145 | 30146-30171 | 30172-30203 |
| rs28365207_REF | | | |
| 12:40222612_A_G | 30128, 30136- | 30156, 30164- | 30184, 30194, |
| rs28365207_SNP | 30138, 30145, | 30165, 30170- | 30201-30203, |
| | 30204-30236 | 30171, 30237- | 30270-30304 |
| | | 30269 | |
| 12:40223400_T_C | 30305 | , | 30306 |
| rs7294619_REF | | | |
| 12:40223400_T_C | 30305, 30307- | 30312 | 30306, 30313- |
| rs7294619_SNP | 30311 | | 30315 |
| 12:40223890_G_C | 30316-30363 | 30364-30413 | 30414-30465 |
| rs10878226_REF | | | |
| 12:40223890_G_C | 30320, 30330, | 30364, 30369, | 30414, 30419, |
| rs10878226_SNP | 30337, 30343- | 30374, 30387, | 30424, 30437, |
| | 30344, 30349, | 30393-30394, | 30441, 30445- |
| | 30352, 30357- | 30399, 30407- | 30446, 30459- |
| | 30358, 30360, | 30408, 30410, | 30460, 30462, |
| | 30466-30503 | 30504-30543 | 30544-30585 |
| 12:40226614_A_T | 30586-30627 | 30628-30659 | 30660-30697 |
| rs1491943_REF | | | |
| 12:40226614_A_T | 30590, 30593, | 30633, 30637, | 30666, 30671, |
| rs1491943_SNP | 30601, 30613, | 30639, 30650, | 30673-30674, |
| | 30616, 30698- | 30653, 30735- | 30688, 30762- |
| | 30734 | 30761 | 30794 |
| 12:40227006_C_G | 30795-30841 | 30842-30891 | 30892-30943 |
| rs1491942_REF | | | |
| 12:40227006_C_G | 30798, 30802, | 30845, 30848, | 30893, 30896, |
| rs1491942_SNP | 30804, 30815, | 30850, 30852, | 30899, 30903, |
| | 30823, 30825, | 30859, 30865, | 30910, 30916, |
| | 30827, 30829, | 30873, 30879, | 30924, 30930, |
| | 30831, 30838, | 30881, 30888, | 30932, 30935, |
| | 30944-30981 | 30982-31021 | 31022-31063 |
| 12:40227013_G_A | 30796-30797, | 30843-30844, | 30892-30895, |
| rs1491941_REF | 30800-30801, | 30846-30849, | 30897-30900, |
| | 30803-30814, | 30851-30858, | 30902-30909, |
| | 30816-30820, | 30860-30861, | 30911-30912, |
| | 30823-30825, | 30863-30864, | 30914-30915, |
| | 30828, 30830- | 30866-30870, | 30917-30921, |
| | 30835, 30838- | 30873-30875, | 30924-30926, |
| | 30840, 31064- | 30878, 30880- | 30929, 30931- |
| | 31077 | 30885, 30888- | 30934, 30936- |
| | | 30890, 31078- | 30937, 30940- |
| | | 31091 | 30942, 31092- |
| | | | 31105 |
| 12:40227013_G_A | 30801, 30806, | 30846, 30849, | 30892, 30897, |
| rs1491941_SNP | 30816, 30819, | 30854, 30866, | 30905, 30917, |
| | 30824, 31067, | 30869, 31081, | 30920, 31093, |
| | 31074-31077, | 31083, 31089- | 31095, 31097, |
| | 31106-31143 | 31091, 31144- | 31104-31105, |
| | | 31183 | 31184-31225 |
| 12:40228090_A_G | 31226-31247 | 31248-31269 | 31270-31291 |
| rs11175645_REF | | | |
| 12:40228090_A_G | 31226-31227, | 31248-31249, | 31271, 31274, |
| rs11175645_SNP | 31229, 31237- | 31252, 31259- | 31276, 31281- |
| | 31238, 31292- | 31260, 31308- | 31282, 31325- |
| | 31307 | 31324 | 31341 |
| 12:40228102_C_T | 31226-31232, | 31248-31256, | 31270-31291, |
| rs4567538_REF | 31234, 31236- | 31258-31269, | 31388-31410 |
| | 31247, 31342- | 31365-31387 | |
| | 31364 | | |
| 12:40228102_C_T | 31247, 31347, | 31370, 31374, | 31397, 31406, |
| rs4567538_SNP | 31351, 31360- | 31384, 31387, | 31409-31410, |
| | 31361, 31364, | 31441-31473 | 31474-31510 |
| | 31411-31440 | | |
| 12:40228988_C_T | 31511-31558 | 31559-31604 | 31605-31654 |
| rs12230685_REF | | | |
| 12:40228988_C_T | 31511-31513, | 31559-31562, | 31605-31608, |
| rs12230685_SNP | 31515, 31521, | 31569, 31574, | 31610, 31621, |
| | 31526, 31537, | 31585, 31599, | 31623, 31648, |
| | 31544, 31552- | 31689-31721 | 31722-31757 |
| | 31553, 31655- | | |
| | 31688 | | |
| 12:40229925_G_A | 31758-31804 | 31805-31854 | 31855-31906 |
| rs11175655_REF | | | |
| 12:40229925_G_A | 31763, 31767, | 31810, 31813, | 31860, 31863, |
| rs11175655_SNP | 31779, 31782, | 31827, 31830, | 31868, 31881, |
| | 31788, 31792, | 31836, 31847, | 31886, 31888, |
| | 31798, 31801, | 31850, 31852- | 31902, 31904- |
| | 31803-31804, | 31854, 31945- | 31906, 31985- |
| | 31907-31944 | 31984 | 32026 |
| 12:40229953_G_A | 32027-32048 | 32049-32070 | 32071-32092 |
| rs11175656_REF | | | |
| 12:40229953_G_A | 32032-32033, | 32054-32055, | 32076-32078, |
| rs11175656_SNP | 32036-32037, | 32059-32060, | 32082-32083, |
| | 32039, 32093- | 32062, 32110- | 32127-32143 |
| | 32109 | 32126 | |
| 12:40229955_CTTTTTTTTTTT | 32027-32031, | 32049-32053, | 32071-32075, |
| TT_C | 32033-32036, | 32055-32058, | 32077-32081, |
| rs1402299410_REF | 32038-32041, | 32060-32063, | 32083-32085, |
| | 32043, 32045- | 32065, 32067- | 32087, 32089- |
| | 32048 | 32070 | 32092 |
| 12:40229955_CTTTTTTTTTTT | 32027-32031, | 32049-32053, | 32071-32075, |
| C | 32033-32036, | 32055-32058, | 32077-32081, |
| rs780970090_REF | 32038-32041, | 32060-32063, | 32083-32085, |
| | 32043, 32045- | 32065, 32067- | 32087, 32089- |
| | 32048 | 32070 | 32092 |
| 12:40229955_CT_C | 32027-32031, | 32049-32053, | 32071-32075, |
| rs1479481931_REF | 32033-32036, | 32055-32058, | 32077-32081, |
| | 32038-32041, | 32060-32063, | 32083-32085, |
| | 32043, 32045- | 32065, 32067- | 32087, 32089- |
| | 32048 | 32070 | 32092 |
| 12:40229955_C_CT | 32027-32031, | 32049-32053, | 32071-32075, |
| rs71078229_REF | 32033-32036, | 32055-32058, | 32077-32081, |
| | 32038-32041, | 32060-32063, | 32083-32085, |
| | 32043, 32045- | 32065, 32067- | 32087, 32089- |
| | 32048 | 32070 | 32092 |
| 12:40230508_A_G | 32144-32190 | 32191-32238 | 32239-32290 |
| rs11175658_REF | | | |
| 12:40230508_A_G | 32144, 32148, | 32191, 32195, | 32239, 32243, |
| rs11175658_SNP | 32150, 32159, | 32197, 32204, | 32252, 32255, |
| | 32163, 32165, | 32207, 32211, | 32261, 32264, |
| | 32170, 32172, | 32215, 32219, | 32268, 32282- |
| | 32183, 32187, | 32233, 32237, | 32283, 32287, |
| | 32291-32328 | 32329-32368 | 32369-32410 |
| 12:40230668_CT_C | 32411-32422 | 32423-32430 | 32431-32440 |
| rs3057613_REF | | | |
| 12:40230668_C_CT | 32411-32422 | 32423-32430 | 32431-32440 |
| rs35642658_REF | | | |
| 12:40231279_G_A | 32441-32463 | 32464-32488 | 32489-32515 |
| rs74324737_REF | | | |
| 12:40231279_G_A | 32441-32442, | 32464, 32469, | 32494, 32503- |
| rs74324737_SNP | 32453, 32458, | 32478, 32483, | 32504, 32510, |
| | 32463, 32516- | 32488, 32540- | 32515, 32566- |
| | 32539 | 32565 | 32593 |
| 12:40231365_C_A | 32594-32598 | 32599-32605 | 32606-32616 |
| rs2708441_REF | | | |
| 12:40231365_C_A | 32594-32595, | 32599-32600, | 32606, 32609, |
| rs2708441_SNP | 32598, 32617- | 32604-32605, | 32613-32614, |
| | 32633 | 32634-32656 | 32616, 32657- |
| | | | 32682 |
| 12:40231390_C_CAAAA | 32683-32697 | 32698-32713 | 32610, 32616, |
| rs10631840_REF | | | 32714-32728 |
| 12:40231390_C_CAAA | 32683-32697 | 32698-32713 | 32610, 32616, |
| rs35991926_REF | | | 32714-32728 |
| 12:40231570_C_T | 32729-32733 | 32734-32737 | 32738-32742 |
| rs61931634_SNP | | | |
| 12:40234631_C_T | 32743-32764 | 32765-32784 | 32785-32812 |
| rs11175666_REF | | | |
| 12:40234631_C_T | 32743, 32753, | 32765, 32775, | 32785, 32797, |
| | 32755, 32761, | 32777, 32779, | 32801, 32808, |
| rs11175666_SNP | 32813-32834 | 32835-32858 | 32859-32885 |
| 12:40235793_GTTT_G | 32886-32888 | 32889-32890 | 32891-32893 |
| rs751672176_REF | | | |
| 12:40235793_G_GTT | 32886-32888 | 32889-32890 | 32891-32893 |
| rs36024911_REF | | | |
| 12:40235793_G_GT | 32886-32888 | 32889-32890 | 32891-32893 |
| rs36024911_REF | | | |
| 12:40235794_T_TG | 32886, 32888 | 32889 | 32892-32893 |
| rs377568728_REF | | | |
| 12:40235794_T_TG | 32888 | , | 32892 |
| rs377568728_SNP | | | |
| 12:40235813_G_T | 32894-32909 | 32910-32925 | 32926-32943 |
| rs1279923177_REF | | | |
| 12:40235813_G_T | 32894, 32896- | 32910, 32912- | 32926-32927, |
| rs1279923177_SNP | 32897, 32902, | 32914, 32920, | 32930-32931, |
| | 32904, 32944- | 32953-32961 | 32937, 32962- |
| | 32952 | | 32972 |
| 12:40237556_T_C | 32973-33020 | 33021-33070 | 33071-33122 |
| rs2249281_REF | | | |
| 12:40237556_T_C | 32980, 32987, | 33028, 33035, | 33078, 33087- |
| rs2249281_SNP | 32989, 32997, | 33037, 33040, | 33088, 33091, |
| | 33006, 33008, | 33046, 33055, | 33093, 33098, |
| | 33011-33013, | 33060-33062, | 33107, 33112- |
| | 33016, 33123- | 33066, 33161- | 33113, 33118, |
| | 33160 | 33200 | 33201-33242 |
| 12:40237806_G_A | 33243-33286 | 33287-33331 | 33332-33378 |
| rs10878244_REF | | | |
| 12:40237806_G_A | 33252, 33264, | 33296, 33308- | 33341-33342, |
| rs10878244_SNP | 33268, 33272, | 33309, 33313, | 33354-33355, |
| | 33282, 33286, | 33331, 33416- | 33359, 33451- |
| | 33379-33415 | 33450 | 33491 |
| 12:40237823_CAACA_C | 33253, 33257, | 33297, 33301- | 33337, 33343, |
| rs138000936_REF | 33261, 33269- | 33302, 33305, | 33347-33348, |
| | 33270, 33272, | 33314-33315, | 33351, 33360- |
| | 33278, 33280- | 33317, 33323, | 33361, 33363, |
| | 33281, 33285, | 33325-33326, | 33369, 33371- |
| | 33492-33494 | 33330, 33495 | 33372, 33375, |
| | | | 33377, 33496 |
| 12:40237823_CAACA_C | 33497 | , | |
| rs138000936_SNP | | | |
| 12:40237989_T_C | 33498-33540 | 33541-33576 | 33577-33617 |
| rs10878245_REF | | | |
| 12:40237989_T_C | 33499, 33501, | 33543, 33547- | 33578, 33580, |
| rs10878245_SNP | 33506-33507, | 33548, 33563, | 33585-33586, |
| | 33526-33527, | 33565, 33656- | 33600, 33606, |
| | 33529, 33618- | 33695 | 33696-33737 |
| | 33655 | | |
| 12:40238297_T_G | 33738-33785 | 33786-33829 | 33830-33877 |
| rs10878246_REF | | | |
| 12:40238297_T_G | 33742, 33749, | 33789, 33794, | 33833, 33838, |
| rs10878246_SNP | 33759-33761, | 33798, 33801, | 33842, 33845, |
| | 33768, 33776, | 33807-33808, | 33850, 33853, |
| | 33781, 33784- | 33815, 33823, | 33860, 33864, |
| | 33785, 33878- | 33827, 33829, | 33869, 33874, |
| | 33915 | 33916-33955 | 33956-33997 |
| 12:40238298_C_T | 33738-33754, | 33786-33800, | 33830-33845, |
| rs10878247_REF | 33756-33780, | 33803-33829, | 33847-33849, |
| | 33782-33785, | 34000-34001 | 33851-33877, |
| | 33998-33999 | | 34002-34003 |
| 12:40238298_C_T | 33742, 33749, | 33789, 33794, | 33833, 33838, |
| rs10878247_SNP | 33760-33761, | 33798, 33808, | 33842, 33845, |
| | 33768, 33776, | 33815, 33823, | 33860, 33864, |
| | 33780, 33784- | 33827-33829, | 33869, 33874, |
| | 33785, 33998, | 34000, 34032- | 33877, 34002, |
| | 34004-34031 | 34053 | 34054-34081 |
| 12:40239156_G_T | 34082-34129 | 34130-34179 | 34180-34231 |
| rs954884_REF | | | |
| 12:40239156_G_T | 34092, 34103, | 34138, 34141, | 34188, 34191, |
| rs954884_SNP | 34105, 34107, | 34152, 34154, | 34202, 34206, |
| | 34111, 34116, | 34156, 34160, | 34210-34211, |
| | 34121, 34123, | 34169, 34171, | 34215, 34221, |
| | 34126, 34129, | 34176, 34179, | 34223, 34231, |
| | 34232-34269 | 34270-34309 | 34310-34351 |
| 12:40239512_T_C | 34352-34399 | 34400-34449 | 34450-34501 |
| rs10878249_REF | | | |
| 12:40239512_T_C | 34352, 34355, | 34400, 34418, | 34458, 34469, |
| rs10878249_SNP | 34370, 34378, | 34426, 34429, | 34477, 34480, |
| | 34381-34383, | 34431, 34438, | 34482, 34489, |
| | 34394-34395, | 34443-34444, | 34494, 34498- |
| | 34398, 34502- | 34447-34448, | 34499, 34501, |
| | 34539 | 34540-34579 | 34580-34621 |
| 12:40239686_G_A | 34622-34652 | 34653-34682 | 34683-34716 |
| rs10784444_REF | | | |
| 12:40239686_G_A | 34634, 34641, | 34657, 34664, | 34688, 34695, |
| rs10784444_SNP | 34647, 34650, | 34671, 34677, | 34698, 34711, |
| | 34717-34739 | 34680, 34740- | 34714, 34757- |
| | | 34756 | 34775 |
| 12:40240356_T_C | 34776-34787 | 34788-34799 | 34800-34813 |
| rs6581622_REF | | | |
| 12:40240356_T_C | 34776, 34778- | 34788, 34790, | 34803, 34806, |
| rs6581622_SNP | 34779, 34782, | 34793, 34795, | 34808, 34811, |
| | 34784, 34814- | 34798, 34825- | 34813, 34834- |
| | 34824 | 34833 | 34844 |
| 12:40241554_G_A | 34845-34882 | 34883-34914 | 34915-34952 |
| rs17519573_REF | | | |
| 12:40241554_G_A | 34853, 34863, | 34888, 34896, | 34922-34923, |
| rs17519573_SNP | 34868, 34872, | 34903, 34909, | 34932, 34940, |
| | 34880, 34953- | 34912, 34976- | 34946, 34995- |
| | 34975 | 34994 | 35015 |
| 12:40242804_C_CAAAAAAA | 35016-35028 | 35029-35041 | 35042-35056 |
| AAAAAAAAAA | | | |
| rs35987733_REF | | | |
| 12:40242804_C_CAAAAAA | 35016-35028 | 35029-35041 | 35042-35056 |
| rs35987733_REF | | | |
| 12:40242818_G_A | 35057-35062 | 35063-35068 | 35069-35076 |
| rs868476079_SNP | | | |
| 12:40242819_G_A | 35057, 35060- | 35063-35064, | 35070-35072, |
| rs1399756142_SNP | 35062, 35077- | 35067-35068, | 35075, 35085- |
| | 35080 | 35081-35084 | 35090 |
| 12:40242820_T_A_._SNP | 35057, 35061- | 35063-35064, | 35070, 35072, |
| | 35062, 35077, | 35067, 35082, | 35075, 35086, |
| | 35091-35100 | 35101-35107 | 35108-35116 |
| 12:40242823_G_A | 35077, 35096, | 35082, 35131- | 35086, 35135- |
| rs1474763022_SNP | 35117-35130 | 35134 | 35141 |
| 12:40242824_C_A | 35096, 35119, | 35155-35158 | 35159-35167 |
| rs1450276224_SNP | 35127, 35130, | | |
| | 35142-35154 | | |
| 12:40242827_T_A | 35130, 35145, | 35194-35212 | 35160, 35213- |
| rs1191097078_SNP | 35168-35193 | | 35233 |
| 12:40242829_T_A | 35179-35180, | 35200-35201, | 35221, 35229, |
| rs_1425272990_SNP | 35191, 35234- | 35211, 35263- | 35232, 35281- |
| | 35262 | 35280 | 35301 |
| 12:40242832_C_A | 35254, 35260, | 35266, 35277, | 35283, 35287, |
| rs1477924701_SNP | 35302-35319 | 35320-35329 | 35330-35345 |
| 12:40245759_A_G | 35346-35350 | , | 35351 |
| rs2404580_REF | | | |
| 12:40245759_A_G | 35348, 35352- | 35377-35382 | 35351, 35383- |
| rs2404580_SNP | 35376 | | 35392 |
| 12:40246099_C_T | 35393-35404 | 35405-35412 | 35413-35422 |
| rs2404581_REF | | | |
| 12:40246099_C_T | 35395, 35398, | 35405, 35407- | 35414-35415, |
| rs2404581_SNP | 35401-35402, | 35408, 35411- | 35417-35418, |
| | 35404, 35423 | 35412, 35424 | 35421, 35425- |
| | | | 35427 |
| 12:40246358_T_C | , | , | 35428 |
| rs1388599_REF | | | |
| 12:40246358_T_C | 35429-35438 | , | 35428 |
| rs1388599_SNP | | | |
| 12:40246824_A_G | 35439-35485 | 35486-35535 | 35536-35587 |
| rs1388596_REF | | | |
| 12:40246824_A_G | 35446, 35452, | 35490, 35494, | 35540, 35550, |
| rs1388596_SNP | 35456, 35459, | 35504, 35507, | 35558-35559, |
| | 35463, 35466- | 35512, 35515- | 35564, 35567- |
| | 35467, 35472- | 35516, 35521, | 35568, 35573, |
| | 35473, 35479, | 35528, 35530, | 35580, 35582, |
| | 35588-35625 | 35626-35665 | 35666-35707 |
| 12:40247890_A_G | 35708-35747 | 35748-35785 | 35786-35825 |
| rs10878258_REF | | | |
| 12:40247890_A_G | 35715, 35718, | 35749, 35756, | 35786, 35788, |
| rs10878258_SNP | 35735, 35742, | 35759, 35775, | 35795, 35798, |
| | 35745, 35826- | 35783, 35864- | 35814, 35901- |
| | 35863 | 35900 | 35939 |
| 12:40250503_T_C | 35940-35943 | 35944-35946 | 35947-35950 |
| rs12371502_REF | | | |
| 12:40250503_T_C | 35940-35943 | 35944-35946 | 35947-35951 |
| rs12371502_SNP | | | |
| 12:40250565_C_T | 35952-35980 | 35981-36014 | 36015-36054 |
| rs11175729_REF | | | |
| 12:40250565_C_T | 35956-35957, | 35986, 35992, | 36022, 36024, |
| rs11175729_SNP | 35962, 35968, | 35998, 36002, | 36036, 36041- |
| | 35971, 35975, | 36006, 36008, | 36042, 36046, |
| | 36055-36075 | 36010, 36076- | 36048, 36050, |
| | | 36099 | 36100-36126 |
| 12:40250950_A_G | 36127-36139 | 36140-36156 | 36157-36174 |
| rs17443414_REF | | | |
| 12:40250950_A_G | 36127-36128, | 36140-36142, | 36157-36159, |
| rs17443414_SNP | 36130, 36132, | 36144, 36149, | 36161, 36166, |
| | 36135, 36175- | 36204-36236 | 36237-36273 |
| | 36203 | | |
| 12:40251002_G_GT | 36274-36291 | 36292-36309 | 36310-36327 |
| rs1486599371_REF | | | |
| 12:40251072_C_T | 36328-36349 | 36350-36367 | 36368-36387 |
| rs7134379_REF | | | |
| 12:40251072_C_T | 36330, 36332, | 36353, 36356, | 36371, 36375- |
| rs7134379_SNP | 36336, 36339, | 36358, 36363, | 36376, 36378, |
| | 36345, 36388- | 36365, 36399- | 36385, 36410- |
| | 36398 | 36409 | 36422 |
| 12:40251455_C_A | 36423-36467 | 36468-36511 | 36512-36559 |
| rs7955902_REF | | | |
| 12:40251455_C_A | 36425, 36431- | 36470, 36474, | 36514, 36520, |
| rs7955902_SNP | 36433, 36443, | 36476-36477, | 36522-36523, |
| | 36446, 36457, | 36490, 36501, | 36537, 36554, |
| | 36560-36593 | 36594-36625 | 36626-36661 |
| 12:40251794_A_G | 36662-36702 | 36703-36733 | 36734-36772 |
| rs1491939_REF | | | |
| 12:40251794_A_G | 36664, 36669, | 36706, 36719, | 36736, 36741, |
| rs1491939_SNP | 36677, 36684, | 36721, 36811- | 36755, 36757, |
| | 36686, 36688, | 36848 | 36759, 36849- |
| | 36773-36810 | | 36890 |
| 12:40251828_G_A | 36891-36925 | 36926-36959 | 36960-36995 |
| rs1491938_REF | | | |
| 12:40251828_G_A | 36894, 36896, | 36929, 36931, | 36963, 36965- |
| rs1491938_SNP | 36903, 36914, | 36939-36940, | 36966, 36975, |
| | 36922, 36996- | 36949, 37023- | 36985, 37038- |
| | 37022 | 37037 | 37056 |
| 12:40253146_G_A | 37057-37101 | 37102-37138 | 37139-37183 |
| rs7969677_REF | | | |
| 12:40253146_G_A | 37060, 37067, | 37109, 37117, | 37142, 37149, |
| rs7969677_SNP | 37073, 37075, | 37132 | 37173, 37205- |
| | 37091, 37099, | | 37211 |
| | 37101, 37184- | | |
| | 37204 | | |
| 12:40255521_C_T | 37212-37256 | 37257-37301 | 37302-37349 |
| rs2723265_REF | | | |
| 12:40255521_C_T | 37220, 37231- | 37267, 37277- | 37311, 37313, |
| rs2723265_SNP | 37232, 37237, | 37278, 37283, | 37323-37324, |
| | 37242, 37244, | 37289, 37291, | 37329, 37336, |
| | 37246, 37350- | 37383-37407 | 37339, 37408- |
| | 37382 | | 37440 |
| 12:40255947_G_C | 37441-37477 | 37478-37496 | 37497-37522 |
| rs7132171_REF | | | |
| 12:40255947_G_C | 37446, 37458- | 37482, 37490, | 37501, 37509, |
| rs7132171_SNP | 37459, 37466, | 37556-37572 | 37513, 37573- |
| | 37523-37555 | | 37595 |
| 12:40256086_G_A | 37596-37626 | 37627-37655 | 37656-37687 |
| rs10784451_REF | | | |
| 12:40256086_G_A | 37601, 37608- | 37629, 37638- | 37657, 37660, |
| | | 37639, 37649, | 37670-37671, |
| rs10784451_SNP | 37609, 37617, | 37711-37730 | 37681, 37731- |
| | 37620, 37688- | | 37752 |
| | 37710 | | |
| 12:40256173_G_A | 37753-37771 | 37772-37794 | 37795-37821 |
| rs10784452_REF | | | |
| 12:40256173_G__A | 37753, 37755- | 37773, 37776, | 37799, 37805- |
| rs10784452_SNP | 37756, 37762, | 37782, 37793- | 37806, 37819, |
| | 37771, 37822- | 37794, 37846- | 37821, 37874- |
| | 37845 | 37873 | 37903 |
| 12:40256549_C_T | 37904-37935 | 37936-37955 | 37956-37983 |
| rs1907633_REF | | | |
| 12:40256549_C_T | 37907, 37912, | 37936-37937, | 37956-37957, |
| rs1907633_SNP | 37915-37916, | 37939, 37941, | 37960, 37963, |
| | 37919, 37922, | 37943, 37946, | 37969, 37971, |
| | 37924, 37928, | 37948, 37950, | 37974, 37976, |
| | 37933-37934, | 37953-37954, | 37981-37982, |
| | 37984-37988 | 37989-37993 | 37994-38001 |
| 12:40256647_T_C | 38002-38008 | 38009-38010 | 38011-38013 |
| rs1491936_REF | | | |
| 12:40256647_T_C | 38002-38004, | 38009-38010, | 38011-38013, |
| rs1491936_SNP | 38007-38008, | 38035-38039 | 38040-38048 |
| | 38014-38034 | | |
| 12:40256700_G_A | 38049-38096 | 38097-38146 | 38147-38198 |
| rs1907632_REF | | | |
| 12:40256700_G_A | 38050, 38053, | 38097, 38099, | 38147, 38149, |
| rs1907632_SNP | 38059, 38067, | 38102, 38105, | 38152, 38155, |
| | 38072, 38076, | 38109, 38117, | 38159, 38163, |
| | 38079-38081, | 38129-38131, | 38168, 38182, |
| | 38090, 38199- | 38140, 38237- | 38185, 38192, |
| | 38236 | 38276 | 38277-38318 |
| 12:40258592_A_G | 38319-38366 | 38367-38416 | 38417-38468 |
| rs2046928_REF | | | |
| 12:40258592_A_G | 38324, 38326, | 38372, 38374- | 38424-38425, |
| rs2046928_SNP | 38342-38344, | 38375, 38387, | 38432, 38435, |
| | 38348, 38354, | 38392-38393, | 38439, 38444- |
| | 38360-38361, | 38398, 38404, | 38445, 38450, |
| | 38365, 38469- | 38410-38411, | 38462-38463, |
| | 38506 | 38507-38546 | 38547-38588 |
| 12:40258718_T_C | 38589-38636 | 38637-38686 | 38687-38738 |
| rs2723264_REF | | | |
| 12:40258718_T_C | 38591, 38599- | 38639, 38641, | 38691, 38699, |
| rs2723264_SNP | 38600, 38605- | 38649, 38654- | 38704-38706, |
| | 38607, 38609, | 38656, 38658, | 38708, 38710, |
| | 38611, 38618, | 38660, 38667, | 38714, 38719, |
| | 38631, 38739- | 38680, 38777- | 38732, 38817- |
| | 38776 | 38816 | 38858 |
| 12:40259911_C_T | 38859-38896 | 38897-38932 | 38933-38969 |
| rs17465870_REF | | | |
| 12:40259911_C_T | 38876, 38884, | 38926, 39005- | 38950, 38966, |
| | 38889, 38893, | | |
| rs17465870_SNP | 38970-39004 | 39039 | 39040-39074 |
| 12:40260186_C_T | 39075-39121 | 39122-39164 | 39165-39209 |
| rs10878278_REF | | | |
| 12:40260186_C_T | 39075, 39078- | 39122, 39125, | 39193, 39196, |
| | 39079, 39085, | 39150, 39248- | |
| | 39089, 39104, | | |
| rs10878278_SNP | 39107, 39115, | 39283 | 39204, 39284- |
| | 39117, 39210- | | 39325 |
| | 39247 | | |
| 12:40261214_T_C | 39326-39329 | , | , |
| rs11175766_REF | | | |
| 12:40261214_T_C | 39326-39335 | , | 39336 |
| rs11175766_SNP | | | |
| 12:40261905_C_T | 39337-39350 | 39351-39354 | 39355-39360 |
| rs11564183_REF | | | |
| 12:40262377_A_G | 39361-39398 | 39399-39435 | 39436-39475 |
| rs11175769_REF | | | |
| 12:40262377_A_G | 39373, 39382, | 39411, 39419, | 39449, 39453, |
| rs11175769_SNP | 39386, 39388, | 39425, 39428, | 39458, 39464, |
| | 39391, 39395, | 39433-39434, | 39467, 39473, |
| | 39397, 39476- | 39509-39541 | 39475, 39542- |
| | 39508 | | 39577 |
| 12:40262593_C_T | 39578-39619 | 39620-39662 | 39663-39706 |
| rs17465912_REF | | | |
| 12:40262593_C_T | 39587, 39601, | 39644, 39652, | 39686, 39688, |
| rs17465912_SNP | 39609, 39614, | 39657, 39660, | 39700, 39703, |
| | 39617, 39707- | 39744-39778 | 39706, 39779- |
| | 39743 | | 39815 |
| 12:40262875_C_A | 39816-39863 | 39864-39913 | 39914-39965 |
| rs17443552_REF | | | |
| 12:40262875_C_A | 39817-39818, | 39865-39866, | 39915, 39920, |
| rs17443552_SNP | 39824, 39828- | 39872, 39876, | 39923, 39927, |
| | 39829, 39837, | 39885, 39888- | 39936, 39939- |
| | 39840, 39850, | 39889, 39899, | 39940, 39953, |
| | 39854, 39863, | 39902, 39904, | 39955, 39962, |
| | 39966-40003 | 40004-40043 | 40044-40085 |
| 12:40263160_T_A | 40086-40088 | | , |
| rs17490817_REF | | | |
| 12:40263160_T_A | 40086-40088 | | , |
| rs17490817_SNP | | | |
| 12:40263401_G_GA | 40089-40120 | 40121-40142 | 40143-40170 |
| rs11430638_REF | | | |
| 12:40263401_G_GA | 40094, 40104, | 40122, 40129, | 40146, 40154, |
| rs11430638_SNP | 40115, 40119, | 40134, 40141, | 40160, 40166, |
| | 40171-40192 | 40193-40204 | 40205-40222 |
| 12:40263423_G_A | 40094, 40103, | 40122, 40128, | 40146, 40153, |
| rs4293189_REF | 40110, 40114, | 40134-40136, | 40159-40160, |
| | 40223-40265 | 40139, 40266- | 40162-40163, |
| | | 40309 | 40166-40167, |
| | | | 40310-40353 |
| 12:40263423_G_A | 40103, 40114, | 40128, 40135- | 40153, 40162- |
| rs4293189_SNP | 40225, 40227, | 40136, 40268, | 40163, 40167, |
| | 40233, 40235, | 40271-40272, | 40312, 40315- |
| | 40238, 40240, | 40280, 40283, | 40316, 40324, |
| | 40242, 40246, | 40285, 40291, | 40333, 40336, |
| | 40354-40391 | 40392-40425 | 40426-40463 |
| 12:40263735_A_G | 40464-40495 | 40496-40524 | 40525-40555 |
| rs10784461_REF | | | |
| 12:40263735_A_G | 40478, 40487- | 40510, 40518, | 40536, 40549, |
| rs10784461_SNP | 40488, 40491, | 40520, 40523, | 40551, 40554, |
| | 40494, 40556- | 40591-40619 | 40620-40652 |
| | 40590 | | |
| 12:40263898_C_G | 6533, 6628, | 6560, 6577, | 6599, 6603, |
| rs7308720_REF | 6631, 6644, | 6663, 6665, | 6622, 6691, |
| | 6651, 6659, | 6668, 6678, | 6693, 6696, |
| | 6727, 6737- | 6689, 6762, | 6701, 6708, |
| | 6738, 6742, | 6771-6772, | 6804-6805, |
| | 6747, 40653- | 6779, 6783, | 6814-6815, |
| | 40685 | 40686-40716 | 6819, 40717- |
| | | | 40750 |
| 12:40263898_C_G | 6628, 6631, | 6663, 6665, | 6691, 6693, |
| rs7308720_SNP | 6644, 40653, | 6668, 40687, | 6701, 40720, |
| | 40656, 40660, | 40696, 40789- | 40731, 40827- |
| | 40665, 40751- | 40826 | 40868 |
| | 40788 | | |
| 12:40263969_C_G | 40869-40913 | 40914-40956 | 40957-41003 |
| rs10784462_REF | | | |
| 12:40263969_C_G | 40870, 40879, | 40931, 40938, | 40970, 40975, |
| rs10784462_SNP | 40887, 40889, | 40953, 41042- | 40977, 40984, |
| | 40895, 40905, | 41081 | 40997, 41082- |
| | 40910, 41004- | | 41123 |
| | 41041 | | |
| 12:40264403_G_A | 41124-41127 | 41128-41133 | 41134-41139 |
| rs10878284_REF | | | |
| 12:40265294_C_T | 41140-41187 | 41188-41237 | 41238-41289 |
| rs11175780_REF | | | |
| 12:40265294_C_T | 41157, 41168, | 41217, 41219, | 41247, 41250, |
| rs11175780_SNP | 41170, 41174, | 41223-41224, | 41269, 41271, |
| | 41177, 41182- | 41227, 41232- | 41275-41276, |
| | 41183, 41185- | 41233, 41235- | 41285, 41287- |
| | 41187, 41290- | 41237, 41328- | 41289, 41368- |
| | 41327 | 41367 | 41409 |
| 12:40265732_C_T | 41410-41450 | 41451-41493 | 41494-41537 |
| rs11175784_REF | | | |
| 12:40265732_C_T | 41411, 41414, | 41451, 41453, | 41494, 41496, |
| rs11175784_SNP | 41421, 41424, | 41456, 41463, | 41499, 41506, |
| | 41442, 41538- | 41485, 41570- | 41516, 41604- |
| | 41569 | 41603 | 41640 |
| 12:40266292_C_A | 41641 | , | 41642 |
| rs11175787_REF | | | |
| 12:40267078_A_T | , | 41643 | 41644-41646 |
| rs144463374_SNP | | | |
| 12:40267251_A_T | 41647-41691 | 41692-41738 | 41739-41787 |
| rs73108329_REF | | | |
| 12:40267251_A_T | 41655-41656, | 41701, 41708, | 41742, 41744, |
| rs73108329_SNP | 41663, 41668, | 41710, 41714, | 41750, 41757, |
| | 41679, 41682, | 41726, 41729, | 41759, 41763, |
| | 41685, 41688, | 41735, 41826- | 41784, 41866- |
| | 41788-41825 | 41865 | 41907 |
| 12:40267479_T_C | 41908-41953 | 41954-42003 | 42004-42055 |
| rs12426891_REF | | | |
| 12:40267479_T_C | 41908, 41914, | 41962, 41966- | 42012, 42016- |
| rs12426891_SNP | 41916, 41920- | 41967, 41973, | 42017, 42026, |
| | 41921, 41927, | 41976, 41984- | 42035, 42041- |
| | 41930, 41938- | 41985, 41991, | 42042, 42047- |
| | 41939, 41948, | 41996-41997, | 42048, 42054, |
| | 42056-42093 | 42094-42133 | 42134-42175 |
| 12:40267601_A_T | 42176-42223 | 42224-42273 | 42274-42325 |
| rs4768224_REF | | | |
| 12:40267601_A_T | 42184, 42191, | 42232, 42235, | 42282-42283, |
| rs4768224_SNP | 42201-42205, | 42240, 42251, | 42286, 42291, |
| | 42214-42216, | 42253-42255, | 42302-42303, |
| | 42326-42363 | 42264-42266, | 42306-42307, |
| | | 42364-42403 | 42317-42318, |
| | | | 42404-42445 |
| 12:40268237_G_A | 42446-42493 | 42494-42543 | 42544-42595 |
| rs11175793_REF | | | |
| 12:40268237_G_A | 42450, 42459, | 42498, 42507, | 42548, 42557, |
| rs11175793_SNP | 42463-42464, | 42511, 42514, | 42564-42565, |
| | 42466, 42469, | 42521, 42524, | 42572, 42575, |
| | 42473, 42476, | 42532, 42534, | 42583, 42587, |
| | 42485, 42487, | 42536, 42541, | 42592, 42594, |
| | 42596-42633 | 42634-42673 | 42674-42715 |
| 12:40269794_G_T | 42716-42763 | 42764-42813 | 42814-42865 |
| rs10784470_REF | | | |
| 12:40269794_G_T | 42717, 42721- | 42764, 42766, | 42814-42815, |
| rs10784470_SNP | 42722, 42728, | 42770-42771, | 42817, 42821- |
| | 42731, 42738- | 42777, 42780- | 42822, 42828, |
| | 42739, 42741, | 42781, 42788, | 42831-42832, |
| | 42743, 42755, | 42793, 42805, | 42844, 42850, |
| | 42866-42901 | 42902-42923 | 42924-42957 |
| 12:40270116_C_G | 42958-42993 | 42994-43015 | 43016-43041 |
| rs12146857_REF | | | |
| 12:40270116_C_G | 42964, 42968, | 42996, 42998, | 43019, 43022, |
| rs12146857_SNP | 42976, 42984, | 43001, 43003, | 43025, 43029, |
| | 42987, 43042- | 43010, 43073- | 43035, 43090- |
| | 43072 | 43089 | 43110 |
| 12:40270841_T_A | 43111-43131 | 43132-43145 | 43146-43163 |
| rs11564207_REF | | | |
| 12:40270841_T_A | 43119, 43131, | 43183-43196 | 43197-43214 |
| rs11564207_SNP | 43164-43182 | | |
| 12:40271425_G_C | 43215-43262 | 43263-43309 | 43310-43358 |
| rs7308193_REF | | | |
| 12:40271425_G_C | 43222, 43225, | 43272, 43295, | 43319, 43321, |
| rs7308193_SNP | 43231, 43246, | 43302, 43304, | 43341, 43351, |
| | 43249, 43251, | 43306, 43308- | 43355, 43357- |
| | 43256, 43258, | 43309, 43397- | 43358, 43437- |
| | 43260, 43262, | 43436 | 43478 |
| | 43359-43396 | | |
| 12:40272958_A_T | 43479-43524 | 43525-43566 | 43567-43616 |
| rs10506150_REF | | | |
| 12:40272958_A_T | 43495, 43498- | 43539, 43542- | 43579, 43584, |
| rs10506150_SNP | 43499, 43501, | 43543, 43545, | 43587-43588, |
| | 43509, 43512, | 43552-43553, | 43593, 43599- |
| | 43517-43519, | 43556, 43561, | 43600, 43603, |
| | 43524, 43617- | 43565-43566, | 43610, 43615, |
| | 43652 | 43653-43684 | 43685-43724 |
| 12:40273002_GAC_G | 43725-43772 | 43773-43822 | 43823-43874 |
| rs76660489_REF | | | |
| 12:40273002_GAC_G | 43727, 43732, | 43775, 43781, | 43825, 43827, |
| rs76660489_SNP | 43741, 43749- | 43789, 43794, | 43832, 43845, |
| | 43750, 43759, | 43798-43799, | 43849-43850, |
| | 43769, 43772, | 43809, 43822, | 43853, 43860, |
| | 43875-43913 | 43914-43954 | 43955-43997 |
| 12:40273073_C_G | 43998-44028 | 44029-44044 | 44045-44065 |
| rs10878299_REF | | | |
| 12:40273073_C_G | 44007, 44011, | 44035, 44093- | 44055, 44061, |
| rs10878299_SNP | 44014, 44023, | 44107 | 44108-44126 |
| | 44066-44092 | | |
| 12:40275500_TTAAGAA_T | 44127-44155 | 44156-44183 | 44184-44215 |
| rs373619252_REF | | | |
| 12:40275500_TTAAGAA_T | 44130-44131, | 44157-44158, | 44186, 44193, |
| rs373619252_SNP | 44139, 44142, | 44164, 44166, | 44195, 44198, |
| | 44145, 44216- | 44172, 44236- | 44204, 44254- |
| | 44235 | 44253 | 44275 |
| 12:40276024_T_A | 44276-44277 | , | , |
| rs10878302_REF | | | |
| 12:40276024_T_A | 44276-44277 | , | , |
| rs10878302_SNP | | | |
| 12:40276137_T_C | 44278-44300 | 44301-44308 | 44309-44323 |
| rs11564129_REF | | | |
| 12:40276137_T_C | 44283, 44288- | 44301, 44303, | 44310, 44313- |
| rs11564129_SNP | 44289, 44291- | 44306-44308, | 44314, 44318, |
| | 44292, 44295, | 44353-44373 | 44320-44321, |
| | 44324-44352 | | 44374-44397 |
| 12:40276592_G_A | 44398-44427 | 44428-44460 | 44461-44495 |
| rs7971935_REF | | | |
| 12:40276592_G_A | 44405, 44417, | 44449, 44459- | 44469, 44483, |
| rs7971935_SNP | 44425-44427, | 44460, 44516- | 44495, 44538- |
| | 44496-44515 | 44537 | 44561 |
| 12:40276874_C_T | 44562-44570 | 44571-44583 | 44584-44600 |
| rs11175836_REF | | | |
| 12:40276874_C_T | 44563, 44566, | 44571, 44576, | 44584, 44590, |
| rs11175836_SNP | 44601-44628 | 44581, 44629- | 44596, 44599, |
| | | 44661 | 44662-44695 |
| 12:40276999_C_T | 44696-44731 | 44732-44763 | 44764-44798 |
| rs4272849_REF | | | |
| 12:40276999_C_T | 44713, 44717, | 44738, 44747, | 44770, 44783, |
| rs4272849_SNP | 44728, 44799- | 44750, 44825- | 44792, 44851- |
| | 44824 | 44850 | 44878 |
| 12:40277068_T_C | 44879-44889 | 44890-44903 | 44904-44923 |
| rs10784485_REF | | | |
| 12:40277068_T_C | 44879-44880, | 44891-44892, | 44906, 44908- |
| rs10784485_SNP | 44882, 44885- | 44894-44896, | 44910, 44916, |
| | 44886, 44924- | 44900, 44945- | 44918-44919, |
| | 44944 | 44968 | 44969-44995 |
| 12:40277196_C_A | 44996-45033 | 45034-45063 | 45064-45099 |
| rs10506151_REF | | | |
| 12:40277196_C_A | 44996, 44998, | 45034-45035, | 45064-45065, |
| rs10506151_SNP | 45002, 45010, | 45038, 45044, | 45077, 45085, |
| | 45023, 45100- | 45051, 45123- | 45094, 45146- |
| | 45122 | 45145 | 45170 |
| 12:40279458_T_A | 45171-45206 | 45207-45237 | 45238-45274 |
| rs7309197_REF | | | |
| 12:40279458_T_A | 45177, 45185, | 45220, 45235, | 45245, 45253, |
| rs7309197_SNP | 45191, 45200, | 45306-45334 | 45335-45369 |
| | 45204, 45275- | | |
| | 45305 | | |
| 12:40279566_G_A | 45370-45395 | 45396-45403 | 45404-45417 |
| rs7134572_REF | | | |
| 12:40279566_G_A | 45373, 45382- | 45396, 45398, | 45404, 45407, |
| rs7134572_SNP | 45384, 45418- | 45401-45402 | 45411, 45414- |
| | 45419 | | 45415 |
| 12:40280237_G_C | 45420-45422 | 45423-45427 | 45428-45434 |
| rs11175843_REF | | | |
| 12:40280237_G_C | 45421-45422, | 45424, 45426- | 45429, 45431- |
| rs11175843_SNP | 45435-45446 | 45427, 45447- | 45432, 45434, |
| | | 45461 | 45462-45483 |
| 12:40280621_T_TTAAAATAA | 45484-45504 | 45505-45527 | 45528-45554 |
| AA | | | |
| rs147801391_REF | | | |
| 12:40280621_T_TTAAAA | 45484-45504 | 45505-45527 | 45528-45554 |
| rs147801391_REF | | | |
| 12:40280621_T_TTAAATAAA | 45484-45504 | 45505-45527 | 45528-45554 |
| ATAAAA | | | |
| rs147801391_REF | | | |
| 12:40280621_T_TTAAATAAA | 45484, 45490, | 45505, 45511, | 45528, 45533, |
| ATAAAA | 45495, 45555- | 45517, 45519, | 45536, 45542, |
| rs147801391_SNP | 45568 | 45569-45581 | 45545, 45582- |
| | | | 45595 |
| 12:40280621_T_TTAAATAAA | 45484-45504 | 45505-45527 | 45528-45554 |
| A | | | |
| rs147801391_REF | | | |
| 12:40280621_T_TTAAATAAA | 45484, 45490, | 45505, 45511, | 45528, 45533, |
| A | 45495, 45555- | 45517, 45519, | 45536, 45542, |
| rs147801391_SNP | 45568 | 45569-45581 | 45545, 45582- |
| | | | 45595 |
| 12:40280622_TA_T | 45484, 45486- | 45505, 45507- | 45528, 45530- |
| rs376096297_REF | 45504 | 45527 | 45554 |
| 12:40280622_TA_T | 45484, 45490, | 45505, 45511, | 45528, 45533, |
| rs376096297_SNP | 45495, 45555- | 45517, 45519, | 45536, 45542, |
| | 45568 | 45569-45581 | 45545, 45582- |
| | | | 45595 |
| 12:40280792_T_C | 45596-45622 | 45623-45651 | 45652-45682 |
| rs7488873_REF | | | |
| 12:40280792_T_C | 45612-45613, | 45640, 45645, | 45669-45670, |
| rs7488873_SNP | 45617, 45620, | 45648-45649, | 45676, 45680, |
| | 45622, 45683- | 45651, 45704- | 45682, 45719- |
| | 45703 | 45718 | 45738 |
| 12:40281250_A_G | 45739-45786 | 45787-45834 | 45835-45886 |
| rs11175845_REF | | | |
| 12:40281250_A_G | 45745, 45753, | 45792, 45800, | 45841-45842, |
| rs11175845_SNP | 45755, 45758, | 45802, 45805, | 45850, 45852, |
| | 45761, 45765, | 45808, 45812, | 45855, 45862, |
| | 45768, 45772, | 45815-45816, | 45866-45867, |
| | 45775, 45780, | 45824, 45829, | 45875, 45880, |
| | 45887-45924 | 45925-45964 | 45965-46006 |
| 12:40281806_A_G | 46007-46052 | 46053-46096 | 46097-46144 |
| rs7979341_REF | | | |
| 12:40281806_A_G | 46017, 46019, | 46056, 46065, | 46098, 46101, |
| rs7979341_SNP | 46027, 46029- | 46073, 46087, | 46111, 46121- |
| | 46030, 46032, | 46096, 46183- | 46122, 46135, |
| | 46043, 46052, | 46222 | 46144, 46223- |
| | 46145-46182 | | 46264 |
| 12:40282009_GCTTCCCTTCC | 46265-46298 | 46299-46338 | 46339-46382 |
| CTTCCCTTCCCTTCC_G | | | |
| rs1226327150_REF | | | |
| 12:40282053_CCCTTCCCTTC | , | 46383 | 46384-46390 |
| CCTTCCCTTCCCTTCCCTTC | | | |
| CCTTCTCTT_C | | | |
| rs777452337_SNP | | | |
| 12:40282112_TCCCTC_T | 46391-46401 | 46383, 46402- | 46384, 46386- |
| rs1339001348_REF | | 46415 | 46388, 46416- |
| | | | 46432 |
| 12:40282398_G_T | 46433-46475 | 46476-46514 | 46515-46562 |
| rs11175847_REF | | | |
| 12:40282398_G_T | 46435, 46443, | 46477, 46479, | 46516, 46519, |
| rs11175847_SNP | 46445, 46447, | 46486, 46488- | 46524, 46529, |
| | 46450-46451, | 46489, 46492, | 46531, 46533- |
| | 46464, 46467, | 46504, 46506, | 46534, 46537, |
| | 46563-46589 | 46590-46612 | 46551, 46553, |
| | | | 46613-46639 |
| 12:40283044_TA_T | 46640-46644 | 46645-46649 | 46650-46654 |
| rs112859457_REF | | | |
| 12:40283045_A_T | 46641-46644 | 46645-46646, | 46650, 46652- |
| rs17466080_REF | | 46648-46649 | 46654 |
| 12:40283045_A_T | 46641-46644 | 46645-46646, | 46650, 46652- |
| rs17466080_SNP | | 46648-46649 | 46654 |
| 12:40283206_G_A | 46655-46702 | 46703-46752 | 46753-46804 |
| rs11175852_REF | | | |
| 12:40283206_G_A | 46657, 46666, | 46705, 46714- | 46755, 46759, |
| rs11175852_SNP | 46668, 46671, | 46715, 46717, | 46765-46766, |
| | 46680, 46693- | 46720, 46735, | 46768, 46771, |
| | 46695, 46698- | 46744-46745, | 46785, 46787, |
| | 46699, 46805- | 46748-46749, | 46797, 46800, |
| | 46842 | 46843-46882 | 46883-46924 |
| 12:40283227_A_C | 46657-46658, | 46705-46706, | 46755-46756, |
| rs10784486_REF | 46664, 46668, | 46712, 46717, | 46759, 46763, |
| | 46671, 46674, | 46720-46721, | 46768, 46771- |
| | 46925-46966 | 46723, 46735, | 46772, 46774, |
| | | 46967-47008 | 46778, 46787, |
| | | | 47009-47050 |
| 12:40283227_A_C | 46658, 46664, | 46706, 46712, | 46756, 46763, |
| rs10784486_SNP | 46674, 46925- | 46721, 46723, | 46772, 46774, |
| | 46927, 46932, | 46967-46968, | 46778, 47009, |
| | 46939, 46949, | 46974, 46981, | 47016, 47022, |
| | 46955, 47051- | 46983, 46997, | 47024, 47026, |
| | 47088 | 47089-47128 | 47129-47170 |
| 12:40283782_G_T | 47171-47217 | 47218-47248 | 47249-47288 |
| rs73108368_REF | | | |
| 12:40283782_G_T | 47172, 47175, | 47232, 47241, | 47255, 47279, |
| rs73108368_SNP | 47182, 47189, | 47246, 47310- | 47284, 47287, |
| | 47195, 47206, | 47319 | 47320-47336 |
| | 47212-47213, | | |
| | 47215, 47289- | | |
| | 47309 | | |
| 12:40284261_AT_A | 47337-47354 | 47355-47364 | 47365-47376 |
| rs398019201_REF | | | |
| 12:40284539_C_T | 47377-47405 | 47406-47431 | 47432-47464 |
| rs17519846_REF | | | |
| 12:40284539_C_T | 47382, 47390- | 47410-47411, | 47437-47438, |
| | 47391, 47393- | 47419, 47421, | 47440, 47451, |
| | 47394, 47398, | 47424, 47426, | 47456-47457, |
| rs17519846_SNP | 47400, 47402, | 47428, 47478 | 47459, 47461, |
| | 47465-47477 | | 47479-47483 |
| 12:40285452_G_A | 47484-47519 | 47520-47551 | 47552-47587 |
| rs17466150_REF | | | |
| 12:40285452_G_A | 47492-47493, | 47525-47526, | 47559-47561, |
| rs17466150_SNP | 47503, 47509, | 47540-47541, | 47576-47577, |
| | 47512, 47588- | 47544, 47611- | 47634-47658 |
| | 47610 | 47633 | |
| 12:40285467_T_A | 47484-47485, | 47520-47527, | 47552-47553, |
| rs11175862_REF | 47489-47493, | 47531, 47534- | 47556-47562, |
| | 47500, 47503- | 47537, 47539- | 47567, 47570- |
| | 47506, 47508- | 47541, 47544- | 47573, 47575- |
| | 47509, 47512- | 47546, 47550, | 47578, 47580- |
| | 47514, 47518, | 47673-47682 | 47582, 47586, |
| | 47659-47672 | | 47683-47694 |
| 12:40285467_T_A | 47504, 47506, | 47527, 47537, | 47562, 47573, |
| rs11175862_SNP | 47513-47514, | 47545-47546, | 47578, 47582, |
| | 47518, 47695- | 47550, 47722- | 47586, 47747- |
| | 47721 | 47746 | 47775 |
| 12:40286069_A_G | 47776-47822 | 47823-47870 | 47871-47920 |
| rs4310676_REF | | | |
| 12:40286069_A_G | 47782, 47784, | 47826, 47830, | 47874, 47880, |
| rs4310676_SNP | 47788, 47809- | 47832, 47836, | 47884, 47895, |
| | 47810, 47812, | 47858, 47860, | 47909-47910, |
| | 47819-47820, | 47868, 47870, | 47918, 47920, |
| | 47822, 47921- | 47959-47998 | 47999-48040 |
| | 47958 | | |
| 12:40287115_T_C | 48041-48082 | 48083-48115 | 48116-48157 |
| rs11564150_REF | | | |
| 12:40287115_T_C | 48049, 48060, | 48089, 48094, | 48128, 48132, |
| rs11564150_SNP | 48067, 48069, | 48104, 48107, | 48135, 48144, |
| | 48073, 48076, | 48110, 48112, | 48147, 48153, |
| | 48079, 48082, | 48115, 48196- | 48156-48157, |
| | 48158-48195 | 48235 | 48236-48277 |
| 12:40287139_A_G | 48278-48325 | 48093-48094, | 48127-48128, |
| rs4318033_REF | | 48326-48373 | 48131-48132, |
| | | | 48374-48421 |
| 12:40287139_A_G | 48278, 48283- | 48093, 48326, | 48127, 48131, |
| rs4318033_SNP | 48285, 48287, | 48331-48333, | 48374, 48378, |
| | 48298, 48302- | 48335, 48342, | 48381, 48384, |
| | 48303, 48309, | 48350, 48357, | 48391, 48398, |
| | 48312, 48422- | 48360, 48460- | 48405, 48408, |
| | 48459 | 48499 | 48500-48541 |
| 12:40287255_C_T | 48542-48575 | 48576-48609 | 48610-48645 |
| rs4473003_REF | | | |
| 12:40287255_C_T | 48555, 48561, | 48580, 48590, | 48611, 48616, |
| rs4473003_SNP | 48569, 48572, | 48595, 48603, | 48631, 48639, |
| | 48574, 48646- | 48608, 48671- | 48644, 48690- |
| | 48670 | 48689 | 48712 |
| 12:40287340_C_CT | 48713-48743 | 48744-48772 | 48773-48804 |
| rs1429478461_REF | | | |
| 12:40287340_C_CT | 48716, 48722, | 48753, 48763- | 48783-48784, |
| rs1429478461_SNP | 48731, 48734- | 48764, 48770, | 48791, 48795, |
| | 48735, 48741, | 48772, 48823- | 48801, 48804, |
| | 48805-48822 | 48838 | 48839-48858 |
| 12:40287550_T_TA | 48859-48862 | 48863-48865 | 48866-48869 |
| rs886049356_REF | | | |
| 12:40287684_A_G | 48870-48881 | 48882-48890 | 48891-48905 |
| rs11564270_REF | | | |
| 12:40287684_A_G | 48870-48871, | 48882-48883, | 48892, 48895, |
| rs11564270_SNP | 48873-48875, | 48886-48890, | 48898-48899, |
| | 48877-48881, | 48920-48927 | 48901, 48903, |
| | 48906-48919 | | 48905, 48928- |
| | | | 48941 |
| 12:40288395_C_G | 48942-48986 | 48987-49032 | 49033-49080 |
| rs17519881_REF | | | |
| 12:40288395_C_G | 48947, 48951, | 48992, 48996, | 49038, 49045, |
| rs17519881_SNP | 48959, 48963, | 49008, 49012, | 49055, 49059, |
| | 48967, 48970, | 49014, 49022, | 49061, 49063, |
| | 48976, 49081- | 49119-49158 | 49159-49200 |
| | 49118 | | |
| 12:40288788_G_T | 49201-49203 | , | 49204 |
| rs17466178_REF | | | |
| 12:40288788_G_T | 49203 | , | , |
| rs17466178_SNP | | | |
| 12:40290820_T_G | 49205-49236 | 49237-49257 | 49258-49285 |
| rs17466185_REF | | | |
| 12:40290820_T_G | 49205-49206, | 49239-49240, | 49258, 49262, |
| rs17466185_SNP | 49210-49211, | 49246-49247, | 49269-49270, |
| | 49215, 49218, | 49317-49345 | 49280, 49346- |
| | 49224, 49286- | | 49378 |
| | 49316 | | |
| 12:40291078_A_C | 49379-49383 | 49384-49390 | 49391-49398 |
| rs11175902_SNP | | | |
| 12:40291483_C_T | 49399-49444 | 49445-49480 | 49481-49522 |
| rs76033741_REF | | | |
| 12:40291483_C_T | 49400, 49403, | 49446, 49453, | 49481, 49484, |
| rs76033741_SNP | 49410, 49415, | 49458, 49463, | 49491, 49493, |
| | 49422-49423, | 49471, 49473, | 49497, 49512, |
| | 49430, 49434, | 49477, 49479, | 49519, 49521, |
| | 49439, 49444, | 49544-49562 | 49563-49588 |
| | 49523-49543 | | |
| 12:40292044_C_T | 49589-49635 | 49636-49685 | 49686-49737 |
| rs56325408_REF | | | |
| 12:40292044_C_T | 49590, 49592, | 49636, 49638, | 49686-49687, |
| rs56325408_SNP | 49597, 49599, | 49640, 49645, | 49689, 49691, |
| | 49613, 49621, | 49647, 49661, | 49698, 49712, |
| | 49624-49625, | 49672, 49674, | 49725, 49729, |
| | 49629, 49633, | 49678, 49685, | 49736-49737, |
| | 49738-49775 | 49776-49815 | 49816-49857 |
| 12:40292236_G_T | 49858-49902 | 49903-49946 | 49947-49996 |
| rs7957754_REF | | | |
| 12:40292236_G_T | 49862, 49872- | 49913, 49916- | 49958, 49963, |
| rs7957754_SNP | 49873, 49885, | 49917, 49927, | 49971, 49976- |
| | 49891-49892, | 49933-49935, | 49977, 49983- |
| | 49897-49899, | 49939, 49942- | 49984, 49989, |
| | 49997-50006 | 49943, 50007- | 49992-49993, |
| | | 50014 | 50015-50024 |
| 12:40292929_A_G | 50025-50037 | 50038-50046 | 50047-50058 |
| rs11175911_REF | | | |
| 12:40292929_A_G | 50031, 50034, | 50044, 50078- | 50054, 50058, |
| rs11175911_SNP | 50036-50037, | 50088 | 50089-50103 |
| | 50059-50077 | | |
| 12:40293043_A_G | 50104-50115 | 50116-50119 | 50120-50124 |
| rs12369882_REF | | | |
| 12:40293043_A_G | 50107, 50109- | 50116-50119, | 50120-50124, |
| rs12369882_SNP | 50110, 50114- | 50136-50144 | 50145-50155 |
| | 50115, 50125- | | |
| | 50135 | | |
| 12:40293233_C_T | 50156-50202 | 50203-50252 | 50253-50304 |
| rs11175913_REF | | | |
| 12:40293233_C_T | 50161-50163, | 50203, 50210, | 50253-50254, |
| rs11175913_SNP | 50168, 50177, | 50212-50213, | 50261, 50263, |
| | 50185, 50190- | 50218, 50227, | 50269, 50278- |
| | 50191, 50197, | 50235, 50240- | 50279, 50292- |
| | 50200, 50305- | 50241, 50247, | 50293, 50299, |
| | 50342 | 50343-50382 | 50383-50424 |
| 12:40293852_A_G | 50425-50456 | 50457-50473 | 50474-50497 |
| rs4483664_REF | | | |
| 12:40293852_A_G | 50436, 50438, | 50462-50463, | 50475, 50483- |
| rs4483664_SNP | 50442, 50444, | 50467, 50469, | 50484, 50488, |
| | 50446, 50453, | 50472, 50536- | 50495, 50568- |
| | 50498-50535 | 50567 | 50605 |
| 12:40293893_GCA_G | 50606-50639 | 50640-50660 | 50661-50684 |
| rs66487867_REF | | | |
| 12:40293893_GCA_G | 50607, 50616, | 50640, 50645, | 50661, 50666, |
| rs66487867_SNP | 50618, 50620- | 50647, 50649, | 50668, 50670, |
| | 50621, 50685- | 50658, 50695- | 50682, 50699- |
| | 50694 | 50698 | 50704 |
| 12:40293894_C_CAT | 50606-50618, | 50640-50647, | 50661, 50663- |
| rs1491426750_REF | 50620-50628, | 50649-50651, | 50670, 50672- |
| | 50630-50639 | 50653-50660 | 50684 |
| 12:40293894_C_CAT | 50607, 50616, | 50640, 50645, | 50661, 50666, |
| rs1491426750_SNP | 50618, 50620- | 50647, 50649, | 50668, 50670, |
| | 50621, 50688, | 50658, 50695- | 50682, 50700- |
| | 50690, 50692- | 50698, 50729- | 50702, 50704, |
| | 50693, 50705- | 50736 | 50737-50746 |
| | 50728 | | |
| 12:40293896_C_T | 50606-50612, | 50640-50642, | 50661, 50663- |
| rs5006481_REF | 50614-50618, | 50644-50647, | 50670, 50672- |
| | 50620-50622, | 50649-50651, | 50674, 50677- |
| | 50624-50628, | 50654-50658, | 50678, 50680- |
| | 50630-50631, | 50660 | 50682, 50684 |
| | 50634-50639 | | |
| 12:40293896_C_T | 50607, 50616, | 50640, 50645, | 50661, 50666, |
| rs5006481_SNP | 50618, 50620- | 50647, 50649, | 50668, 50670, |
| | 50621, 50685- | 50658, 50696- | 50682, 50699, |
| | 50687, 50689- | 50698 | 50701-50704 |
| | 50694 | | |
| 12:40294132_CATCTATCTAT | 50747-50753 | | 50754-50755 |
| CT_C | | | |
| rs139565575_REF | | | |
| 12:40294132_CATCTATCT_C | 50747-50753 | | 50754-50755 |
| rs150276517_REF | | | |
| 12:40294132_CATCT_C | 50747-50753 | | 50754-50755 |
| rs72446556_REF | | | |
| 12:40294132_C_CATCT | 50747-50753 | | 50754-50755 |
| rs1186032972_REF | | | |
| 12:40294279_A_G | 50756-50802 | 50803-50852 | 50853-50903 |
| rs12820920_REF | | | |
| 12:40294279_A_G | 50756-50757, | 50803-50804, | 50854, 50857, |
| rs12820920_SNP | 50759-50760, | 50807, 50813, | 50863, 50865- |
| | 50767-50768, | 50815-50816, | 50866, 50869, |
| | 50773, 50776, | 50821, 50829, | 50880, 50888, |
| | 50781, 50793, | 50838, 50843, | 50893, 50898, |
| | 50904-50941 | 50942-50981 | 50982-51023 |
| 12:40294893_T_C | 51024-51034 | 51035-51040 | 51041-51048 |
| rs7966550_REF | | | |
| 12:40294893_T_C | 51024-51025, | 51035-51036, | 51041, 51043- |
| rs7966550_SNP | 51028-51034, | 51038-51040, | 51044, 51046, |
| | 51049-51056 | 51057-51061 | 51048, 51062- |
| | | | 51069 |
| 12:40295119_A_G | 51070-51087 | | 51088-51089 |
| rs12314692_SNP | | | |
| 12:40295786_C_T | 51090-51136 | 51137-51184 | 51185-51236 |
| rs11175922_REF | | | |
| 12:40295786_C_T | 51091, 51096, | 51138, 51141, | 51185, 51187, |
| rs11175922_SNP | 51098, 51100, | 51144, 51146, | 51190, 51194, |
| | 51103-51105, | 51148, 51151- | 51196, 51201, |
| | 51118, 51125- | 51152, 51166, | 51216, 51222, |
| | 51126, 51237- | 51173-51174, | 51224-51225, |
| | 51268 | 51269-51296 | 51297-51330 |
| 12:40296087_T_C | 51331-51357 | 51358-51376 | 51377-51403 |
| rs73102746_REF | | | |
| 12:40296087_T_C | 51335, 51340, | 51360, 51362, | 51377, 51380, |
| rs73102746_SNP | 51346-51347, | 51366, 51370, | 51385, 51390, |
| | 51353, 51404- | 51438-51466 | 51395, 51467- |
| | 51437 | | 51499 |
| 12:40296180_T_C | 51500-51536 | 51537-51568 | 51569-51604 |
| rs10784497_REF | | | |
| 12:40296180_T_C | 51514, 51524, | 51559, 51563, | 51598, 51600, |
| rs10784497_SNP | 51530, 51533, | 51565, 51642- | 51602, 51677- |
| | 51605-51641 | 51676 | 51713 |
| 12:40296259_A_G | 51714-51749 | 51750-51773 | 51774-51799 |
| rs17443815_REF | | | |
| 12:40296259_A_G | 51716, 51726- | 51751, 51759- | 51775, 51778, |
| rs17443815_SNP | 51727, 51737, | 51760, 51768, | 51785, 51793, |
| | 51747, 51800- | 51771, 51838- | 51797, 51873- |
| | 51837 | 51872 | 51912 |
| 12:40296904_C_A | 51913-51959 | 51960-52005 | 52006-52057 |
| rs7957057_REF | | | |
| 12:40296904_C_A | 51914-51915, | 51961-51962, | 52007-52008, |
| rs7957057_SNP | 51923-51924, | 51967, 51970- | 52014, 52017- |
| | 51931, 51934, | 51971, 51977, | 52018, 52025, |
| | 51937, 51941, | 51987, 51993- | 52035, 52038, |
| | 51947-51948, | 51994, 52001, | 52050, 52056, |
| | 52058-52087 | 52088-52115 | 52116-52147 |
| 12:40297067_CTT_C | 52148-52193 | 52194-52243 | 52244-52295 |
| rs76607433_REF | | | |
| 12:40297067_CTT_C | 52163-52165, | 52212-52213, | 52256, 52264, |
| rs76607433_SNP | 52167, 52172, | 52215, 52220, | 52266, 52279- |
| | 52178, 52187, | 52227-52228, | 52280, 52291, |
| | 52189, 52296- | 52239, 52242, | 52294-52295, |
| | 52332 | 52333-52371 | 52372-52412 |
| 12:40297142_G_A | 52413-52452 | 52453-52478 | 52479-52511 |
| rs28365224_REF | | | |
| 12:40297142_G_A | 52420, 52423, | 52459, 52466, | 52487, 52490, |
| rs28365224_SNP | 52427, 52435, | 52472, 52474, | 52494, 52503, |
| | 52441, 52450, | 52477, 52523- | 52506, 52510, |
| | 52512-52522 | 52525 | 52526-52534 |
| 12:40298741_C_T | 52535-52541 | 52542-52553 | 52554-52566 |
| rs7962011_SNP | | | |
| 12:40299423_A_C | 52567-52572 | 52573-52574 | 52575-52577 |
| rs10878336_REF | | | |
| 12:40299423_A_C | 52567-52572, | 52573-52574, | 52575-52577, |
| rs10878336_SNP | 52578-52588 | 52589-52590 | 52591-52595 |
| 12:40299999_C_G | 52596-52643 | 52644-52693 | 52694-52745 |
| rs11175934_REF | | | |
| 12:40299999_C_G | 52605, 52610, | 52653, 52658, | 52703, 52708- |
| rs11175934_SNP | 52620, 52630, | 52668, 52678- | 52709, 52729- |
| | 52632, 52635, | 52679, 52684, | 52730, 52735, |
| | 52640-52643, | 52686, 52690- | 52737, 52742- |
| | 52746-52783 | 52691, 52693, | 52743, 52745, |
| | | 52784-52823 | 52824-52865 |
| 12:40300004_G_T | 52596-52599, | 52644-52647, | 52694-52697, |
| rs11175935_REF | 52601-52604, | 52649-52652, | 52699-52702, |
| | 52606-52607, | 52654-52655, | 52704-52705, |
| | 52609-52610, | 52657-52658, | 52707, 52709, |
| | 52614-52619, | 52662-52667, | 52713-52728, |
| | 52621-52634, | 52669-52677, | 52730-52734, |
| | 52636-52640, | 52679-52683, | 52736-52741, |
| | 52643, 52866- | 52685-52690, | 52743-52745, |
| | 52874 | 52692-52693, | 52881-52887 |
| | | 52875-52880 | |
| 12:40300004_G_T | 52615, 52618, | 52663, 52671, | 52719, 52722, |
| rs11175935_SNP | 52623, 52625, | 52673, 52687, | 52724, 52738, |
| | 52637, 52869- | 52692, 52878, | 52744, 52884, |
| | 52870, 52873- | 52926-52964 | 52887, 52965- |
| | 52874, 52888- | | 53005 |
| | 52925 | | |
| 12:40300216_T_C | 53006-53042 | 53043-53076 | 53077-53112 |
| rs17491061_REF | | | |
| 12:40300216_T_C | 53009, 53013, | 53046, 53049, | 53080, 53093, |
| rs17491061_SNP | 53021, 53028- | 53058, 53063- | 53098-53099, |
| | 53029, 53040, | 53064, 53147- | 53105, 53181- |
| | 53113-53146 | 53180 | 53218 |
| 12:40300597_G_A | 53219-53266 | 53267-53316 | 53317-53368 |
| rs11175941_REF | | | |
| 12:40300597_G_A | 53224, 53226, | 53270, 53273, | 53320, 53323, |
| rs11175941_SNP | 53238-53239, | 53275, 53287- | 53327, 53338- |
| | 53245-53246, | 53288, 53294- | 53339, 53345- |
| | 53250-53251, | 53295, 53299, | 53346, 53360, |
| | 53255, 53260, | 53309, 53312, | 53363, 53366, |
| | 53369-53406 | 53407-53446 | 53447-53488 |
| 12:40301293_G_A | 53489 | 53490-53492 | 53493-53499 |
| rs10878340_SNP | | | |
| 12:40303632_G_A | 53500-53533 | 53534-53557 | 53558-53589 |
| rs10784498_REF | | | |
| 12:40303632_G_A | 53500, 53512, | 53547, 53607- | 53565, 53573, |
| rs10784498_SNP | 53525-53526, | 53612 | 53582, 53613- |
| | 53590-53606 | | 53622 |
| 12:40303675_C_T | 53623-53670 | 53671-53715 | 53716-53763 |
| rs10878341_REF | | | |
| 12:40303675_C_T | 53626, 53633, | 53674, 53681, | 53719, 53731, |
| | | 53686, 53703, | |
| rs10878341_SNP | 53638, 53648, | 53707, 53802- | 53750, 53753, |
| | 53650, 53655, | 53841 | 53756, 53762, |
| | 53659, 53662, | | 53842-53883 |
| | 53668-53669, | | |
| | 53764-53801 | | |
| 12:40304688_C_T | 53884-53915 | 53916-53949 | 53950-53988 |
| rs10784499_REF | | | |
| 12:40304688_C_T | 53886-53887, | 53917, 53919, | 53951, 53953, |
| rs10784499_SNP | 53892, 53896, | 53925, 53927, | 53959, 53962, |
| | 53903, 53909, | 53929, 53937, | 53964, 53978, |
| | 53913, 53989- | 53943, 53947, | 53981-53982, |
| | 54023 | 54024-54043 | 53986, 54044- |
| | | | 54072 |
| 12:40304884_A_G | 54073 | | 54074 |
| rs17484286_REF | | | |
| 12:40304884_A_G | 54073 | | 54074 |
| rs17484286_SNP | | | |
| 12:40306303_CTT_C | 54075-54120 | 54121-54144 | 54145-54177 |
| rs146071397_REF | | | |
| 12:40306303_CTT_C | 54085, 54093, | 54131, 54136, | 54166, 54175, |
| rs146071397_SNP | 54101-54102, | 54144, 54216- | 54177, 54250- |
| | 54107, 54116, | 54249 | 54288 |
| | 54178-54215 | | |
| 12:40306782_C_T | 54289-54325 | 54326-54357 | 54358-54391 |
| rs10878343_REF | | | |
| 12:40306782_C_T | 54392-54421 | 54422-54443 | 54444-54471 |
| rs10878343_SNP | | | |
| 12:40307469_G_T | 54472-54488 | 54489-54493 | 54494-54501 |
| rs11564180_REF | | | |
| 12:40307469_G_T | 54473, 54481, | | 54501 |
| rs11564180_SNP | 54502-54504 | | |
| 12:40307586_G_A | 54505-54510 | | |
| rs11175958_REF | | | |
| 12:40307927_A_G | 54511-54535 | 54536-54562 | 54563-54591 |
| rs11175959_REF | | | |
| 12:40307927_A_G | 54511, 54513, | 54536-54537, | 54563-54565, |
| rs11175959_SNP | 54518, 54532, | 54539, 54544, | 54567, 54572, |
| | 54534, 54592- | 54558, 54612- | 54634-54657 |
| | 54611 | 54633 | |
| 12:40308029_C_T | , | 54658-54659 | 54660-54663 |
| rs113772607_SNP | | | |
| 12:40308362_C_T | 54664-54697 | 54698-54726 | 54727-54758 |
| rs11564149_REF | | | |
| 12:40308362_C_T | 54670, 54686, | 54702, 54717, | 54732, 54747, |
| rs11564149_SNP | 54688, 54690, | 54784-54805 | 54749, 54806- |
| | 54697, 54759- | | 54829 |
| | 54783 | | |
| 12:40308969_C_T | 54830-54876 | 54877-54924 | 54925-54976 |
| rs11175963_REF | | | |
| 12:40308969_C_T | 54830, 54832, | 54890, 54893, | 54934, 54940, |
| rs11175963_SNP | 54844, 54848, | 54895, 54906- | 54943, 54945, |
| | 54859, 54861, | 54907, 54909, | 54956-54957, |
| | 54863-54864, | 54912-54913, | 54959, 54962, |
| | 54867, 54869, | 55009-55024 | 54968, 54970, |
| | 54977-55008 | | 55025-55051 |
| 12:40309044_A_T | 55052-55063 | 55064-55073 | 55074-55085 |
| rs7305344_REF | | | |
| 12:40309044_A_T | 55052-55053, | 55064-55065, | 55074-55075, |
| rs7305344_SNP | 55055, 55058- | 55067-55068, | 55077-55078, |
| | 55059, 55086- | 55070, 55093- | 55083, 55098- |
| | 55092 | 55097 | 55104 |
| 12:40309109_G_A | 55105-55140 | 55141-55181 | 55182-55229 |
| rs7133914_REF | | | |
| 12:40309109_G_A | 55120, 55123, | 55149, 55162- | 55191, 55204- |
| rs7133914_SNP | 55126, 55230- | 55164, 55176, | 55207, 55211, |
| | 55260 | 55261-55287 | 55288-55318 |
| 12:40309185_G_A | 15754, 15758, | 15800, 15806, | 15849, 15851, |
| rs11175964_REF | 15769, 15786, | 15817, 15835, | 15869, 15887, |
| | 15903, 15910, | 15845, 15949, | 15897, 15999, |
| | 15916, 55319- | 15956, 15963, | 16003, 16007, |
| | 55354 | 15975, 55355- | 16014, 16026, |
| | | 55395 | 55396-55437 |
| 12:40309185_G_A | 55332, 55336, | 55357, 55362, | 55397, 55401, |
| rs11175964_SNP | 55339, 55341- | 55366, 55371, | 55406, 55411, |
| | 55342, 55350- | 55376, 55381, | 55416, 55424, |
| | 55351, 55354, | 55387, 55391- | 55428, 55433, |
| | 55438-55475 | 55392, 55395, | 55436-55437, |
| | | 55476-55515 | 55516-55557 |
| 12:40309285_C_CGT | 55558-55577 | 55578-55596 | 55597-55617 |
| rs397805328_REF | | | |
| 12:40309350_C_A | 55618-55632 | 55633-55637 | 55638-55645 |
| rs11175966_REF | | | |
| 12:40309350_C_A | 55618, 55622- | 55633, 55635 | 55638, 55640, |
| rs11175966_SNP | 55623, 55631- | | 55642 |
| | 55632, 55646 | | |
| 12:40310063_A_G | 55647-55652 | 55653-55657 | 55658-55663 |
| rs10784501_REF | | | |
| 12:40310063_A_G | 55647-55649, | 55653-55657, | 55658-55660, |
| rs10784501_SNP | 55651-55652, | 55675-55677 | 55662-55663, |
| | 55664-55674 | | 55678-55682 |
| 12:40310755_T_C | 55683-55725 | 55726-55766 | 55767-55811 |
| rs41286474_REF | | | |
| 12:40310755_T_C | 55688-55689, | 55730, 55732- | 55771, 55773, |
| rs41286474_SNP | 55693-55694, | 55733, 55737, | 55778, 55781, |
| | 55697-55698, | 55740, 55745, | 55785, 55790, |
| | 55702, 55705, | 55748, 55758, | 55801-55803, |
| | 55717, 55812- | 55850-55887 | 55888-55929 |
| | 55849 | | |
| 12:40311346_CT_C | 55930-55947 | 55948-55958 | 55959-55971 |
| rs869212703_REF | | | |
| 12:40311346_CT_C | 55940, 55945 | | 55969 |
| rs869212703_SNP | | | |
| 12:40311356_T_A | 55940, 55945, | 55977 | 55969, 55978- |
| rs2069228_REF | 55972-55976 | | 55979 |
| 12:40311356_T_A | 55940, 55945, | 55977 | 55969, 55978- |
| rs2069228_SNP | 55972-55976 | | 55979 |
| 12:40311898_C_G | 55980-55989 | 55990-55993 | 55994-56000 |
| rs10784503_REF | | | |
| 12:40311898_C_G | 55981, 55983, | 55990-55991, | 55995-55996, |
| rs10784503_SNP | 55985, 55987, | 55993, 56006 | 55998-55999, |
| | 55989, 56001- | | |
| | 56005 | | 56007-56009 |
| 12:40311964_G_GT | 56010-56028 | 56029-56044 | 56045-56062 |
| rs386376250_REF | | | |
| 12:40311964_G_GT | 56027 | 56040 | 56061 |
| rs386376250_SNP | | | |
| 12:40312033_C_T | 56063-56100 | 56101-56134 | 56135-56172 |
| rs11564128_REF | | | |
| 12:40312033_C_T | 56073, 56080, | 56108, 56111, | 56140-56141, |
| rs11564128_SNP | 56082, 56090- | 56114, 56116, | 56145, 56149, |
| | 56091, 56094, | 56123, 56130, | 56161, 56168, |
| | 56173-56194 | 56195-56202 | 56203-56218 |
| 12:40312573_G_T | 56219-56265 | 56266-56312 | 56313-56362 |
| rs2896975_REF | | | |
| 12:40312573_G_T | 56219, 56229, | 56270, 56277, | 56314, 56318, |
| rs2896975_SNP | 56243-56244, | 56292, 56299, | 56325, 56340, |
| | 56251, 56257, | 56305, 56309, | 56342, 56358, |
| | 56260, 56263- | 56312, 56401- | 56361-56362, |
| | 56265, 56363- | | |
| | 56400 | 56440 | 56441-56482 |
| 12:40312729_G_A | 56483-56530 | 56531-56580 | 56581-56632 |
| rs7302503_REF | | | |
| 12:40312729_G_A | 56484, 56488, | 56532, 56541, | 56591, 56596, |
| rs7302503_SNP | 56493, 56498, | 56546, 56548, | 56598, 56600, |
| | 56501, 56519, | 56568, 56570, | 56619-56620, |
| | 56521, 56528- | 56577-56580, | 56629-56632, |
| | 56530, 56633- | 56671-56710 | 56711-56752 |
| | 56670 | | |
| 12:40312802_T_C | 56753-56766 | 56767-56775 | 56776-56787 |
| rs7137160_REF | | | |
| 12:40312802_T_C | 56753, 56755, | 56767-56769, | 56776-56777, |
| rs7137160_SNP | 56757-56758, | 56771-56775, | 56779, 56781- |
| | 56760-56761, | 56806-56813 | 56782, 56784- |
| | 56763-56766, | | 56787, 56814- |
| | 56788-56805 | | 56827 |
| 12:40312938_TTAAGAGA_T | 56828-56858 | 56859-56900 | 56901-56948 |
| rs369794148_REF | | | |
| 12:40312938_TTAAGAGA_T | 56829, 56833- | 56860, 56867, | 56901, 56904, |
| rs369794148_SNP | 56834, 56848, | 56884, 56896, | 56911, 56921, |
| | 56857, 56949- | 56987-57026 | 56944, 57027- |
| | 56986 | | 57068 |
| 12:40312990_GA_G | 57069-57109 | 57110-57150 | 57151-57196 |
| rs34709132_REF | | | |
| 12:40312990_GA_G | 57070-57071, | 57110, 57112- | 57151, 57154- |
| rs34709132_SNP | 57075-57077, | 57113, 57117, | 57155, 57160, |
| | 57088, 57091, | 57120, 57133, | 57163, 57165, |
| | 57094, 57100, | 57136, 57141, | 57184-57186, |
| | 57197-57228 | 57229-57260 | 57261-57296 |
| 12:40313399_A_G | 57297-57338 | 57339-57369 | 57370-57409 |
| rs10467144_REF | | | |
| 12:40313399_A_G | 57309, 57314, | 57345, 57350, | 57378, 57380, |
| rs10467144_SNP | 57317, 57321, | 57353, 57362, | 57385, 57397, |
| | 57327, 57334, | 57364, 57367, | 57402, 57405, |
| | 57410-57447 | 57448-57486 | 57409, 57487- |
| | | | 57527 |
| 12:40313476_C_T | 57528-57572 | 57573-57618 | 57619-57668 |
| rs11175985_REF | | | |
| 12:40313476_C_T | 57534, 57545, | 57578, 57591, | 57624-57625, |
| rs11175985_SNP | 57547-57548, | 57593, 57596- | 57640, 57642, |
| | 57551, 57565, | 57597, 57611, | 57646-57647, |
| | 57570, 57669- | 57616, 57706- | 57661, 57666, |
| | 57705 | 57738 | 57739-57775 |
| 12:40313594_T_G | 57776-57801 | 57802-57815 | 57816-57836 |
| rs10784504_REF | | | |
| 12:40313594_T_G | 57779, 57781, | 57804, 57808, | 57819-57820, |
| rs10784504_SNP | 57784-57785, | 57811-57812, | 57826, 57829- |
| | 57792-57793, | 57869-57891 | 57830, 57892- |
| | 57795, 57798- | | 57919 |
| | 57799, 57837- | | |
| | 57868 | | |
| 12:40313687_G_GT | 57920-57926 | 57927-57928 | 57929-57930 |
| rs57421387_REF | | | |
| 12:40313687_G_GT | 57923 | 57927 | 57930 |
| rs57421387_SNP | | | |
| 12:40315043_A_T | 57931-57946 | 57947-57954 | 57955-57964 |
| rs7973058_REF | | | |
| 12:40315043_A_T | 57933, 57935- | 57947-57948, | 57956-57957, |
| rs7973058_SNP | 57936, 57939, | 57951, 57953- | 57960-57961, |
| | 57945, 57965- | 57954, 57976- | 57964, 57979- |
| | 57975 | 57978 | 57983 |
| 12:40315090_T_C | 57984-58030 | 58031-58080 | 58081-58132 |
| rs1427267_REF | | | |
| 12:40315090_T_C | 57984-57985, | 58032, 58034- | 58082, 58084- |
| rs1427267_SNP | 57987-57988, | 58035, 58044, | 58085, 58093, |
| | 57992, 57996, | 58047, 58056- | 58095, 58108, |
| | 57999, 58008- | 58057, 58059, | 58110, 58117, |
| | 58009, 58011, | 58074, 58076, | 58126, 58128, |
| | 58133-58168 | 58169-58208 | 58209-58250 |
| 12:40315120_C_T | 58251-58296 | 58297-58344 | 58345-58396 |
| rs1427266_REF | | | |
| 12:40315120_C_T | 58253, 58257- | 58297, 58299, | 58345-58346, |
| rs1427266_SNP | 58258, 58267- | 58303, 58305- | 58349, 58353, |
| | 58268, 58276, | 58306, 58314- | 58355, 58366, |
| | 58279-58280, | 58315, 58323, | 58374, 58377, |
| | 58284, 58289, | 58327, 58332, | 58379, 58384, |
| | 58397-58425 | 58426-58453 | 58454-58487 |
| 12:40315383_A_G | 58488-58518 | 58519-58544 | 58545-58582 |
| rs721713_REF | | | |
| 12:40315383_A_G | 58494, 58498, | 58524, 58526- | 58547, 58553, |
| rs721713_SNP | 58500, 58502, | 58527, 58529, | 58560, 58562, |
| | 58504, 58510, | 58531, 58536, | 58564, 58572, |
| | 58512-58513, | 58538-58539, | 58574-58575, |
| | 58516, 58518, | 58542, 58544, | 58580, 58582, |
| | 58583-58620 | 58621-58652 | 58653-58688 |
| 12:40315516_G_A | 58689-58735 | 58736-58785 | 58786-58837 |
| rs721712_REF | | | |
| 12:40315516_G_A | 58694, 58697- | 58741, 58744, | 58791, 58793, |
| rs721712_SNP | 58698, 58704, | 58751, 58756, | 58795, 58802, |
| | 58709, 58712, | 58759-58760, | 58810, 58816, |
| | 58717, 58723, | 58765, 58771, | 58818, 58823, |
| | 58727, 58732, | 58775, 58781, | 58827, 58833, |
| | 58838-58875 | 58876-58915 | 58916-58957 |
| 12:40315526_G_A | 58689-58693, | 58736-58740, | 58786-58790, |
| rs721711_REF | 58695-58699, | 58742-58746, | 58792-58798, |
| | 58702-58703, | 58749-58750, | 58800-58801, |
| | 58705, 58710- | 58752, 58757- | 58803, 58808- |
| | 58712, 58715, | 58760, 58763, | 58811, 58814, |
| | 58717, 58719- | 58765, 58767- | 58816, 58819- |
| | 58720, 58722, | 58768, 58770, | 58820, 58822, |
| | 58725, 58727- | 58773-58775, | 58825-58827, |
| | 58728, 58731, | 58777, 58780, | 58829, 58832, |
| | 58733-58735, | 58783-58785, | 58835-58837, |
| | 58958-58977 | 58978-58997 | 58998-59017 |
| 12:40315526_G_A | 58695, 58710- | 58742, 58757- | 58792, 58798, |
| rs721711_SNP | 58711, 58728, | 58758, 58774, | 58808-58809, |
| | 58733, 58963, | 58783, 58983, | 58826, 59001, |
| | 58968, 58974- | 58988, 58991, | 59003, 59008, |
| | 58975, 58977, | 58995, 58997, | 59011, 59017, |
| | 59018-59051 | 59052-59081 | 59082-59119 |
| 12:40315611_T_A | 59120-59160 | 59161-59200 | 59201-59245 |
| rs721709_REF | | | |
| 12:40315611_T_A | 59126, 59139, | 59167, 59182- | 59208, 59225, |
| rs721709_SNP | 59142-59144, | 59183, 59190, | 59232, 59234, |
| | 59151, 59246- | 59196, 59281- | 59240, 59244, |
| | 59280 | 59315 | 59316-59354 |
| 12:40315697_T_G | 59355-59399 | 59400-59443 | 59444-59490 |
| rs4768227_REF | | | |
| 12:40315697_T_G | 59361, 59374- | 59403, 59407, | 59445, 59448, |
| | 59375, 59377, | 59420, 59424, | 59452, 59465- |
| | 59379, 59395, | | |
| rs4768227_SNP | 59399, 59491- | 59440, 59529- | 59466, 59470, |
| | 59528 | 59568 | 59569-59610 |
| 12:40315806_AT_A | 59611-59612 | | |
| rs398098159_REF | | | |
| 12:40315806_AT_A | 59611-59617 | | 59618-59619 |
| rs398098159_SNP | | | |
| 12:40315822_G_T | 59611-59613, | 59642-59650 | 59651-59663 |
| rs4767969_REF | 59620-59641 | | |
| 12:40315822_G_T | 59620-59621, | | 59667-59668 |
| rs4767969_SNP | 59631, 59638, | | |
| | 59664-59666 | | |
| 12:40316155_G_A | 59669-59710 | 59711-59753 | 59754-59798 |
| rs10878356_REF | | | |
| 12:40316155_G_A | 59670, 59675, | 59712, 59718, | 59755, 59762- |
| rs10878356_SNP | 59694-59695, | 59737-59738, | 59763, 59782, |
| | 59799-59835 | 59836-59870 | 59871-59907 |
| 12:40317268_C_T | 59908-59917 | 59918-59922 | 59923-59932 |
| rs10784506_REF | | | |
| 12:40317268_C_T | 59911-59912, | 59918, 59921- | 59924, 59928- |
| rs10784506_SNP | 59915 | 59922 | 59930, 59932 |
| 12:40317289_G_A | 59908, 59911- | 59918-59919, | 59924, 59926, |
| rs10878358_REF | 59913, 59915- | 59921-59922, | 59928-59932, |
| | 59916, 59933- | 59961-59970 | 59971-59987 |
| | 59960 | | |
| 12:40317289_G_A | 59908, 59913, | 59919, 59998- | 59926, 59931, |
| rs10878358_SNP | 59916, 59950, | 60001 | 60002-60007 |
| | 59955, 59988- | | |
| | 59997 | | |
| 12:40317292_A_T | 59933-59960, | 59918-59919, | 59924, 59926, |
| rs7132073_REF | 60008-60009 | 59961-59970, | 59928, 59931, |
| | | 60010 | 59971-59987, |
| | | | 60011 |
| 12:40317292_A_T | 59950, 59958, | 59919, 59970, | 59926, 59931, |
| rs7132073_SNP | 59960, 60012- | 60039-60049 | 59975, 59977, |
| | 60038 | | 59979, 60050- |
| | | | 60066 |
| 12:40318196_C_A | 60067-60112 | 60113-60157 | 60158-60204 |
| rs1427265_REF | | | |
| 12:40318196_C_A | 60068, 60071, | 60116, 60126, | 60162, 60172, |
| rs1427265_SNP | 60075, 60078, | 60129, 60131, | 60175, 60191, |
| | 60083, 60085, | 60147, 60243- | 60194, 60282- |
| | 60095, 60101, | 60281 | 60322 |
| | 60107, 60205- | | |
| | 60242 | | |
| 12:40318557_C_T | 60323-60368 | 60369-60407 | 60408-60455 |
| rs17466339_REF | | | |
| 12:40318557_C_T | 60328, 60330, | 60372, 60384- | 60415-60416, |
| rs17466339_SNP | 60338, 60341, | 60385, 60391, | 60429-60430, |
| | 60348, 60355, | 60397, 60401, | 60436, 60443, |
| | 60359, 60456- | 60481-60499 | 60447, 60500- |
| | 60480 | | 60522 |
| 12:40318773_A_C | 60523-60548 | 60549-60555 | 60556-60568 |
| rs2114569_REF | | | |
| 12:40318773_A_C | 60524, 60526- | 60549, 60552, | 60556, 60559, |
| rs2114569_SNP | 60527, 60529- | 60605-60628 | 60566, 60629- |
| | 60530, 60533- | | 60662 |
| | 60535, 60541- | | |
| | 60542, 60569- | | |
| | 60604 | | |
| 12:40318818_G_A | 60663-60710 | 60711-60759 | 60760-60811 |
| rs2114568_REF | | | |
| 12:40318818_G_A | 60667, 60670, | 60735, 60742- | 60783, 60785, |
| rs2114568_SNP | 60687, 60694- | 60743, 60746, | 60792-60793, |
| | 60695, 60698- | 60751, 60753- | 60802, 60804- |
| | 60699, 60705- | 60755, 60758, | 60806, 60809- |
| | 60706, 60709, | 60850-60888 | 60810, 60889- |
| | 60812-60849 | | 60929 |
| 12:40319201_G_A | 60930-60967 | 60968-61002 | 61003-61043 |
| rs2162469_REF | | | |
| 12:40319201_G_A | 60943, 61044- | 61075-61084 | 61085-61100 |
| rs2162469_SNP | 61074 | | |
| 12:40319398_G_A | 61101-61148 | 61149-61198 | 61199-61250 |
| rs10784509_REF | | | |
| 12:40319398_G_A | 61107, 61118- | 61155, 61166- | 61216, 61224- |
| rs10784509_SNP | 61119, 61122, | 61167, 61174- | 61229, 61232, |
| | 61126-61127, | 61177, 61180, | 61236, 61246, |
| | 61130, 61134, | 61184, 61194, | 61329-61370 |
| | 61144, 61146, | 61289-61328 | |
| | 61251-61288 | | |
| 12:40319758_T_G | 61371-61417 | 61418-61457 | 61458-61504 |
| rs1896254_REF | | | |
| 12:40319758_T_G | 61371, 61375, | 61418, 61421, | 61458, 61461, |
| rs1896254_SNP | 61378, 61383, | 61427, 61431, | 61487-61488, |
| | 61387, 61405, | 61445, 61449, | 61493, 61498, |
| | 61410, 61413, | 61543-61582 | 61501, 61583- |
| | 61416, 61505- | | 61624 |
| | 61542 | | |
| 12:40319784_CA_C | 61625-61627 | 61628-61630 | 61631-61634 |
| rs5797667_REF | | | |
| 12:40319784_CA_C | 61626 | 61629 | 61632, 61634 |
| rs5797667_SNP | | | |
| 12:40319786_AT_A | 61626 | 61629 | 61632, 61634 |
| rs373676201_REF | | | |
| 12:40319793_T_C | 61635-61636 | 61637-61638 | 61639-61640 |
| rs1896253_REF | | | |
| 12:40319793_T_C | 61635-61636 | 61637-61638 | 61639-61640 |
| rs1896253_SNP | | | |
| 12:40319957_T_C | 61641-61644 | | 61645 |
| rs1896252_REF | | | |
| 12:40319957_T_C | 61641-61644, | 61647 | 61645, 61648- |
| rs1896252_SNP | 61646 | | 61650 |
| 12:40320032_C_A | 19471, 19475, | 19516-19517, | 19558-19559, |
| rs1427263_REF | 19477, 19486, | 19520, 19522, | 19561, 19564, |
| | 19491, 19620, | 19530, 19644, | 19574, 19662, |
| | 19622, 61651- | 19646-19647, | 19672, 19674- |
| | 61688 | 61689-61730 | 19675, 61731- |
| | | | 61773 |
| 12:40320032_C_A | 61657, 61660, | 61692, 61698, | 61733, 61739, |
| rs1427263_SNP | 61665, 61668, | 61703, 61706, | 61747, 61756, |
| | 61676, 61678, | 61716, 61718, | 61758, 61760, |
| | 61683, 61686- | 61723, 61728- | 61765, 61770, |
| | 61688, 61774- | 61730, 61812- | 61772-61773, |
| | 61811 | 61851 | 61852-61893 |
| 12:40320071_A_G | 61894-61898 | 61899-61900 | 61901-61904 |
| rs11176013_REF | | | |
| 12:40320071_A_G | 61894-61898, | 61899-61900 | 61901-61904, |
| rs11176013_SNP | 61905-61911 | | 61912 |
| 12:40320099_T_A | 19709, 19712, | 19739, 19752, | 19779, 19790, |
| rs11564148_REF | 19823, 19825, | 19867, 19880, | 19793-19794, |
| | 19838, 19841, | 19884, 19888, | 19913, 19920, |
| | 19958, 19966, | 20001, 20009, | 19930, 19934, |
| | 19969, 19974, | 20017, 20024, | 20037, 20047, |
| | 19979, 20087, | 20029, 20129, | 20070, 20075, |
| | 20093, 20106, | 20134, 20139, | 20077, 20173, |
| | 20116, 61913- | 20152, 20161, | 20178, 20184, |
| | 61931 | 61932-61951 | 20199, 20207, |
| | | | 61952-61977 |
| 12:40320099_T_A | 19958, 19966, | 20001, 20009, | 20037, 20047, |
| rs11564148_SNP | 19969, 19974, | 20017, 20024, | 20070, 20075, |
| | 19979, 61917, | 20029, 62014- | 20077, 61966, |
| | 61929, 61931, | 62044 | 61969, 62045- |
| | 61978-62013 | | 62081 |
| 12:40320207_A_G | 62082-62103 | 62104-62115 | 62116-62131 |
| rs11564205_REF | | | |
| 12:40320207_A_G | 62084, 62092, | 62165-62187 | 62188-62212 |
| rs11564205_SNP | 62132-62164 | | |
| 12:40320386_G_A | 62213-62228 | 62229-62241 | 62242-62257 |
| rs10748030_REF | | | |
| 12:40320386_G_A | 62215, 62221, | 62231, 62234, | 62244, 62247, |
| rs10748030_SNP | 62223, 62225- | 62236, 62239, | 62249, 62251, |
| | 62226, 62258- | 62265-62268 | 62255, 62269- |
| | 62264 | | 62274 |
| 12:40320464_T_C | 62275-62284 | 62285-62296 | 62297-62310 |
| rs10748031_REF | | | |
| 12:40320464_T_C | 62275-62277, | 62285-62286, | 62298-62300, |
| rs10748031_SNP | 62280-62281, | 62288, 62290, | 62303, 62305, |
| | 62284, 62311- | 62292-62293, | 62307, 62310, |
| | 62336 | 62295, 62337- | 62362-62386 |
| | | 62361 | |
| 12:40320465_G_T | 62275-62283, | 62285-62291, | 62297-62299, |
| rs10748032_REF | 62387-62388 | 62293-62296, | 62301-62310, |
| | | 62389-62390 | 62391-62392 |
| 12:40320465_G_T | 62275-62277, | 62286, 62288, | 62298-62299, |
| rs10748032_SNP | 62280-62281, | 62290, 62293, | 62305, 62307, |
| | 62387, 62393- | 62295, 62390, | 62310, 62392, |
| | 62419 | 62420-62446 | 62447-62473 |
| 12:40320530_T_C | 62474-62518 | 62519-62558 | 62559-62601 |
| rs11564177_REF | | | |
| 12:40320530_T_C | 62476, 62481, | 62531, 62545, | 62571, 62587, |
| rs11564177_SNP | 62489, 62506, | 62548, 62640- | 62590, 62598, |
| | 62513-62514, | 62679 | 62680-62721 |
| | 62518, 62602- | | |
| | 62639 | | |
| 12:40320593_C_T | 62722-62762 | 62763-62790 | 62791-62828 |
| rs10748033_REF | | | |
| 12:40320593_C_T | 62724, 62726, | 62764, 62768, | 62792, 62797, |
| rs10748033_SNP | 62735-62736, | 62772-62773, | 62802-62803, |
| | 62746, 62753, | 62785, 62848- | 62805, 62822, |
| | 62829-62847 | 62860 | 62861-62877 |
| 12:40320699_T_G | 62878-62895 | 62896-62913 | 62914-62933 |
| rs10735931_REF | | | |
| 12:40320699_T_G | 62880-62881, | 62899-62900, | 62915, 62918, |
| rs10735931_SNP | 62884, 62888, | 62902, 62906, | 62920, 62926, |
| | 62892, 62934- | 62910, 62953- | 62930, 62968- |
| | 62952 | 62967 | 62984 |
| 12:40320816_C_T | 62985-63024 | 63025-63058 | 63059-63102 |
| rs10748034_REF | | | |
| 12:40320816_C_T | 62994, 63003, | 63033, 63044, | 63068, 63072, |
| rs10748034_SNP | 63008, 63012- | 63047, 63053, | 63090, 63096, |
| | 63013, 63019, | 63055, 63126- | 63098, 63100, |
| | 63103-63125 | 63142 | 63143-63163 |
| 12:40320983_A_T | 63164-63209 | 63210-63255 | 63256-63306 |
| rs10878368_REF | | | |
| 12:40320983_A_T | 63176, 63179- | 63217, 63223, | 63263, 63269, |
| rs10878368_SNP | 63181, 63190- | 63226-63228, | 63272, 63274, |
| | 63191, 63199- | 63237-63238, | 63283-63284, |
| | 63200, 63202- | 63246, 63249, | 63293, 63298- |
| | 63203, 63307- | 63344-63380 | 63300, 63381- |
| | 63343 | | 63421 |
| 12:40321211_T_A | 63422-63424 | 63425-63427 | 63428-63431 |
| rs7307276_REF | | | |
| 12:40321211_T_A | 63422-63424 | 63425-63427 | 63428-63431 |
| rs7307276_SNP | | | |
| 12:40321446_T_C | 63432-63479 | 63480-63529 | 63530-63581 |
| rs4768229_REF | | | |
| 12:40321446_T_C | 63436, 63440- | 63484, 63488, | 63532, 63535, |
| rs4768229_SNP | 63441, 63450, | 63493, 63499, | 63539, 63544, |
| | 63461, 63464, | 63506, 63511, | 63553, 63558, |
| | 63467-63468, | 63517-63518, | 63569-63570, |
| | 63477-63478, | 63527-63528, | 63579-63580, |
| | 63582-63619 | 63620-63659 | 63660-63701 |
| 12:40321500_G_A | 63702-63731 | 63732-63761 | 63762-63793 |
| rs4768230_REF | | | |
| 12:40321500_G_A | 63711, 63720, | 63739, 63741, | 63769, 63771, |
| rs4768230_SNP | 63725, 63728, | 63750, 63758, | 63778, 63781, |
| | 63730, 63794- | 63760, 63817- | 63791, 63838- |
| | 63816 | 63837 | 63860 |
| 12:40321508_C_CA | 63704-63705, | 63734-63735, | 63764-63765, |
| rs397968969_REF | 63707-63709, | 63737-63738, | 63767-63768, |
| | 63712-63713, | 63742-63743, | 63770, 63772- |
| | 63716, 63721- | 63746, 63751- | 63773, 63776, |
| | 63722, 63726- | 63752, 63754, | 63782-63783, |
| | 63727, 63729, | 63756-63757, | 63785, 63787- |
| | 63731, 63861- | 63759, 63761, | 63788, 63790, |
| | 63863 | 63864-63867 | 63792-63793, |
| | | | 63868-63871 |
| 12:40321508_C_CA | 63709, 63713, | 63735, 63743, | 63765, 63768, |
| rs397968969_SNP | 63862-63863 | 63864-63866 | 63773, 63868- |
| | | | 63869, 63871 |
| 12:40321575_G_A | 63872-63909 | 63910-63943 | 63944-63980 |
| rs11564204_REF | | | |
| 12:40321575_G_A | 63873, 63875, | 63911, 63917, | 63945, 63947, |
| rs11564204_SNP | 63882, 63892, | 63933, 64010- | 63953, 63956, |
| | 63899, 63981- | 64038 | 64039-64069 |
| | 64009 | | |
| 12:40321788_C_T | 64070-64114 | 64115-64157 | 64158-64203 |
| rs4423250_REF | | | |
| 12:40321788_C_T | 64083, 64086, | 64128, 64134, | 64171, 64177, |
| rs4423250_SNP | 64089, 64091, | 64136, 64241- | 64192, 64194, |
| | 64102, 64105- | 64266 | 64267-64301 |
| | 64106, 64204- | | |
| | 64240 | | |
| 12:40322213_C_T | 64302-64321 | 64322-64323 | 64324-64329 |
| rs7137665_REF | | | |
| 12:40322458_T_C | 21565, 21569, | 21580, 64351- | 21581, 21583, |
| rs10878371_REF | 21573, 21575, | 64365 | 64366-64385 |
| | 21577, 64330- | | |
| | 64350 | | |
| 12:40322458_T_C | 64386-64422 | 64423-64454 | 64455-64488 |
| rs10878371_SNP | | | |
| 12:40322672_T_C | 64489-64490 | | |
| rs11176022_REF | | | |
| 12:40322672_T_C | 64489-64494 | | 64495-64496 |
| rs11176022_SNP | | | |
| 12:40322834_CAT_C | 64497-64542 | 64543-64589 | 64590-64641 |
| rs60750674_REF | | | |
| 12:40322834_CAT_C | 64498, 64500, | 64543, 64545, | 64591, 64593, |
| rs60750674_SNP | 64506-64507, | 64551-64552, | 64601, 64609, |
| | 64516-64517, | 64560, 64562, | 64611, 64622, |
| | 64526, 64531, | 64572, 64578, | 64628, 64633, |
| | 64642-64680 | 64681-64721 | 64722-64764 |
| 12:40322892_A_G | 64765-64812 | 64813-64860 | 64861-64912 |
| rs10878372_REF | | | |
| 12:40322892_A_G | 64770, 64772, | 64818, 64820, | 64866, 64868, |
| rs10878372_SNP | 64775, 64784, | 64823, 64831, | 64880-64881, |
| | 64786, 64789, | 64833, 64836, | 64883, 64886- |
| | 64793-64794, | 64840, 64852, | 64887, 64891, |
| | 64803, 64811, | 64856, 64859, | 64903, 64908, |
| | 64913-64950 | 64951-64990 | 64991-65032 |
| 12:40323798_CTTT_C | 65033-65044 | 65045-65056 | 65057-65070 |
| rs398055492_REF | | | |
| 12:40323798_CTT_C | 65033-65044 | 65045-65056 | 65057-65070 |
| rs761931834_REF | | | |
| 12:40323798_CT_C | 65033-65044 | 65045-65056 | 65057-65070 |
| rs1415940047_REF | | | |
| 12:40324198_G_A | 65071-65106 | 65107-65140 | 65141-65176 |
| rs17466430_REF | | | |
| 12:40324198_G_A | 65080, 65086, | 65116, 65121, | 65150, 65152, |
| rs17466430_SNP | 65090, 65101, | 65124, 65126, | 65159, 65161, |
| | 65104, 65177- | 65136, 65204- | 65171, 65231- |
| | 65203 | 65230 | 65259 |
| 12:40324651_A_T | 65260-65288 | 65289-65300 | 65301-65316 |
| rs11176030_REF | | | |
| 12:40324651_A_T | 65264, 65268, | 65342-65353 | 65354-65369 |
| rs11176030_SNP | 65273, 65279, | | |
| | 65286, 65317- | | |
| | 65341 | | |
| 12:40325081_T_C | 65370-65377 | 65378-65387 | 65388-65399 |
| rs3747891_REF | | | |
| 12:40325081_T_C | 65371, 65373, | 65379-65380, | 65389, 65397, |
| rs3747891_SNP | 65400-65421 | 65422-65447 | 65448-65477 |
| 12:40325698_A_C | 65478-65509 | 65510-65537 | 65538-65569 |
| rs7298930_REF | | | |
| 12:40325698_A_C | 65480, 65483, | 65512, 65514, | 65542, 65544- |
| rs7298930_SNP | 65488, 65491, | 65516-65517, | 65545, 65552, |
| | 65493, 65496- | 65523, 65527- | 65556-65558, |
| | 65497, 65499- | 65528, 65530- | 65561-65562, |
| | 65501, 65570- | 65532, 65602- | 65566, 65630- |
| | 65601 | 65629 | 65665 |
| 12:40326004_G_C | 65666-65713 | 65714-65763 | 65764-65815 |
| rs17444089_REF | | | |
| 12:40326004_G_C | 65669, 65672, | 65717, 65720, | 65767, 65772, |
| rs17444089_SNP | 65674, 65680, | 65722, 65728, | 65778, 65780, |
| | 65682, 65687, | 65730, 65737, | 65787, 65800, |
| | 65689, 65692, | 65742, 65750, | 65802, 65808, |
| | 65694, 65702, | 65752, 65758, | 65810, 65812, |
| | 65816-65853 | 65854-65893 | 65894-65935 |
| 12:40326202_T_C | 65936 | 65937-65939 | 65940-65944 |
| rs10878377_REF | | | |
| 12:40326202_T_C | 65936 | 65937-65938, | 65940-65942, |
| rs10878377_SNP | | 65945 | 65946-65947 |
| 12:40326497_A_C | 65948-65959 | 65960-65971 | 65972-65983 |
| rs12316801_REF | | | |
| 12:40326497_A_C | 65952-65954, | 65965-65966, | 65976, 65978, |
| rs12316801_SNP | 65958-65959, | 65968, 65970- | 65980, 65982- |
| | 65984-65994 | 65971, 65995- | 65983, 66006- |
| | | 66005 | 66018 |
| 12:40326499_A_C | 65948-65959, | 65960-65971, | 65972-65983, |
| rs17444096_REF | 66019-66022 | 66023-66026 | 66027-66030 |
| 12:40326499_A_C | 65954, 65958- | 65968, 65970- | 65976, 65980, |
| rs17444096_SNP | 65959, 66021- | 65971, 66025- | 65983, 66029- |
| | 66022, 66031- | 66026, 66044- | 66030, 66057- |
| | 66043 | 66056 | 66069 |
| 12:40327240_G_A | 66070-66116 | 66117-66166 | 66167-66218 |
| rs11564175_REF | | | |
| 12:40327240_G_A | 66077, 66082, | 66125, 66130, | 66175, 66180, |
| rs11564175_SNP | 66084, 66099, | 66132, 66147, | 66182, 66189, |
| | 66101, 66104- | 66152-66154, | 66198, 66203, |
| | 66105, 66109, | 66158, 66164, | 66205-66206, |
| | 66112, 66116, | 66166, 66257- | 66210, 66216, |
| | 66219-66256 | 66296 | 66297-66338 |
| 12:40328650_A_G | 66339-66385 | 66386-66431 | 66432-66482 |
| rs2404832_REF | | | |
| 12:40328650_A_G | 66344, 66350, | 66398, 66400, | 66444, 66447, |
| rs2404832_SNP | 66353, 66369, | 66414, 66417, | 66458, 66462, |
| | 66371-66372, | 66419-66420, | 66467-66468, |
| | 66380-66381, | 66428, 66431, | 66474, 66477- |
| | 66383, 66385, | 66521-66560 | 66478, 66561- |
| | 66483-66520 | | 66602 |
| 12:40328747_C_A | 66603-66649 | 66650-66695 | 66696-66747 |
| rs17466486_REF | | | |
| 12:40328747_C_A | 66616, 66618- | 66664-66665, | 66701, 66713, |
| rs17466486_SNP | 66619, 66621, | 66667, 66677, | 66715-66716, |
| | 66624, 66631, | 66680-66681, | 66718, 66727, |
| | 66634-66635, | 66685, 66690, | 66730-66731, |
| | 66639, 66748- | 66783-66807 | 66736, 66741, |
| | 66782 | | 66808-66836 |
| 12:40328922_C_T | 66837-66883 | 66884-66931 | 66932-66981 |
| rs11564267_REF | | | |
| 12:40328922_C_T | 66841, 66844- | 66888, 66892, | 66936, 66939, |
| rs11564267_SNP | 66845, 66850, | 66897, 66900, | 66949, 66961, |
| | 66853, 66861, | 66914, 66922, | 66964, 66972, |
| | 66867, 66871, | 66928, 66931, | 66978, 66981, |
| | 66875, 66881, | 67020-67059 | 67060-67101 |
| | 66982-67019 | | |
| 12:40329067_AG_A | 67102-67126 | 67127-67132 | 67133-67146 |
| rs17520244_REF | | | |
| 12:40329067_AG_A | 67108-67109, | 67130 | 67135, 67139, |
| rs17520244_SNP | 67113, 67121, | | 67144 |
| | 67147-67148 | | |
| 12:40329673_T_A | 67149-67158 | 67159-67160 | 67161-67163 |
| rs10506152_REF | | | |
| 12:40329673_T_A | 67150, 67152, | 67159-67160 | 67161-67163 |
| rs10506152_SNP | 67155, 67157, | | |
| | 67164-67169 | | |
| 12:40330098_C_T | 67170-67212 | 67213-67255 | 67256-67300 |
| rs11176052_REF | | | |
| 12:40330098_C_T | 67173, 67183, | 67226, 67245, | 67266, 67270, |
| rs11176052_SNP | 67199, 67202, | 67254, 67339- | 67290, 67378- |
| | 67207, 67211, | 67377 | 67418 |
| | 67301-67338 | | |
| 12:40330228_C_T | 67419-67460 | 67461-67489 | 67490-67525 |
| rs11176053_REF | | | |
| 12:40330228_C_T | 67427, 67436, | 67463, 67469, | 67490, 67494, |
| rs11176053_SNP | 67440-67441, | 67477, 67481, | 67500, 67508, |
| | 67451, 67526- | 67544-67549 | 67512, 67550- |
| | 67543 | | 67559 |
| 12:40330692_A_C | 67560-67607 | 67608-67657 | 67658-67709 |
| rs17444124_REF | | | |
| 12:40330692_A_C | 67571, 67573, | 67619, 67621, | 67669, 67673- |
| rs17444124_SNP | 67575-67576, | 67623-67625, | 67676, 67701, |
| | 67594-67595, | 67644, 67650, | 67703-67706, |
| | 67597, 67601, | 67652-67654, | 67788-67829 |
| | 67603-67604, | 67748-67787 | |
| | 67710-67747 | | |
| 12:40330779_G_A | 67830-67877 | 67878-67925 | 67926-67976 |
| rs11564203_REF | | | |
| 12:40330779_G_A | 67832-67833, | 67879, 67881, | 67927, 67929, |
| rs11564203_SNP | 67837, 67840, | 67885, 67896, | 67933, 67948- |
| | 67849, 67852, | 67899, 67901, | 67949, 67951, |
| | 67854, 67856, | 67903, 67920, | 67953, 67955, |
| | 67860, 67875, | 68014-68042 | 67971, 68043- |
| | 67977-68013 | | 68077 |
| 12:40331158_G_T | 68078-68123 | 68124-68167 | 68168-68215 |
| rs7307562_REF | | | |
| 12:40331158_G_T | 68078, 68089- | 68124-68125, | 68168, 68170, |
| rs7307562_SNP | 68091, 68099, | 68136, 68159, | 68181-68182, |
| | 68111, 68115, | 68164, 68253- | 68184, 68212, |
| | 68120, 68216- | 68281 | 68282-68316 |
| | 68252 | | |
| 12:40331402_T_A | 68317-68364 | 68365-68414 | 68415-68466 |
| rs17520251_REF | | | |
| 12:40331402_T_A | 68317-68320, | 68365-68368, | 68415-68418, |
| rs17520251_SNP | 68324, 68326, | 68372, 68374, | 68430, 68436, |
| | 68332, 68334, | 68380, 68386, | 68443, 68447, |
| | 68344, 68350, | 68393, 68399, | 68450, 68454, |
| | 68467-68504 | 68505-68544 | 68545-68586 |
| 12:40331597_C_CA | 68587 | , | 68588 |
| rs11461395_REF | | | |
| 12:40331769_T_C | 68589-68616 | 68617-68642 | 68643-68670 |
| rs10506153_REF | | | |
| 12:40331769_T_C | 68594, 68598, | 68622, 68625, | 68648, 68651- |
| rs10506153_SNP | 68600, 68605, | 68632, 68636, | 68652, 68659, |
| | 68611, 68671- | 68638, 68696- | 68663, 68721- |
| | 68695 | 68720 | 68747 |
| 12:40331791_C_G | 68748-68758 | | |
| rs73275761_REF | | | |
| 12:40331791_C_G | 68759-68770 | | |
| rs73275761_SNP | | | |
| 12:40331976_G_A | 68771-68818 | 68819-68868 | 68869-68920 |
| rs963243_REF | | | |
| 12:40331976_G_A | 68778-68779, | 68826, 68833- | 68874, 68877- |
| rs963243_SNP | 68785, 68793, | 68834, 68842- | 68878, 68885- |
| | 68799, 68804- | 68843, 68849, | 68886, 68894, |
| | 68805, 68808- | 68854-68855, | 68901, 68906- |
| | 68809, 68811, | 68858-68859, | 68907, 68910, |
| | 68921-68958 | 68959-68994 | 68995-69034 |
| 12:40332078_C_T | 69035-69080 | 69081-69130 | 69131-69182 |
| rs17444145_REF | | | |
| 12:40332078_C_T | 69045-69046, | 69091-69092, | 69135, 69142- |
| rs17444145_SNP | 69057, 69065, | 69094, 69099, | 69143, 69145, |
| | 69067, 69069, | 69105, 69114, | 69150, 69161, |
| | 69072-69073, | 69116, 69118, | 69166, 69170, |
| | 69077-69078, | 69122, 69127, | 69174, 69179, |
| | 69183-69220 | 69221-69260 | 69261-69302 |
| 12:40332126_A_G | 69303-69348 | 69349-69398 | 69399-69450 |
| rs10878386_REF | | | |
| 12:40332126_A_G | 69319, 69332- | 69362, 69381- | 69412, 69431, |
| rs10878386_SNP | 69333, 69335, | 69382, 69384, | 69434-69435, |
| | 69337, 69339, | 69388, 69391, | 69439, 69443, |
| | 69342, 69344- | 69393-69395, | 69445-69447, |
| | 69346, 69451- | 69397, 69489- | 69449, 69529- |
| | 69488 | 69528 | 69570 |
| 12:40332765_C_G | 69571-69572 | | |
| rs17466521_REF | | | |
| 12:40332765_C_G | 69571-69572 | | |
| rs17466521_SNP | | | |
| 12:40332845_A_T | 69573-69604 | 69605-69630 | 69631-69658 |
| rs12367542_REF | | | |
| 12:40332845_A_T | 69579, 69590- | 69609, 69611, | 69636, 69638- |
| rs12367542_SNP | 69591, 69599- | 69620, 69626- | 69639, 69648, |
| | 69600, 69659- | 69627, 69686- | 69654, 69707- |
| | 69685 | 69706 | 69729 |
| 12:40332946_T_G | 69730-69768 | 69769-69798 | 69799-69828 |
| rs17520278_REF | | | |
| 12:40332946_T_G | 69768, 69829- | 69867-69905 | 69906-69943 |
| rs17520278_SNP | 69866 | | |
| 12:40332960_T_TAA | 69738, 69745- | 69784, 69790, | 69814, 69819, |
| rs35031086_REF | 69746, 69748, | 69793-69798 | 69821-69828 |
| | 69754, 69758- | | |
| | 69768, 69944- | | |
| | 69945 | | |
| 12:40332960_T_TA | 69738, 69745- | 69784, 69790, | 69814, 69819, |
| rs35031086_REF | 69746, 69748, | 69793-69798 | 69821-69828 |
| | 69754, 69758- | | |
| | 69768, 69944- | | |
| | 69945 | | |
| 12:40333796_C_T | 69946-69992 | 69993-70042 | 70043-70094 |
| rs11834950_REF | | | |
| 12:40333796_C_T | 69947-69948, | 69993, 69995- | 70043, 70046, |
| rs11834950_SNP | 69954-69956, | 69996, 70002, | 70052, 70054- |
| | 69965, 69967, | 70004-70005, | 70055, 70073, |
| | 69976, 69982, | 70014, 70025, | 70076-70077, |
| | 69986, 70095- | 70031, 70035, | 70083, 70087, |
| | 70132 | 70133-70172 | 70173-70214 |
| 12:40333977_T_G | 70215-70262 | 70263-70312 | 70313-70364 |
| rs11829088_REF | | | |
| 12:40333977_T_G | 70222, 70229, | 70267, 70271, | 70313, 70318, |
| rs11829088_SNP | 70232-70233, | 70278, 70281- | 70322, 70330, |
| | 70235, 70239, | 70282, 70284, | 70333-70334, |
| | 70246, 70249, | 70288, 70295, | 70336, 70347, |
| | 70259, 70261, | 70298, 70308, | 70350, 70360, |
| | 70365-70402 | 70403-70442 | 70443-70484 |
| 12:40334389_C_A | 70485-70520 | 70521-70553 | 70554-70591 |
| rs4465428_REF | | | |
| 12:40334389_C_A | 70502-70505, | 70538-70540, | 70568, 70573- |
| rs4465428_SNP | 70508, 70515, | 70542, 70548, | 70575, 70577, |
| | 70592-70610 | 70611-70630 | 70585, 70631- |
| | | | 70655 |
| 12:40334496_A_G | 70656-70659 | | |
| rs61007767_REF | | | |
| 12:40334496_A_G | 70656-70664 | | 70665-70667 |
| rs61007767_SNP | | | |
| 12:40334626_G_C | 70668-70680 | 70681-70697 | 70698-70718 |
| rs60776971_REF | | | |
| 12:40334626_G_C | 70671, 70719- | 70681, 70686, | 70699, 70705- |
| rs60776971_SNP | 70760 | 70761-70801 | 70706, 70802- |
| | | | 70846 |
| 12:40335205_C_T | 70847-70893 | 70894-70943 | 70944-70995 |
| rs2404834_REF | | | |
| 12:40335205_C_T | 70856, 70860, | 70907, 70911, | 70957, 70965- |
| rs2404834_SNP | 70862-70863, | 70915, 70929- | 70966, 70980- |
| | 70867, 70881- | 70932, 70934, | 70981, 70983, |
| | 70882, 70884, | 70942-70943, | 70985, 70993- |
| | 70892-70893, | 71034-71073 | 70995, 71074- |
| | 70996-71033 | | 71115 |
| 12:40335853_G_A | 71116-71163 | 71164-71213 | 71214-71265 |
| rs7302841_REF | | | |
| 12:40335853_G_A | 71118, 71126, | 71167, 71175- | 71217, 71225- |
| rs7302841_SNP | 71140, 71144, | 71176, 71190, | 71226, 71231, |
| | 71148, 71153, | 71194, 71198, | 71244, 71246, |
| | 71156, 71159, | 71203, 71209, | 71250, 71261, |
| | 71161, 71163, | 71211, 71213, | 71263, 71265, |
| | 71266-71303 | 71304-71343 | 71344-71385 |
| 12:40336661_C_T | 71386-71433 | 71434-71482 | 71483-71534 |
| rs2404835_REF | | | |
| 12:40336661_C_T | 71393, 71395, | 71443, 71453, | 71491, 71493, |
| rs2404835_SNP | 71415-71416, | 71462, 71465- | 71501, 71504, |
| | 71418, 71426, | 71466, 71468, | 71513, 71516, |
| | 71429-71430, | 71476, 71480, | 71519, 71527, |
| | 71432-71433, | 71482, 71573- | 71531, 71534, |
| | 71535-71572 | 71610 | 71611-71652 |
| 12:40337133_CA_C | 71653-71700 | 71701-71746 | 71747-71798 |
| rs11356084_REF | | | |
| 12:40337133_CA_C | 71654, 71660, | 71708, 71711- | 71750, 71756, |
| rs11356084_SNP | 71663, 71668, | 71712, 71715, | 71760-71761, |
| | 71675, 71681, | 71722, 71725, | 71766, 71773, |
| | 71684, 71688, | 71731, 71734, | 71776, 71786, |
| | 71690, 71799- | 71736, 71835- | 71788, 71873- |
| | 71834 | 71872 | 71912 |
| 12:40337381_A_C | 71913-71916 | 71917-71919 | 71920-71924 |
| rs715403_REF | | | |
| 12:40337381_A_C | 71913-71916, | 71917-71919, | 71920-71924, |
| rs715403_SNP | 71925-71929 | 71930 | 71931-71933 |
| 12:40337412_G_A | 71934-71978 | 71979-72010 | 72011-72050 |
| rs715402_REF | | | |
| 12:40337412_G_A | 71934, 71937, | 71980, 71989, | 72012, 72015, |
| rs715402_SNP | 71941, 71949, | 71998, 72070- | 72035, 72038, |
| | 71952, 71965, | 72076 | 72045, 72077- |
| | 71969, 72051- | | 72091 |
| | 72069 | | |
| 12:40337704_G_C | 72092-72123 | 72124-72149 | 72150-72177 |
| rs6581667_REF | | | |
| 12:40337704_G_C | 72095, 72100, | 72127, 72134, | 72154, 72158, |
| rs6581667_SNP | 72106, 72108, | 72136, 72143, | 72164, 72171, |
| | 72116, 72178- | 72148, 72207- | 72176, 72228- |
| | 72206 | 72227 | 72250 |
| 12:40337804_G_A | 72251-72298 | 72299-72342 | 72343-72389 |
| rs6581668_REF | | | |
| 12:40337804_G_A | 72253, 72272, | 72301, 72308, | 72345, 72354, |
| rs6581668_SNP | 72274, 72280, | 72323, 72325, | 72369, 72371, |
| | 72282, 72284, | 72327-72328, | 72373-72374, |
| | 72287, 72291, | 72335, 72337- | 72376, 72382, |
| | 72293-72294, | 72338, 72428- | 72384-72385, |
| | 72390-72427 | 72467 | 72468-72509 |
| 12:40338157_CCCT_C | 72510-72556 | 72557-72606 | 72607-72658 |
| rs200521371_REF | | | |
| 12:40338157_CCCT_C | 72520, 72522, | 72559, 72571, | 72609, 72627- |
| rs200521371_SNP | 72529, 72538, | 72578, 72588, | 72628, 72638, |
| | 72547, 72551, | 72597, 72599, | 72642, 72648, |
| | 72553, 72659- | 72603, 72697- | 72651, 72737- |
| | 72696 | 72736 | 72778 |
| 12:40338159_C_T | 72510-72527, | 72557-72576, | 72607-72632, |
| rs4767970_REF | 72529-72534, | 72578-72582, | 72634, 72636, |
| | 72536, 72538- | 72584, 72586, | 72638-72649, |
| | 72548, 72550- | 72588-72606, | 72651-72658, |
| | 72556, 72661, | 72720, 72724, | 72745, 72762, |
| | 72685, 72779- | 72781-72782 | 72783-72784 |
| | 72780 | | |
| 12:40338159_C_T | 72513, 72520, | 72559, 72568, | 72609, 72621, |
| rs4767970_SNP | 72522, 72529, | 72571, 72578, | 72627-72628, |
| | 72538, 72547, | 72588, 72597, | 72638, 72642, |
| | 72551, 72553, | 72599, 72603, | 72648, 72651, |
| | 72661, 72685- | 72720, 72724, | 72745, 72762, |
| | 72686, 72785- | 72732, 72822- | 72775, 72861- |
| | 72821 | 72860 | 72901 |
| 12:40338230_C_T | 72902-72949 | 72950-72999 | 73000-73051 |
| rs4767971_REF | | | |
| 12:40338230_C_T | 72902-72903, | 72950, 72953- | 73004, 73013, |
| rs4767971_SNP | 72905-72906, | 72954, 72963, | 73015-73016, |
| | 72914-72915, | 72965-72966, | 73028, 73033, |
| | 72917-72918, | 72978, 72986- | 73037-73038, |
| | 72937, 72946, | 72987, 72996, | 73043, 73048, |
| | 73052-73089 | 73090-73129 | 73130-73171 |
| 12:40338321_C_T | 73172-73209 | 73210-73235 | 73236-73265 |
| rs4767972_REF | | | |
| 12:40338321_C_T | 73172-73173, | 73210-73211, | 73236-73238, |
| rs4767972_SNP | 73182, 73198, | 73215, 73219, | 73248, 73258, |
| | 73205, 73266- | 73229, 73283- | 73300-73318 |
| | 73282 | 73299 | |
| 12:40338592_T_C | 73319-73341 | 73342-73345 | 73346-73351 |
| rs1427271_REF | | | |
| 12:40338592_T_C | 73352-73384 | 73385-73412 | 73413-73446 |
| rs1427271_SNP | | | |
| 12:40338678_G_C | 73447-73487 | 73488-73526 | 73527-73567 |
| rs1427272_REF | | | |
| 12:40338678_G_C | 73452, 73460, | 73606-73644 | 73645-73685 |
| rs1427272_SNP | 73464, 73568- | | |
| | 73605 | | |
| 12:40338928_C_G | 73686-73733 | 73734-73779 | 73780-73828 |
| rs1427273_REF | | | |
| 12:40338928_C_G | 73686, 73690- | 73737, 73745, | 73783, 73786, |
| rs1427273_SNP | 73691, 73699, | 73754, 73760, | 73792, 73802, |
| | 73701, 73711, | 73764, 73769, | 73808-73809, |
| | 73716, 73718, | 73867-73906 | 73813, 73907- |
| | 73723, 73733, | | 73948 |
| | 73829-73866 | | |
| 12:40339098 C A | 73949-73993 | 73994-74041 | 74042-74091 |
| rs10735934 REF | | | |
| 12:40339098_C_A | 73949, 73951, | 73994, 73996, | 74042, 74044, |
| rs10735934_SNP | 73954, 73959, | 73999, 74004- | 74047, 74053, |
| | 73968, 73972, | 74005, 74014, | 74062, 74071, |
| | 73980-73981, | 74016, 74028, | 74076, 74078, |
| | 74092-74128 | 74129-74165 | 74166-74207 |
| 12:40339280_A_G | 74208-74225 | 74226-74241 | 74242-74261 |
| rs10732751_REF | | | |
| 12:40339280_A_G | 74213, 74218, | 74229, 74234, | 74247, 74252, |
| rs10732751_SNP | 74221, 74224- | 74237, 74240- | 74255, 74259, |
| | 74225, 74262- | 74241, 74285- | 74261, 74300- |
| | 74284 | 74299 | 74318 |
| 12:40339762_T_G | 74319-74337 | 74338-74348 | 74349-74363 |
| rs12306060_REF | | | |
| 12:40339762_T_G | 74319-74320, | 74338, 74343, | 74354-74355, |
| rs12306060_SNP | 74323, 74328, | 74383-74399 | 74357, 74359, |
| | 74364-74382 | | 74400-74420 |
| 12:40340781_T_C | 74421-74465 | 74466-74505 | 74506-74548 |
| rs1365763_REF | | | |
| 12:40340781_T_C | 74427, 74429, | 74468, 74470, | 74506, 74510, |
| rs1365763_SNP | 74434-74435, | 74475, 74479, | 74512, 74517, |
| | 74440, 74446- | 74485-74486, | 74521, 74527, |
| | 74447, 74459, | 74498, 74502, | 74540, 74544, |
| | 74463, 74465, | 74505, 74587- | 74547, 74625- |
| | 74549-74586 | 74624 | 74666 |
| 12:40341114_C_A | 74667-74714 | 74715-74764 | 74765-74816 |
| rs7963697_REF | | | |
| 12:40341114_C_A | 74667-74668, | 74715-74716, | 74765, 74786- |
| rs7963697_SNP | 74670, 74674, | 74736-74737, | 74788, 74790, |
| | 74688, 74690, | 74739, 74741, | 74801, 74807, |
| | 74692, 74706, | 74750, 74756, | 74809-74810, |
| | 74708-74709, | 74758-74759, | 74814, 74895- |
| | 74817-74854 | 74855-74894 | 74936 |
| 12:40341174_TG_T | 74937-74983 | 74984-75033 | 75034-75085 |
| rs11316380_REF | | | |
| 12:40341174_TG_T | 74940, 74946, | 74988, 74995, | 75045, 75056, |
| rs11316380_SNP | 74948, 74962- | 75006, 75012, | 75064, 75066, |
| | 74963, 74965, | 75014, 75017, | 75068, 75073, |
| | 74968, 74978, | 75022, 75028, | 75079, 75081, |
| | 74981, 75086- | 75031, 75122- | 75083, 75160- |
| | 75121 | 75159 | 75199 |
| 12:40341367_G_C | 75200-75245 | 75246-75291 | 75292-75340 |
| rs7963086_REF | | | |
| 12:40341367_G_C | 75200, 75202, | 75246, 75248, | 75292, 75294, |
| rs7963086_SNP | 75204, 75208, | 75250, 75254, | 75296, 75305, |
| | 75213, 75218, | 75266, 75282, | 75313, 75317- |
| | 75224, 75236, | 75379-75418 | 75318, 75419- |
| | 75341-75378 | | 75460 |
| 12:40341862_A_G | 75461-75466 | 75467-75471 | 75472-75477 |
| rs17491536_REF | | | |
| 12:40341862_A_G | 75461, 75463- | 75467-75471, | 75472-75473, |
| rs17491536_SNP | 75466, 75478- | 75487-75489 | 75475-75477, |
| | 75486 | | 75490-75494 |
| 12:40341962_T_C | 75495-75541 | 75542-75585 | 75586-75634 |
| rs7956787_REF | | | |
| 12:40341962_T_C | 75495-75496, | 75548, 75553, | 75593, 75599, |
| rs7956787_SNP | 75504-75505, | 75557, 75559, | 75603, 75605, |
| | 75510, 75514, | 75564, 75576- | 75610, 75616, |
| | 75516, 75521, | 75577, 75673- | 75623-75624, |
| | 75534, 75538, | 75712 | 75713-75754 |
| | 75635-75672 | | |
| 12:40342002_T_G | 75755-75802 | 75803-75846 | 75847-75898 |
| rs7956898_REF | | | |
| 12:40342002_T_G | 75759, 75765, | 75806, 75809, | 75857, 75865, |
| rs7956898_SNP | 75767, 75769, | 75817-75818, | 75867, 75871, |
| | 75773, 75777, | 75822, 75828, | 75877, 75879, |
| | 75783, 75785, | 75830, 75832, | 75881, 75883, |
| | 75787, 75793, | 75838, 75841, | 75888, 75892, |
| | 75899-75936 | 75937-75976 | 75977-76018 |
| 12:40342137_G_A | 76019-76066 | 76067-76116 | 76117-76168 |
| rs10506155_REF | | | |
| 12:40342137_G_A | 76019, 76021, | 76069, 76075, | 76119, 76125, |
| rs10506155_SNP | 76027, 76031, | 76078, 76080, | 76128, 76130- |
| | 76041, 76049- | 76090, 76099, | 76131, 76153, |
| | 76050, 76053, | 76102, 76107, | 76158, 76163, |
| | 76058, 76064, | 76112, 76114, | 76165, 76168, |
| | 76169-76206 | 76207-76246 | 76247-76288 |
| 12:40342208_A_G | 76289-76336 | 76337-76386 | 76387-76438 |
| rs7954061_REF | | | |
| 12:40342208_A_G | 76289-76290, | 76337-76338, | 76387-76388, |
| rs7954061_SNP | 76295, 76300- | 76348-76349, | 76390, 76399- |
| | 76301, 76304, | 76352, 76358, | 76400, 76403, |
| | 76315, 76324, | 76361, 76374, | 76409, 76412, |
| | 76326, 76330, | 76376, 76380, | 76427, 76430, |
| | 76439-76476 | 76477-76516 | 76517-76558 |
| 12:40342241_T_A | 76559-76606 | 76607-76656 | 76657-76708 |
| rs7957151_REF | | | |
| 12:40342241_T_A | 76559, 76561, | 76607, 76609, | 76657, 76659, |
| rs7957151_SNP | 76564, 76566- | 76612, 76614- | 76664, 76673, |
| | 76567, 76572, | 76615, 76623, | 76679-76680, |
| | 76575, 76584, | 76632, 76637, | 76684, 76689, |
| | 76589-76590, | 76646, 76649, | 76698, 76701, |
| | 76709-76746 | 76747-76786 | 76787-76828 |
| 12:40342447_C_T | 76829-76876 | 76877-76925 | 76926-76977 |
| rs919174_REF | | | |
| 12:40342447_C_T | 76831-76832, | 76879, 76881, | 76926, 76929, |
| rs919174_SNP | 76838, 76842, | 76887, 76891, | 76937, 76941, |
| | 76845-76846, | 76895, 76905, | 76945, 76955, |
| | 76856, 76859, | 76908-76909, | 76958-76960, |
| | 76862-76863, | 76912, 77016- | 76964, 77051- |
| | 76978-77015 | 77050 | 77089 |
| 12:40342479_C_CTT | 77090-77101 | 77102-77113 | 77114-77127 |
| rs3065921_REF | | | |
| 12:40342479_C_CT | 77090-77101 | 77102-77113 | 77114-77127 |
| rs3065921_REF | | | |
| 12:40342603_C_T | 77128-77165 | 77166-77201 | 77202-77239 |
| rs919175_REF | | | |
| 12:40342603_C_T | 77138, 77153- | 77176-77177, | 77212-77213, |
| rs919175_SNP | 77154, 77161- | 77191-77192, | 77228, 77235- |
| | 77162, 77240- | 77198, 77269- | 77236, 77298- |
| | 77268 | 77297 | 77328 |
| 12:40342980_C_T | 77329-77348 | 77349-77366 | 77367-77386 |
| rs1035812_REF | | | |
| 12:40342980_C_T | 77333, 77338, | 77357, 77359, | 77374, 77377, |
| rs1035812_SNP | 77341, 77346, | 77364-77366, | 77379, 77385- |
| | 77348, 77387- | 77398-77408 | 77386, 77409- |
| | 77397 | | 77421 |
| 12:40343200_G_A | 77422-77441 | 77442-77455 | 77456-77472 |
| rs1365764_REF | | | |
| 12:40343200_G_A | 77426, 77428, | 77444-77445, | 77459-77462, |
| rs1365764_SNP | 77431, 77437, | 77447, 77452, | 77464, 77469, |
| | 77440, 77473- | 77454, 77480- | 77485-77491 |
| | 77479 | 77484 | |
| 12:40343257_C_CTCAG | 77492-77538 | 77539-77585 | 77586-77635 |
| rs397724721_REF | | | |
| 12:40343257_C_CTCAG | 77492, 77503, | 77548, 77550, | 77596, 77598- |
| rs397724721_SNP | 77519, 77522, | 77567, 77570- | 77599, 77613, |
| | 77536, 77538, | 77571, 77585, | 77615, 77619, |
| | 77636-77660 | 77661-77687 | 77688-77716 |
| 12:40343273_C_T | 77492-77494, | 77539-77541, | 77586-77588, |
| rs17461964_REF | 77496-77498, | 77543-77545, | 77591-77593, |
| | 77504, 77507- | 77551, 77555- | 77599, 77603- |
| | 77508, 77511, | 77557, 77559, | 77605, 77607, |
| | 77523-77524, | 77571-77572, | 77616, 77620- |
| | 77526, 77534- | 77581-77582, | 77621, 77623, |
| | 77535, 77717- | 77732-77740 | 77625, 77631- |
| | 77731 | | 77632, 77741- |
| | | | 77752 |
| 12:40343273_C_T | 77493, 77497, | 77540, 77544, | 77587, 77592, |
| rs17461964_SNP | 77508, 77511, | 77556-77557, | 77605, 77607, |
| | 77535, 77721, | 77559, 77732, | 77625, 77742- |
| | 77723-77724, | 77735-77736, | 77743, 77747, |
| | 77726, 77731, | 77738, 77740, | 77749, 77752, |
| | 77753-77763 | 77764-77772 | 77773-77784 |
| 12:40343313_G_T | 77785-77830 | 77831-77880 | 77881-77932 |
| rs10784518_REF | | | |
| 12:40343313_G_T | 77785-77786, | 77831-77832, | 77881, 77889, |
| rs10784518_SNP | 77798, 77804, | 77845, 77851- | 77896, 77902- |
| | 77811, 77814, | 77852, 77859, | 77904, 77911, |
| | 77821, 77825- | 77864, 77870, | 77916, 77922, |
| | 77826, 77933- | 77875-77876, | 77927, 78011- |
| | 77970 | 77971-78010 | 78052 |
| 12:40343381_G_A | 78053-78073 | 78074-78082 | 78083-78097 |
| rs4768231_REF | | | |
| 12:40343381_G_A | 78056, 78058, | 78105-78110 | 78090, 78111- |
| rs4768231_SNP | 78069-78070, | | 78118 |
| | 78098-78104 | | |
| 12:40343438_C_T | 78119-78164 | 78165-78211 | 78212-78262 |
| rs4768232_REF | | | |
| 12:40343438_C_T | 78125, 78128, | 78171, 78173, | 78218, 78220, |
| rs4768232_SNP | 78133, 78140- | 78176, 78181, | 78223, 78228, |
| | 78141, 78148, | 78189, 78196, | 78230, 78237, |
| | 78155, 78158- | 78203, 78299- | 78245, 78250, |
| | 78159, 78263- | 78333 | 78253, 78334- |
| | 78298 | | 78374 |
| 12:40344001_A_G | 78375-78402 | 78403-78408 | 78409-78418 |
| rs3943893_REF | | | |
| 12:40344001_A_G | 78419-78455 | 78456-78487 | 78488-78526 |
| rs3943893_SNP | | | |
| 12:40344030_T_C | 78527-78536 | | |
| rs2162471_REF | | | |
| 12:40344030_T_C | 78530, 78533, | 78560-78570 | 78571-78587 |
| rs2162471_SNP | 78537-78559 | | |
| 12:40344106_C_T | 78588-78609 | 78610-78631 | 78632-78653 |
| rs1035813_REF | | | |
| 12:40344106_C_T | 78593, 78596, | 78618-78619, | 78640, 78643, |
| rs1035813_SNP | 78601-78602, | 78623-78624, | 78645-78646, |
| | 78604, 78606, | 78626, 78628, | 78650-78651, |
| | 78608, 78654- | 78630, 78669- | 78653, 78682- |
| | 78668 | 78681 | 78696 |
| 12:40344206_C_T | 78697-78731 | 78732-78760 | 78761-78796 |
| rs10459264_REF | | | |
| 12:40344206_C_T | 78697-78699, | 78732-78733, | 78761, 78770, |
| rs10459264_SNP | 78703, 78712- | 78741, 78744, | 78773-78774, |
| | 78713, 78718- | | |
| | 78719, 78797- | 78747, 78751, | 78778-78779, |
| | 78817 | 78818-78838 | 78784-78785, |
| | | | 78839-78861 |
| 12:40344501_G_A | 78862-78909 | 78910-78959 | 78960-79011 |
| rs10459265_REF | | | |
| 12:40344501_G_A | 78878, 78880, | 78916, 78925, | 78961, 78964, |
| rs10459265_SNP | 78885-78886, | 78928, 78935- | 78968, 78977, |
| | 78894-78895, | 78936, 78944- | 78980, 78988, |
| | 78897-78898, | 78945, 78948, | 78996, 79000, |
| | 78900, 78905, | 78950, 78955, | 79002, 79007, |
| | 79012-79049 | 79050-79085 | 79086-79127 |
| 12:40345691_G_A | 79128-79165 | 79166-79202 | 79203-79241 |
| rs11564147_REF | | | |
| 12:40345691_G_A | 79131, 79140, | 79169, 79178, | 79206, 79209, |
| rs11564147_SNP | 79145, 79153, | 79183, 79190, | 79221, 79229, |
| | 79164, 79242- | 79273-79303 | 79304-79336 |
| | 79272 | | |
| 12:40345826_TA_T | 79337-79340 | | |
| rs10715759_REF | | | |
| 12:40345826_TA_T | 79337, 79339, | | |
| rs10715759_SNP | 79341-79346 | | |
| 12:40345925_C_T | 79347-79385 | 79386-79422 | 79423-79463 |
| rs7296657_REF | | | |
| 12:40345925_C_T | 79368, 79380, | 79417, 79421, | 79461, 79463, |
| rs7296657_SNP | 79384, 79464- | 79497-79519 | 79520-79548 |
| | 79496 | | |
| 12:40346050_A_T | 79549-79562 | 79563-79576 | 79577-79592 |
| rs7312497_REF | | | |
| 12:40346050_A_T | 79554, 79556, | 79569, 79571, | 79581, 79584, |
| rs7312497_SNP | 79558, 79560, | 79573, 79575- | 79589, 79591- |
| | 79562, 79593- | 79576, 79602- | 79592, 79611- |
| | 79601 | 79610 | 79621 |
| 12:40346187_CT_C | 79622-79639 | 79640-79657 | 79658-79675 |
| rs10715758_REF | | | |
| 12:40346187_CT_C | 79634-79635 | 79646, 79652 | 79662, 79664 |
| rs10715758_SNP | | | |
| 12:40346563_G_T | 79676-79723 | 79724-79773 | 79774-79825 |
| rs10784522_REF | | | |
| 12:40346563_G_T | 79683, 79692, | 79731, 79740, | 79781, 79790, |
| rs10784522_SNP | 79697, 79703- | 79745, 79751, | 79795, 79801, |
| | 79704, 79708, | 79757, 79761, | 79807, 79811, |
| | 79712, 79718- | 79767-79768, | 79817, 79822, |
| | 79719, 79723, | 79771, 79773, | 79824-79825, |
| | 79826-79863 | 79864-79903 | 79904-79945 |
| 12:40347026_AT_A | 79946-79954 | 79955 | 79956-79957 |
| rs796827140_REF | | | |
| 12:40347026_AT_A | 79950 | | |
| rs796827140_SNP | | | |
| 12:40347033_T_G | 79950, 79953 | | |
| rs74467833_REF | | | |
| 12:40347033_T_G | 79950, 79953, | | 79966-79967 |
| rs74467833_SNP | 79958-79965 | | |
| 12:40347145_A_G | 79968-79970 | 79971-79973 | 79974-79977 |
| rs7131694_REF | | | |
| 12:40347145_A_G | 79968-79970 | 79971-79973 | 79974-79977 |
| rs7131694_SNP | | | |
| 12:40347815_T_C | 79978-79979 | 79980-79983 | 79984-79989 |
| rs12311273_REF | | | |
| 12:40347815_T_C | 79978, 79990- | 79980, 79982, | 79985, 79987- |
| rs12311273_SNP | 79991 | 79992-79994 | 79988, 79995- |
| | | | 80000 |
| 12:40347876_T_C | 80001-80005 | 80006-80014 | 80015-80027 |
| rs58911468_REF | | | |
| 12:40347876_T_C | 80001-80003, | 80007-80009, | 80016, 80020- |
| rs58911468_SNP | 80028-80042 | 80013, 80043- | 80021, 80023, |
| | | 80060 | 80026, 80061- |
| | | | 80081 |
| 12:40347956_T_TAA | 80082-80084 | 80085-80089 | 80090-80096 |
| rs35847030_REF | | | |
| 12:40347956_T_TA | 80082-80084 | 80085-80089 | 80090-80096 |
| rs35847030_REF | | | |
| 12:40348019_A_G | 80097-80124 | 80125-80148 | 80149-80178 |
| rs1365765_REF | | | |
| 12:40348019_A_G | 80101-80103, | 80129-80131, | 80155-80157, |
| rs1365765_SNP | 80105-80107, | 80134, 80136- | 80160, 80162, |
| | 80109-80110, | 80137, 80139- | 80165, 80168- |
| | 80118, 80120, | 80140, 80145- | 80169, 80174- |
| | 80179-80210 | 80146, 80211- | 80175, 80235- |
| | | 80234 | 80268 |
| 12:40348202_AT_A | 80269-80278 | 80279-80287 | 80288-80299 |
| rs66513412_REF | | | |
| 12:40348202_AT_A | 80270, 80277- | 80280, 80287, | 80289-80290, |
| rs66513412_SNP | 80278, 80300- | 80306-80311 | 80293, 80312- |
| | 80305 | | 80319 |
| 12:40348203_T_C | 80270-80272, | 80280, 80282- | 80289-80293, |
| rs2896977_REF | 80274-80278 | 80287 | 80295-80299 |
| 12:40348203_T_C | 80270, 80277- | 80280, 80287, | 80289-80290, |
| rs2896977_SNP | 80278, 80320- | 80327-80333 | 80293, 80334- |
| | 80326 | | 80342 |
| 12:40348204_A_C | 80270-80272, | 80280, 80282, | 80289, 80291, |
| rs199606989_REF | 80275-80278, | 80284-80287 | 80293, 80295- |
| | 80343 | | 80299 |
| 12:40348204_A_C | 80270, 80275, | 80280, 80286- | 80289, 80293, |
| rs_199606989_SNP | 80277-80278, | 80287, 80349- | 80299, 80354- |
| | 80343-80348 | 80353 | 80360 |
| 12:40348271_C_T | 80361-80364 | , | , |
| rs1365766_REF | | | |
| 12:40348452_G_A | 80365-80384 | 80385-80406 | 80407-80430 |
| rs10878405_REF | | | |
| 12:40348452_G_A | 80374, 80377, | 80396-80397, | 80419-80421, |
| rs10878405_SNP | 80379, 80381, | 80400, 80402, | 80426, 80429, |
| | 80383, 80431- | 80405, 80446- | 80461-80477 |
| | 80445 | 80460 | |
| 12:40348561_G_A | 80478-80488 | 80489-80492 | 80493-80500 |
| rs11176143_REF | | | |
| 12:40348561_G_A | 80479, 80482, | 80491-80492 | 80497, 80499- |
| rs11176143_SNP | 80486-80487 | | 80500 |
| 12:40348887_T_C | 80501-80531 | 80532-80562 | 80563-80600 |
| rs7294958_REF | | | |
| 12:40348887_T_C | 80505, 80509- | 80537, 80540- | 80567, 80569, |
| rs7294958_SNP | 80510, 80512, | 80541, 80543, | 80573-80574, |
| | 80518-80519, | 80546, 80549- | 80577, 80580, |
| | 80522, 80524- | 80550, 80554, | 80585, 80590, |
| | 80525, 80528, | 80556, 80559, | 80592, 80596, |
| | 80601-80634 | 80635-80666 | 80667-80704 |
| 12:40348899_C_T | 80501-80506, | 80532-80542, | 80563-80566, |
| rs61915610_REF | 80508-80511, | 80544, 80551- | 80568-80570, |
| | 80513, 80520- | 80555, 80557- | 80572-80574, |
| | 80523, 80526- | 80562, 80724- | 80576, 80578, |
| | 80531, 80705- | 80741 | 80581, 80587- |
| | 80723 | | 80591, 80593- |
| | | | 80600, 80742- |
| | | | 80759 |
| 12:40348899_C_T | 80520, 80526, | 80557, 80561, | 80581, 80598, |
| rs61915610_SNP | 80530, 80760- | 80797-80833 | 80600, 80834- |
| | 80796 | | 80872 |
| 12:40349074_A_T | 80873-80890 | 80891-80892 | 80893-80898 |
| rs7294952_REF | | | |
| 12:40349074_A_T | 80873, 80878, | 80912-80913 | 80914-80919 |
| rs7294952_SNP | 80882, 80887, | | |
| | 80890, 80899- | | |
| | 80911 | | |
| 12:40349558_GGCTGGAGT_G | | | 80920 |
| rs59096461_REF | | | |
| 12:40349690_A_AT | 80921-80926 | 80927-80933 | 80934-80943 |
| rs59980086_REF | | | |
| 12:40349986_C_T | 80944-80991 | 80992-81037 | 81038-81086 |
| rs4768233_REF | | | |
| 12:40349986_C_T | 80946-80947, | 81005-81006, | 81041, 81052- |
| rs4768233_SNP | 80955, 80959, | 81019, 81026, | 81053, 81065, |
| | 80972, 80975, | 81031, 81034, | 81075, 81080, |
| | 80980, 80985, | 81123-81148 | 81083, 81149- |
| | 80988, 80991, | | 81180 |
| | 81087-81122 | | |
| 12:40350028_G_A | 81181-81228 | 81229-81277 | 81278-81329 |
| rs4768234_REF | | | |
| 12:40350028_G_A | 81182, 81184, | 81230, 81232, | 81279, 81281, |
| rs4768234_SNP | 81188, 81194, | 81236, 81246- | 81287, 81291, |
| | 81198-81199, | 81247, 81256, | 81297-81298, |
| | 81212, 81216, | 81261, 81271, | 81305, 81308, |
| | 81222, 81227, | 81276, 81366- | 81323, 81328, |
| | 81330-81365 | 81395 | 81396-81435 |
| 12:40350284_A_T | 81436-81447 | 81448-81459 | 81460-81474 |
| rs4768235_REF | | | |
| 12:40350284_A_T | 81436-81437, | 81448-81450, | 81460-81462, |
| rs4768235_SNP | 81439, 81441- | 81452-81453, | 81464, 81466- |
| | 81444, 81446- | 81455, 81457- | 81467, 81469, |
| | 81447, 81475- | 81459, 81478- | 81471, 81473- |
| | 81477 | 81480 | 81474, 81481- |
| | | | 81485 |
| 12:40350343_C_A | 81486-81533 | 81534-81583 | 81584-81635 |
| rs7314455_REF | | | |
| 12:40350343_C_A | 81489, 81492, | 81537, 81540- | 81590-81591, |
| rs7314455_SNP | 81495, 81500- | 81541, 81544, | 81594, 81602, |
| | 81501, 81509- | 81552, 81559- | 81609-81611, |
| | 81510, 81513, | 81560, 81563, | 81616, 81618, |
| | 81515, 81517, | 81565, 81567, | 81628, 81714- |
| | 81636-81673 | 81674-81713 | 81755 |
| 12:40350381_G_A | 81756-81803 | 81804-81853 | 81854-81905 |
| rs7313525_REF | | | |
| 12:40350381_G_A | 81758-81759, | 81806, 81815, | 81856, 81874, |
| rs7313525_SNP | 81767, 81776, | 81824, 81828, | 81878, 81883, |
| | 81779, 81786, | 81833, 81836, | 81886, 81894, |
| | 81794-81795, | 81844-81845, | 81896, 81898, |
| | 81801, 81803, | 81851, 81853, | 81903, 81905, |
| | 81906-81943 | 81944-81983 | 81984-82025 |
| 12:40350592_G_T | 82026-82046 | 82047-82066 | 82067-82088 |
| rs7313895_REF | | | |
| 12:40350592_G_T | 82031-82032, | 82053, 82059- | 82071, 82074, |
| rs7313895_SNP | 82040, 82044, | 82060, 82064, | 82082, 82086, |
| | 82046, 82089- | 82066, 82102- | 82088, 82113- |
| | 82101 | 82112 | 82125 |
| 12:40350689_C_T | 82126-82137 | 82138-82142 | 82143-82149 |
| rs7314863_REF | | | |
| 12:40350689_C_T | 82126-82128, | | |
| rs7314863_SNP | 82150-82152 | | |
| 12:40350973_A_C | 82153-82200 | 82201-82250 | 82251-82302 |
| rs7303525_REF | | | |
| 12:40350973_A_C | 82158, 82164, | 82212, 82216, | 82261, 82263, |
| rs7303525_SNP | 82169, 82184, | 82231, 82234, | 82267, 82277, |
| | 82186, 82191, | 82236, 82241, | 82283, 82288, |
| | 82194, 82198- | 82244, 82248- | 82293, 82300- |
| | 82200, 82303- | 82250, 82341- | 82302, 82381- |
| | 82340 | 82380 | 82422 |
| 12:40351006_G_A | 82423-82469 | 82470-82519 | 82520-82571 |
| rs7132187_REF | | | |
| 12:40351006_G__A | 82425, 82439- | 82472-82473, | 82522-82523, |
| rs7132187_SNP | 82441, 82443, | 82489, 82491, | 82539, 82541, |
| | 82448, 82458, | 82496-82497, | 82547, 82550, |
| | 82467, 82469, | 82505, 82508, | 82556, 82568- |
| | 82572-82609 | 82517, 82519, | 82569, 82571, |
| | | 82610-82649 | 82650-82691 |
| 12:40351379_T_C | 82692 | | 82693 |
| rs7307310_REF | | | |
| 12:40351379_T_C | 82692 | | 82693 |
| rs7307310_SNP | | | |
| 12:40352036_T_C | 82694-82740 | 82741-82790 | 82791-82842 |
| rs11176153_REF | | | |
| 12:40352036_T_C | 82694-82695, | 82741-82742, | 82791, 82800, |
| rs11176153_SNP | 82697, 82703, | 82750, 82753, | 82803, 82806, |
| | 82716, 82719, | 82756, 82765, | 82815, 82818, |
| | 82723-82724, | 82768, 82772, | 82822-82824, |
| | 82728, 82737, | 82777, 82786, | 82829, 82921- |
| | 82843-82880 | 82881-82920 | 82962 |
| 12:40352100_G_C | 82963-83009 | 83010-83059 | 83060-83111 |
| rs11176154_REF | | | |
| 12:40352100_G_C | 82980, 82982- | 83027, 83029- | 83068, 83078, |
| rs11176154_SNP | 82984, 82994, | 83031, 83033, | 83080-83083, |
| | 83001, 83004- | 83041, 83050- | 83085, 83093, |
| | 83005, 83009, | 83051, 83055, | 83102-83103, |
| | 83112-83149 | 83059, 83150- | 83190-83231 |
| | | 83189 | |
| 12:40352112_T_C | 82965, 82968- | 83012, 83015- | 83062, 83065- |
| rs7311247_REF | 82971, 82973, | 83018, 83020, | 83067, 83069, |
| | 82977-82982, | 83024-83029, | 83071, 83075- |
| | 82985, 82989- | 83032-83033, | 83081, 83084- |
| | 82992, 82995- | 83035, 83037- | 83085, 83087, |
| | 82996, 82999, | 83040, 83044- | 83089-83092, |
| | 83004, 83006, | 83045, 83048, | 83096-83097, |
| | 83009, 83232- | 83050, 83054, | 83100, 83102, |
| | 83255 | 83056, 83059, | 83105-83106, |
| | | 83256-83279 | 83108, 83111, |
| | | | 83280-83303 |
| 12:40352112_T_C | 82965, 82971, | 83012, 83018, | 83062, 83071, |
| rs7311247_SNP | 82973, 82995, | 83020, 83035, | 83087, 83100, |
| | 82999, 83232- | 83048, 83256- | 83105, 83280- |
| | 83233, 83237, | 83257, 83261, | 83281, 83286, |
| | 83245, 83247, | 83269, 83275, | 83293, 83299, |
| | 83304-83341 | 83342-83381 | 83382-83423 |
| 12:40352153_C_T | 83424-83470 | 83471-83514 | 83515-83563 |
| rs7137437_REF | | | |
| 12:40352153_C_T | 83428, 83430, | 83475, 83485, | 83521, 83530, |
| rs7137437_SNP | 83439, 83444- | 83487, 83491- | 83532, 83536- |
| | 83445, 83448, | 83492, 83500, | 83538, 83540, |
| | 83451, 83454, | 83506, 83595- | 83548, 83554, |
| | 83461, 83564- | 83617 | 83618-83646 |
| | 83594 | | |
| 12:40352235_AG_A | 83647-83694 | 83695-83742 | 83743-83793 |
| rs75439407_REF | | | |
| 12:40352235_AG_A | 83647, 83652, | 83695, 83700, | 83743, 83748, |
| rs75439407_SNP | 83657, 83669, | 83705, 83721, | 83753, 83770- |
| | 83675-83676, | 83724, 83728, | 83771, 83775, |
| | 83680, 83690, | | |
| | 83692, 83794- | 83738, 83828- | 83778, 83791, |
| | 83827 | 83861 | 83862-83899 |
| 12:40352271_T_C | 83900-83947 | 83948-83997 | 83998-84049 |
| rs73107108_REF | | | |
| 12:40352271_T_C | 83900, 83902, | 83948, 83956, | 84006, 84008, |
| rs73107108_SNP | 83908, 83910, | 83958, 83963, | 84013, 84015- |
| | 83915, 83919, | 83965, 83968, | 84016, 84028, |
| | 83928, 83930, | 83977, 83979, | 84030, 84033, |
| | 83935-83936, | 83984, 83994, | 84036, 84046, |
| | 84050-84087 | 84088-84127 | 84128-84169 |
| 12:40352469_C_CTTTTTTTTT | 84170-84183 | 84184-84190 | 84191-84201 |
| TTTTTT | | | |
| rs1307806388_REF | | | |
| 12:40352469_C_CTTTTTTTTT | 84170-84183 | 84184-84190 | 84191-84201 |
| TTTTT | | | |
| rs1307806388_REF | | | |
| 12:40352469_C_CTTTTTTTTT | 84170-84183 | 84184-84190 | 84191-84201 |
| TTTT | | | |
| rs1307806388_REF | | | |
| 12:40352469_C_CT | 84170-84183 | 84184-84190 | 84191-84201 |
| rs1307806388_REF | | | |
| 12:40352707_T_G | 84202-84244 | 84245-84294 | 84295-84346 |
| rs10878409_SNP | | | |
| 12:40352874_C_T | 84347-84357 | 84358-84368 | 84369-84381 |
| rs10878410_SNP | | | |
| 12:40352927_G_C | 84382-84403 | 84404-84427 | 84428-84453 |
| rs138382676_REF | | | |
| 12:40352927_G_C | 84385-84386, | 84407-84409, | 84431-84433, |
| rs138382676_SNP | 84393, 84399, | 84423, 84483- | 84443, 84517- |
| | 84454-84482 | 84516 | 84554 |
| 12:40353080_G_A | A | , | 84555 |
| rs11176161_SNP | | | |
| 12:40353111_T_C | 84556-84588 | 84589-84623 | 84624-84660 |
| rs10878412_REF | | | |
| 12:40353111_T_C | 84557-84558, | 84591, 84605- | 84626, 84642, |
| rs10878412_SNP | 84571-84572, | 84606, 84610, | 84646, 84653, |
| | 84576, 84661- | 84621, 84683- | 84658, 84688- |
| | 84682 | 84687 | 84701 |
| 12:40353158_T_C | , | 84702 | 84703-84705 |
| rs10878413_REF | | | |
| 12:40353186_C_T | 84706-84716 | 84717-84725 | 84726-84736 |
| rs183544696_REF | | | |
| 12:40353186_C_T | 84737-84776 | 84777-84813 | 84814-84853 |
| rs183544696_SNP | | | |
| 12:40353191_C_T | 84706-84716 | 84717-84725 | 84726-84736 |
| rs189538849_REF | | | |
| 12:40353191_C_T | 84854-84886 | 84887-84913 | 84914-84943 |
| rs189538849_SNP | | | |
| 12:40353217_C_T | 84944-84951 | 84952-84961 | 84962-84975 |
| rs11610569_SNP | | | |
| 12:40353226_C_T | 84976-84988 | 84989-85003 | 85004-85020 |
| rs142726158_REF | | | |
| 12:40353226_C_T | 84979-84981, | 84991, 84993- | 85006, 85008- |
| rs142726158_SNP | 84983, 84985, | 84995, 84999, | 85009, 85014- |
| | 85021-85038 | 85039-85058 | 85015, 85059- |
| | | | 85080 |
| 12:40353242_CAAA_C | 84976-84988, | 84989-85003, | 85004-85020, |
| rs1194726090_REF | 85081-85105 | 85106-85132 | 85133-85159 |
| 12:40353242_CAAA_C | 84977-84978, | 84992, 84996, | 85011, 85016- |
| rs1194726090_SNP | 84986, 85160- | 85000, 85189- | 85017, 85213- |
| | 85188 | 85212 | 85239 |
| 12:40353243_A_G | 84976-84988, | 84989-85003, | 85004-85020, |
| rs576444406_REF | 85081-85105 | 85106-85132 | 85133-85159, |
| | | | 85240 |
| 12:40353243_A_G | 84977-84978, | 84992, 84996, | 85011, 85016- |
| rs576444406_SNP | 84982, 84986, | 85000-85001, | 85018, 85156, |
| | 85103, 85174, | 85116, 85191, | 85220, 85311- |
| | 85241-85274 | 85275-85310 | 85348 |
| 12:40353244_A_G | 84976-84986, | 84989-85001, | 85004-85018, |
| rs185723137_REF | 84988, 85081- | 85003, 85106- | 85020, 85133- |
| | 85105 | 85132 | 85159, 85240, |
| | | | 85349 |
| 12:40353246_GGGGCTCCTCA | 84976-84981, | 84989-84995, | 85004-85011, |
| CTTCTCAGACGGGGCGGCT | 84984-84985, | 84998-85000, | 85013-85016, |
| GCCGGGCA_G | 85081-85105 | 85106-85132 | 85133-85159, |
| rs1179215763_REF | | | 85240, 85349 |
| 12:40353246_GGGGCTCCTCA | 84976, 84984, | 84989-84990, | 85004-85005, |
| CTTCTCAGACGGGGCGGCT | 85091, 85100- | 84998, 85115, | 85007, 85013, |
| GCCGGGCA_G | 85101, 85350- | 85128, 85390- | 85142, 85432- |
| rs1179215763_SNP | 85389 | 85431 | 85475 |
| 12:40353266_C_T | , | , | 85240, 85349 |
| rs71449759_REF | | | |
| 12:40353266_C_T | 85476-85478 | 85479-85483 | 85349, 85484- |
| rs71449759_SNP | | | 85489 |
| 12:40353289_A_G | 85490-85494 | 85495-85501 | 85502-85510 |
| rs61915613_REF | | | |
| 12:40353344_A_G | 85511-85525 | 85526-85542 | 85543-85561 |
| rs140722234_REF | | | |
| 12:40353344_A_G | 85513-85514, | 85528-85529, | 85545-85546, |
| rs140722234_SNP | 85520, 85523- | 85538-85540, | 85556-85557, |
| | 85524, 85562- | 85572-85580 | 85559, 85581- |
| | 85571 | | 85592 |
| 12:40353383_C_T | 85593-85597 | 85598-85604 | 85605-85613 |
| rs191099092_REF | | | |
| 12:40353383_C_T | 85593-85594, | 85598-85599, | 85605-85606, |
| rs191099092_SNP | 85597, 85614- | 85603-85604, | 85611-85613, |
| | 85621 | 85622-85632 | 85633-85646 |
| 12:40353440_C_A | 85647-85686 | 85687-85726 | 85727-85770 |
| rs113693842_REF | | | |
| 12:40353440_C_A | 85655, 85683, | 85695, 85700, | 85735, 85740- |
| rs113693842_SNP | 85771-85775 | 85724, 85776- | 85741, 85768, |
| | | 85786 | 85787-85803 |
| 12:40353467_C_T | 85804-85809 | 85810-85818 | 85819-85829 |
| rs112767992_REF | | | |
| 12:40353467_C_T | 85804-85805, | 85810-85811, | 85820, 85823, |
| rs112767992_SNP | 85807, 85809, | 85814, 85816, | 85825, 85827, |
| | 85830-85835 | 85818, 85836- | 85829, 85845- |
| | | 85844 | 85857 |
| 12:40353515_AG_A | 85858-85884 | 85885-85908 | 85909-85939 |
| rs34531686_REF | | | |
| 12:40353632_C_T | 85940 | 85941-85943 | 85944-85948 |
| rs12368227_REF | | | |
| 12:40353632_C_T | 85940 | 85941, 85943, | 85944-85945, |
| rs12368227_SNP | | 85949 | 85948, 85950- |
| | | | 85951 |
| 12:40353660_C_A | 85952-85986 | 85987-86022 | 86023-86061 |
| rs12368229_REF | | | |
| 12:40353660_C_A | 85953, 85960, | 85994, 85996, | 86031, 86038- |
| rs12368229_SNP | 85965, 85968, | 86001, 86004, | 86039, 86042, |
| | 85983, 86062- | 86019, 86097- | 86058, 86134- |
| | 86096 | 86133 | 86173 |
| 12:40353732_G_C | , | 86174 | 86175-86176 |
| rs3930031_SNP | | | |
| 12:40354127_C_T | 86177-86221 | 86222-86264 | 86265-86312 |
| rs7308626_REF | | | |
| 12:40354127_C_T | 86189, 86193, | 86233, 86238, | 86273, 86280, |
| rs7308626_SNP | 86195, 86201, | 86244, 86248, | 86285-86286, |
| | 86205, 86211, | 86254, 86259, | 86292, 86296, |
| | 86213, 86313- | 86346-86370 | 86302, 86307, |
| | 86345 | | 86371-86401 |
| 12:40354593_G_A | 86402-86423 | 86424-86430 | 86431-86444 |
| rs890575_REF | | | |
| 12:40354593_G_A | 86409-86410, | 86425, 86427, | 86435, 86438, |
| rs890575_SNP | 86412, 86419- | 86448-86450 | 86442, 86451- |
| | 86420, 86445- | | 86455 |
| | 86447 | | |
| 12:40354745_A_G | 86456-86503 | 86504-86553 | 86554-86605 |
| rs10748040_REF | | | |
| 12:40354745_A_G | 86459, 86462, | 86507, 86510, | 86557, 86560, |
| rs10748040_SNP | 86467-86468, | 86515-86516, | 86565, 86572, |
| | 86476, 86478, | 86522, 86525, | 86575-86576, |
| | 86482, 86484, | 86527, 86533- | 86584-86585, |
| | 86489, 86492, | 86534, 86539, | 86590, 86592, |
| | 86606-86643 | 86644-86683 | 86684-86725 |
| 12:40354835_AAAAAAAAAA | 86726-86727 | | |
| TATAT_A | | | |
| rs61230125_REF | | | |
| 12:40354835_AAAAAAAAAA | 86726-86727 | | |
| TATAT_A | | | |
| rs61230125_SNP | | | |
| 12:40354862_A_C | 86728-86743 | 86744-86757 | 86758-86773 |
| rs11176165_REF | | | |
| 12:40354862_A_C | 86728, 86730, | 86744-86745, | 86758-86759, |
| rs11176165_SNP | 86735-86736, | 86749, 86752, | 86766, 86768, |
| | 86738, 86774- | 86754, 86789- | 86773, 86802- |
| | 86788 | 86801 | 86816 |
| 12:40355348_G_A | 86817-86864 | 86865-86914 | 86915-86966 |
| rs7976715_REF | | | |
| 12:40355348_G_A | 86817-86818, | 86865-86867, | 86915-86916, |
| rs7976715_SNP | 86820-86821, | 86870, 86872, | 86918, 86921, |
| | 86823, 86830, | 86879, 86887, | 86923, 86928, |
| | 86838, 86850, | 86898, 86900, | 86931, 86939, |
| | 86853, 86863, | 86903, 87005- | 86950, 86955, |
| | 86967-87004 | 87044 | 87045-87086 |
| 12:40355370_G_A | 86823, 86837, | 86872, 86874, | 86919, 86923, |
| rs7976724_REF | 86843, 86853, | 86886, 86892, | 86925, 86928, |
| | 87087-87130 | 86898, 86903, | 86938, 86944, |
| | | 87131-87174 | 86950, 86955, |
| | | | 87175-87218 |
| 12:40355370_G_A | 86837, 86843, | 86874, 86886, | 86919, 86925, |
| rs7976724_SNP | 87095, 87101, | 86892, 87145, | 86938, 86944, |
| | 87105, 87109, | 87149, 87153, | 87192, 87196- |
| | 87112, 87124, | 87156, 87169, | 87197, 87200, |
| | 87126, 87130, | 87173-87174, | 87213, 87218, |
| | 87219-87256 | 87257-87296 | 87297-87338 |
| 12:40355395_A_G | 87339-87385 | 87386-87435 | 87193, 87197, |
| rs7963987_REF | | | 87436-87485 |
| 12:40355395_A_G | 87342, 87344, | 87389, 87394, | 87193, 87439, |
| rs7963987_SNP | 87347, 87351, | 87398, 87401, | 87444, 87448, |
| | 87354, 87362, | 87410, 87422, | 87458, 87461, |
| | 87373, 87375- | 87424-87426, | 87472, 87474- |
| | 87376, 87381, | 87431, 87524- | 87476, 87564- |
| | 87486-87523 | 87563 | 87605 |
| 12:40355416_G_C | 87341-87342, | 87388-87389, | 87437-87439, |
| rs2162472_REF | 87351, 87366, | 87397-87398, | 87447-87448, |
| | 87372, 87376, | 87414, 87420, | 87458, 87464, |
| | 87606-87629 | 87424, 87426, | 87470, 87474, |
| | | 87630-87654 | 87476, 87655- |
| | | | 87679 |
| 12:40355416_G_C | 87341, 87366, | 87388, 87397, | 87437-87438, |
| rs2162472_SNP | 87372, 87609, | 87414, 87420, | 87447, 87464, |
| | 87623, 87680- | 87634, 87714- | 87470, 87752- |
| | 87713 | 87751 | 87792 |
| 12:40355505_A_ATCCCTCCC | 87793-87807 | 87808-87824 | 87825-87843 |
| TCCCTCCC | | | |
| rs767501990_REF | | | |
| 12:40355505_A_ATCCCTCCC | 87793-87807 | 87808-87824 | 87825-87843 |
| TCCC | | | |
| rs767501990_REF | | | |
| 12:40355505_A_ATCCCTCCC | 87793-87807 | 87808-87824 | 87825-87843 |
| rs767501990_REF | | | |
| 12:40355505_A_ATCCC | 87793-87807 | 87808-87824 | 87825-87843 |
| rs767501990_REF | | | |
| 12:40355543_T_TCCTTC | 87844-87845 | 87846-87849 | 87850-87855 |
| rs552326490_REF | | | |
| 12:40355543_T_TC | 87844-87845 | 87846-87849 | 87850-87855 |
| rs552326490_REF | | | |
| 12:40355544_CTT_C | 87844-87845 | 87846-87849 | 87850-87855 |
| rs1365381164_REF | | | |
| 12:40355546_T_TC | 87844-87845 | 87846-87849 | 87850-87855 |
| rs760911090_REF | | | |
| 12:40355546_T_TC | 87856 | 87857 | 87858-87860 |
| rs760911090_SNP | | | |
| 12:40355549_T_TC | 87844-87845 | 87846-87849 | 87850-87855 |
| rs754028820_REF | | | |
| 12:40356669_G_A | 87861-87908 | 87909-87958 | 87959-88010 |
| rs11564173_REF | | | |
| 12:40356669_G_A | 87872, 87874, | 87918, 87921, | 87961, 87969, |
| rs11564173_SNP | 87883, 87886, | 87927, 87933, | 87972, 87978- |
| | 87891, 87896, | 87936, 87941, | 87979, 87985, |
| | 87900, 87906- | 87946, 87950, | 87988, 87993, |
| | 87908, 88011- | 87957-87958, | 88002, 88010, |
| | 88048 | 88049-88088 | 88089-88130 |
| 12:40356747_T_A | 88131-88142 | 88143-88148 | 88149-88158 |
| rs7306545_REF | | | |
| 12:40356747_T_A | 88135-88136, | 88147-88148, | 88152-88153, |
| rs7306545_SNP | 88140-88142, | 88166-88169 | 88156-88157, |
| | 88159-88165 | | 88170-88175 |
| 12:40357018_A_G | 88176 | | 88177 |
| rs7306684_REF | | | |
| 12:40357018_A_G | 88178-88216 | 88217-88248 | 88249-88284 |
| rs7306684_SNP | | | |
| 12:40357164_C_T | | | 88285 |
| rs112259589_REF | | | |
| 12:40357164_C_T | 88286-88300 | 88301-88319 | 88285, 88320- |
| rs112259589_SNP | | | 88341 |
| 12:40357182_A_G | 88342-88358 | 88359-88375 | 88285, 88376- |
| rs73277531_REF | | | 88399 |
| 12:40357182_A_G | 88400-88436 | 88437-88475 | 88383, 88476- |
| rs73277531_SNP | | | 88517 |
| 12:40357363_CTT_C | 88518-88534 | 88535-88550 | 88551-88568 |
| rs77689380_REF | | | |
| 12:40357363_CTT_C | 88526, 88531, | 88542-88543 | 88560-88561, |
| rs77689380_SNP | 88569 | | 88566, 88570 |
| 12:40357725_G_A | 88571-88606 | 88607-88643 | 88644-88683 |
| rs73277533_REF | | | |
| 12:40357725_G_A | 88571-88572, | 88607, 88621, | 88659, 88666, |
| rs73277533_SNP | 88590, 88600, | 88628, 88718- | 88675, 88737- |
| | 88684-88717 | 88736 | 88759 |
| 12:40357968_A_G | 88760-88798 | 88799-88839 | 88840-88882 |
| rs17462055_REF | | | |
| 12:40357968_A_G | 88794-88798, | 88835-88839, | 88850, 88879- |
| rs17462055_SNP | 88883-88919 | 88920-88960 | 88882, 88961- |
| | | | 89005 |
| 12:40358092_C_CT | 89006-89013 | 89014-89021 | 89022-89031 |
| rs758880647_REF | | | |
| 12:40358177_T_C | 89032-89073 | 89074-89102 | 89103-89137 |
| rs10784528_REF | | | |
| 12:40358177_T_C | 89042, 89047, | 89078, 89083, | 89104, 89108, |
| rs10784528_SNP | 89053, 89058, | 89090, 89098, | 89113, 89121, |
| | 89063, 89068, | 89175-89211 | 89127, 89131, |
| | 89071, 89138- | | 89212-89250 |
| | 89174 | | |
| 12:40358735_AT_A | 89251-89260 | 89261-89266 | 89267-89274 |
| rs796949072_REF | | | |
| 12:40358801_A_T | | 89275 | 89276-89277 |
| rs10784532_REF | | | |
| 12:40358801_A_T | 89278-89285 | 89275, 89286- | 89276-89277, |
| rs_10784532_SNP | | 89292 | 89293-89303 |
| 12:40359018_TTTG_T | 89304-89333 | 89334-89349 | 89350-89371 |
| rs145580704_REF | | | |
| 12:40359018_TTTG_T | 89317, 89322, | 89344, 89346, | 89367, 89369- |
| rs145580704_SNP | 89326, 89330, | 89348-89349, | 89371, 89376- |
| | 89372-89374 | 89375 | 89377 |
| 12:40359242_C_CT | 89378-89396 | 89397-89415 | 89416-89434 |
| rs386376254_REF | | | |
| 12:40359242_C_CT | 89389 | 89410 | 89432 |
| rs386376254_SNP | | | |
| 12:40359972_C_T | 89435-89446 | 89447-89457 | 89458-89470 |
| rs11836288_REF | | | |
| 12:40359972_C_T | 89435, 89471- | | |
| rs11836288_SNP | 89479 | | |
| 12:40360506_T_C | 89480-89527 | 89528-89577 | 89578-89629 |
| rs11564146_REF | | | |
| 12:40360506_T_C | 89482, 89488, | 89530, 89536, | 89580, 89586, |
| rs11564146_SNP | 89495, 89500, | 89543, 89548, | 89594, 89599, |
| | 89502, 89504, | 89550, 89552, | 89601, 89609, |
| | 89510, 89519, | 89558, 89566, | 89613, 89618, |
| | 89522, 89527, | 89568, 89570, | 89620, 89622, |
| | 89630-89667 | 89668-89707 | 89708-89749 |
| 12:40360610_A_G | 89750-89784 | 89785-89812 | 89813-89846 |
| rs10784536_REF | | | |
| 12:40360610_A_G | 89757-89760, | 89786, 89789- | 89816, 89819, |
| rs10784536_SNP | 89763, 89765, | 89793, 89797- | 89821-89822, |
| | 89772, 89774, | 89798, 89802, | 89828-89829, |
| | 89776, 89778, | 89804, 89883- | 89832, 89834, |
| | 89847-89882 | 89914 | 89836, 89842, |
| | | | 89915-89954 |
| 12:40360877_CTGCCTAT_C | 89955-90001 | 90002-90051 | 90052-90103 |
| rs11277474_REF | | | |
| 12:40360877_CTGCCTAT_C | 89957, 89971, | 90004, 90019, | 90055, 90070, |
| rs11277474_SNP | 89974, 89984, | 90022, 90038, | 90074, 90090, |
| | 89997, 90104- | 90047, 90144- | 90101, 90186- |
| | 90143 | 90185 | 90229 |
| 12:40360895_G_A | 89967-89970, | 90009, 90012, | 90053, 90060, |
| rs7971919_REF | 89981-89982, | 90015-90018, | 90063, 90066- |
| | 89986-89987, | 90030-90031, | 90069, 90082- |
| | 89994-89995, | 90035-90037, | 90083, 90087- |
| | 89998, 90108- | 90044-90045, | 90089, 90092, |
| | 90109, 90114, | 90048, 90148- | 90096-90097, |
| | 90125, 90127, | 90149, 90155, | 90100, 90190- |
| | 90230-90260 | 90166, 90168, | 90191, 90197, |
| | | 90261-90291 | 90208, 90220, |
| | | | 90292-90322 |
| 12:40360895_G_A | 89967-89969, | 90012, 90015- | 90063, 90066, |
| rs7971919_SNP | 89981, 89994, | 90016, 90030, | 90082, 90092, |
| | 90232, 90245, | 90044, 90261, | 90096, 90292, |
| | 90248, 90250, | 90264, 90276, | 90294, 90306, |
| | 90257, 90323- | 90279, 90288, | 90309, 90315, |
| | 90360 | 90361-90400 | 90401-90442 |
| 12:40360980_T_G | 90443-90449 | 90450-90453 | 90454-90460 |
| rs7962116_REF | | | |
| 12:40360980_T_G | 90443-90449, | 90450-90453, | 90454-90460, |
| rs7962116_SNP | 90461-90474 | 90475-90477 | 90478-90486 |
| 12:40361242_TTACA_T | 90487-90488 | | |
| rs67512688_REF | | | |
| 12:40361242_TTACA_T | 90487-90488 | | |
| rs67512688_SNP | | | |
| 12:40361614_G_A | 90489-90517 | 90518-90530 | 90531-90550 |
| rs2896978_REF | | | |
| 12:40361614_G_A | 90498, 90516- | | 90535, 90558- |
| rs2896978_SNP | 90517, 90551- | | 90559 |
| | 90557 | | |
| 12:40361674_A_T | 90560-90607 | 90608-90657 | 90658-90709 |
| rs2404836_REF | | | |
| 12:40361674_A_T | 90564, 90569- | 90612, 90614, | 90662, 90664, |
| rs2404836_SNP | 90570, 90574, | 90616, 90619- | 90666, 90669, |
| | 90580, 90586, | 90620, 90624, | 90673, 90675, |
| | 90588, 90593, | 90636, 90638, | 90685, 90688, |
| | 90597, 90600, | 90643, 90647, | 90690, 90695, |
| | 90710-90747 | 90748-90787 | 90788-90829 |
| 12:40362405_T_C | 90830-90876 | 90877-90926 | 90927-90978 |
| rs11176195_REF | | | |
| 12:40362405_T_C | 90840, 90842, | 90888, 90890, | 90931, 90937, |
| rs11176195_SNP | 90856, 90858- | 90894, 90907- | 90940, 90942, |
| | 90860, 90866, | 90910, 90916, | 90946, 90960- |
| | 90869, 90871, | 90921, 90925, | 90962, 90968, |
| | 90875, 90979- | 91017-91056 | 90973, 91057- |
| | 91016 | | 91098 |
| 12:40362446_A_G | 91099-91146 | 91147-91192 | 91193-91242 |
| rs4767973_REF | | | |
| 12:40362446_A_G | 91103, 91108, | 91151, 91156, | 91197, 91207- |
| rs4767973_SNP | 91114, 91118, | 91161-91162, | 91208, 91212, |
| | 91120-91122, | 91166, 91169- | 91216, 91227, |
| | 91130, 91139, | 91170, 91181, | 91234-91235, |
| | 91145, 91243- | 91186, 91191, | 91239, 91241, |
| | 91280 | 91281-91320 | 91321-91362 |
| 12:40362519_C_T | 91363-91404 | 91405-91438 | 91439-91477 |
| rs17444202_REF | | | |
| 12:40362519_C_T | 91369, 91384, | 91411, 91425, | 91450, 91461, |
| rs17444202_SNP | 91387, 91393, | 91434, 91501- | 91467, 91473, |
| | 91400, 91478- | 91507 | 91508-91522 |
| | 91500 | | |
| 12:40362670_C_A | 91523-91570 | 91571-91619 | 91620-91671 |
| rs4768236_REF | | | |
| 12:40362670_C_A | 91535, 91540, | 91583, 91588, | 91633, 91638, |
| rs4768236_SNP | 91543, 91546, | 91591, 91594, | 91641, 91644, |
| | 91558-91559, | 91603, 91608- | 91653, 91657, |
| | 91562, 91566, | 91609, 91612, | 91659-91660, |
| | 91569-91570, | 91619, 91710- | 91663, 91671, |
| | 91672-91709 | 91749 | 91750-91791 |
| 12:40362885_T_C | 91792-91814 | 91815-91825 | 91826-91843 |
| rs4768237_REF | | | |
| 12:40362885_T_C | 91795-91796, | 91815-91816, | 91827-91828, |
| rs4768237_SNP | 91799, 91801, | 91818-91820, | 91831, 91835, |
| | 91803-91804, | 91822-91825, | 91837-91839, |
| | 91808-91809, | 91866-91883 | 91841-91843, |
| | 91811, 91813, | | 91884-91905 |
| | 91844-91865 | | |
| 12:40363312_A_ACATG | 91906-91907 | | |
| rs58724345_SNP | | | |
| 12:40363384_AT_A | 91908-91913 | | |
| rs397897325_REF | | | |
| 12:40363384_AT_A | 91910, 91913- | | |
| rs397897325_SNP | 91914 | | |
| 12:40363528_A_G | 26687, 26689, | 26729, 26731, | 26770, 26772, |
| rs33962975_REF | 26694, 26704, | 26733, 26738, | 26774, 26776, |
| | 26720, 91915- | 26747, 91954- | 26781, 91996- |
| | 91953 | 91995 | 92039 |
| 12:40363528_A_G | 91916, 91918- | 91955, 91957, | 91996, 91998, |
| rs33962975_SNP | 91919, 91921, | 91959, 91964, | 92002, 92006, |
| | 91923, 91926, | 91976, 91985, | 92018, 92029, |
| | 91938, 91946, | 91994, 92078- | 92038, 92118- |
| | 92040-92077 | 92117 | 92159 |
| 12:40363731_A_G | 92160-92179 | 92180-92185 | 92186-92195 |
| rs7134408_REF | | | |
| 12:40363731_A_G | 92160, 92163, | 92185, 92234- | 92186, 92259- |
| rs7134408_SNP | 92170, 92172, | 92258 | 92292 |
| | 92177, 92196- | | |
| | 92233 | | |
| 12:40364243_T_G | 92293-92339 | 92340-92381 | 92382-92429 |
| rs11835105_REF | | | |
| 12:40364243_T_G | 92296, 92298- | 92341, 92343- | 92384, 92388, |
| rs11835105_SNP | 92300, 92304, | 92345, 92353- | 92396-92398, |
| | 92306, 92309- | 92355, 92370- | 92404, 92408, |
| | 92311, 92328, | 92371, 92379, | 92417-92418, |
| | 92430-92467 | 92468-92507 | 92426, 92508- |
| | | | 92549 |
| 12:40364289_T_TA | 92550-92597 | 92598-92647 | 92648-92699 |
| rs137858615_REF | | | |
| 12:40364289_T_TA | 92550-92551, | 92598, 92602, | 92648, 92652, |
| rs137858615_SNP | 92554, 92556, | 92604, 92608, | 92654, 92659, |
| | 92571, 92573- | 92619, 92621, | 92672, 92680, |
| | 92574, 92577, | 92625, 92629, | 92685, 92689, |
| | 92589, 92700- | 92638, 92738- | 92691, 92778- |
| | 92737 | 92777 | 92819 |
| 12:40364489_G_A | 92820-92859 | 92860-92880 | 92881-92911 |
| rs10878434_REF | | | |
| 12:40364489_G_A | 92825, 92829, | 92927-92930 | 92895, 92931- |
| rs10878434_SNP | 92838, 92840, | | 92938 |
| | 92846, 92912- | | |
| | 92926 | | |
| 12:40364516_C_CT | 92939-92942 | 92943 | 92944-92945 |
| rs35403247_REF | | | |
| 12:40364850_T_C | 26835, 26846, | 26978, 26980, | 26904, 26907, |
| rs3761863_REF | 26939, 26949, | 26998, 92980- | 27019, 27037, |
| | 26959, 92946- | 93009 | 27039, 93010- |
| | 92979 | | 93044 |
| 12:40364850_T_C | 26939, 26949, | 26978, 26980, | 27019, 27037, |
| rs3761863_SNP | 26959, 92952, | 26998, 92986, | 27039, 93011, |
| | 92958, 92972, | 92989, 93006, | 93017, 93033, |
| | 92976, 93045- | | |
| | 93082 | 93083-93122 | 93123-93164 |
| 12:40365793_G_A | 93165-93210 | 93211-93253 | 93254-93300 |
| rs17444223_REF | | | |
| 12:40365793_G_A | 93180, 93185, | 93222, 93237- | 93260, 93266, |
| rs17444223_SNP | 93187, 93192- | 93238, 93241, | 93283, 93286, |
| | 93193, 93196, | 93249, 93330- | 93296, 93343- |
| | 93201, 93206, | 93342 | 93367 |
| | 93209, 93301- | | |
| | 93329 | | |
| 12:40366046_T_A | 93368-93409 | 93410-93443 | 93444-93483 |
| rs12426362_REF | | | |
| 12:40366046_T_A | 93371, 93377, | 93412, 93415, | 93447, 93453, |
| rs12426362_SNP | 93391, 93399, | 93427, 93521- | 93474, 93552- |
| | 93406, 93484- | 93551 | 93588 |
| | 93520 | | |
| 12:40366178_A_G | 93589-93635 | 93636-93685 | 93686-93737 |
| rs17466605_REF | | | |
| 12:40366178_A_G | 93591-93592, | 93638, 93648, | 93688, 93695, |
| rs17466605_SNP | 93601, 93605- | 93652-93653, | 93699-93700, |
| | 93606, 93621- | 93666, 93669- | 93704-93705, |
| | 93622, 93625, | 93670, 93673, | 93718, 93721, |
| | 93633, 93738- | 93682-93683, | 93725, 93734, |
| | 93775 | 93776-93815 | 93816-93857 |
| 12:40367205_A_G | 93858-93867 | , | 93868-93869 |
| rs3789328_REF | | | |
| 12:40367205_A_G | 93858-93859, | 93897-93907 | 93908-93924 |
| rs3789328_SNP | 93863-93864, | | |
| | 93867, 93870- | | |
| | 93896 | | |
| 12:40367513_A_T | 93925-93934 | 93935-93938 | 93939-93944 |
| rs12426498_REF | | | |
| 12:40367513_A_T | 93925, 93945- | 93954-93957 | 93958-93963 |
| rs12426498_SNP | 93953 | | |
| 12:40367556_A_G | 93964-93977 | 93978-93987 | 93988-94001 |
| rs4768238_REF | | | |
| 12:40367556_A_G | 93965, 93967, | 93979, 93983, | 93989, 93991, |
| rs4768238_SNP | 93972, 93974, | 93985, 93987, | 93997, 93999, |
| | 93977, 94002- | 94027-94037 | 94001, 94038- |
| | 94026 | | 94052 |
| 12:40367861_T_C | 28203, 94053- | 94090-94122 | 28263, 28266, |
| rs66737902_REF | 94089 | | 94123-94164 |
| 12:40367861_T_C | 94059, 94063, | 94096, 94098, | 94130, 94140, |
| rs66737902_SNP | 94067, 94070- | 94102, 94105- | 94143, 94146, |
| | 94071, 94080, | 94106, 94109, | 94148, 94152, |
| | 94082-94083, | 94115-94116, | 94155, 94157, |
| | 94086, 94165- | 94119, 94201- | 94160-94161, |
| | 94200 | 94236 | 94237-94278 |
| 12:40368129_A_C | 94279-94311 | 94312-94345 | 94346-94381 |
| rs10878441_REF | | | |
| 12:40368129_A_C | 94282, 94288, | 94312, 94316, | 94346-94347, |
| rs10878441_SNP | 94292, 94304, | 94322, 94327, | 94351, 94357, |
| | 94382-94416 | 94340, 94417- | 94375, 94450- |
| | | 94449 | 94484 |
| 12:40368149_C_T | 28531, 28536, | 28556, 28560, | 28577, 28580, |
| rs3886747_REF | 28539, 94282, | 94312, 94316, | 28585, 28587, |
| | 94288, 94292, | 94322, 94327, | 94346-94347, |
| | 94295, 94297, | 94330, 94333, | 94351, 94357, |
| | 94302, 94304, | 94337, 94340, | 94362, 94365, |
| | 94310, 94485- | 94342, 94344, | 94368, 94370, |
| | 94517 | 94518-94547 | 94372, 94375, |
| | | | 94377, 94380, |
| | | | 94548-94581 |
| 12:40368149_C_T | 94295, 94297, | 94330, 94333, | 94365, 94368, |
| rs3886747_SNP | 94302, 94310, | 94337, 94342, | 94370, 94377, |
| | 94488, 94490, | 94344, 94522, | 94380, 94551, |
| | 94498-94499, | 94530-94531, | 94556, 94561- |
| | 94510, 94582- | 94540-94541, | 94562, 94573, |
| | 94603 | 94604-94616 | 94617-94636 |
| 12:40368501_G_C | 28607-28608, | 28616, 28663, | 28621-28622, |
| rs1365770_REF | 28630, 28634, | 28666, 28672, | 28693, 28697, |
| | 28641, 28644, | 28675, 28683, | 28704, 28715, |
| | 28654, 94637- | 94665-94685 | 28721, 94686- |
| | 94664 | | 94709 |
| 12:40368501_G_C | 94637-94638, | 94665-94667, | 94686-94688, |
| rs1365770_SNP | 94640, 94643, | 94671, 94685, | 94694-94695, |
| | 94646, 94652, | 94738-94759 | 94760-94785 |
| | 94658, 94664, | | |
| | 94710-94737 | | |
| 12:40368690_CT_C | 28783, 94786- | 94798-94807 | 94808-94819 |
| rs367772598_REF | 94797 | | |
| 12:40368690_C_CTTT | 28783, 94786- | 94798-94807 | 94808-94819 |
| rs386376255_REF | 94797 | | |
| 12:40368744_T_C | 28925, 28945, | 94850-94877 | 94878-94911 |
| rs10784548_REF | 94820-94849 | | |
| 12:40368744_T_C | 94825, 94829, | 94851, 94855- | 94879, 94884- |
| rs10784548_SNP | 94835, 94839- | 94856, 94860, | 94885, 94889- |
| | 94840, 94846, | 94865, 94868- | 94891, 94900, |
| | 94912-94944 | 94869, 94945- | 94976-95010 |
| | | 94975 | |
| 12:40369805_C_T | 95011-95043 | 95044-95050 | 95051-95066 |
| rs1465527_REF | | | |
| 12:40369805_C_T | 95018, 95021, | 95045, 95050 | 95055, 95057, |
| rs1465527_SNP | 95023, 95033, | | 95063 |
| | 95037 | | |
| 12:40369814_C_T | 95011-95014, | 95046-95049, | 95051-95052, |
| rs1465528_REF | 95016-95017, | 95078-95082 | 95054, 95056, |
| | 95019-95020, | | 95058-95061, |
| | 95022, 95024- | | 95065-95066, |
| | 95025, 95027- | | 95083-95090 |
| | 95028, 95030- | | |
| | 95031, 95035- | | |
| | 95036, 95038- | | |
| | 95039, 95041- | | |
| | 95043, 95067- | | |
| | 95077 | | |
| 12:40369814_C_T | 95019, 95028, | 95078-95080, | 95084-95085, |
| rs1465528_SNP | 95039, 95070- | 95082 | 95087, 95089- |
| | 95072, 95074, | | 95091 |
| | 95076 | | |
| 12:40369850_G_T | 95092-95121 | 95122-95136 | 95137-95158 |
| rs17444285_REF | | | |
| 12:40369850_G_T | 95096, 95101, | 95124, 95127, | 95142, 95149, |
| rs17444285_SNP | 95106-95107, | 95131, 95133- | 95152-95153, |
| | 95110, 95112, | 95136 | 95155-95158, |
| | 95116-95117, | | 95162 |
| | 95120-95121, | | |
| | 95159-95161 | | |
| 12:40370171_C_G | 95163-95210 | 95211-95260 | 95261-95312 |
| rs7968048_REF | | | |
| 12:40370171_C_G | 95165, 95169- | 95213, 95217, | 95267, 95269, |
| rs7968048_SNP | 95171, 95180, | 95219, 95228, | 95277, 95279, |
| | 95182, 95191, | 95230, 95239, | 95281, 95294- |
| | 95195, 95206, | 95243-95244, | 95295, 95306- |
| | 95208, 95313- | 95255-95256, | 95307, 95312, |
| | 95350 | 95351-95390 | 95391-95432 |
| 12:40370967_T_G | 95433-95443 | , | 95444 |
| rs11609433_REF | | | |
| 12:40370967_T_G | 95433, 95438, | 95458-95468 | 95444, 95469- |
| rs11609433_SNP | 95445-95457 | | 95483 |
| 12:40371783_GAAAAAAAAA | 95484-95491 | 95492-95499 | 95500-95509 |
| G | | | |
| rs776238897_REF | | | |
| 12:40371783_GAAAA_G | 95484-95491 | 95492-95499 | 95500-95509 |
| rs149833872_REF | | | |
| 12:40371783_GA_G | 95484-95491 | 95492-95499 | 95500-95509 |
| rs1299545494_REF | | | |
| 12:40371801_A_C | 95510-95514 | 95515-95516 | 95517-95519 |
| rs4374003_REF | | | |
| 12:40371801_A_C | 95510-95514, | 95515-95516, | 95517-95519, |
| rs4374003_SNP | 95520-95522 | 95523-95525 | 95526-95530 |
| 12:40372127_A_G | 95531-95559 | 95560-95573 | 95574-95596 |
| rs7962370_REF | | | |
| 12:40372127_A_G | 95538, 95543, | 95563-95564, | 95578-95579, |
| rs7962370_SNP | 95548, 95551- | 95570-95572, | 95584, 95589, |
| | 95552, 95554- | 95635-95666 | 95591, 95594, |
| | 95555, 95597- | | 95667-95704 |
| | 95634 | | |
| 12:40224997-40225496 | 14, 21, 26, 31, | 67, 79, 84, 96, | 117, 129, 134, |
| Up to 500 bp Downstream | 46, 151, 153, | 244, 357-358, | 147-148, 268, |
| to Exon 51 | 168, 173, 193, | 361-363, 367, | 293, 295-296, |
| | 310, 313-314, | 370, 375, 381, | 397, 408-409, |
| | 318, 320-321, | 383, 499, 508, | 412, 414, 418, |
| | 326, 333, 343, | 512, 517, 520, | 420, 422, 433 |
| | 451, 454, 460, | 529-530, 535 | 435, 558, 562- |
| | 464, 469, 478, | 540, 542, 657 | 563, 568 571, |
| | 480-481, 486, | 659, 663, 666- | 580, 586, 590, |
| | 491, 609, 611, | 667, 670, 681, | 592, 594, 707- |
| | 615, 618-619, | 683, 685, 690, | 708, 710, 717- |
| | 622, 630, 633, | 96490-97286 | 718, 721, 732, |
| | 636, 642, | | 734, 740, 742, |
| | 95705-96489 | | 97287-98083 |
| 12:40225283-40225554 | 95705, 95707- | 96490, 96492- | 97287, 97289- |
| Intron 1 | 95709, 95711, | 96494, 96496, | 97291, 97293, |
| | 95713-95714, | 96498-96499, | 97295-97296, |
| | 95716-95720, | 96501-96505, | 97298-97302, |
| | 95722-95725, | 96507-96510, | 97304-97307, |
| | 95730, 95733, | 96515, 96518, | 97312, 97315, |
| | 95735, 95738, | 96520, 96523, | 97317, 97320, |
| | 95740, 95742- | 96525, 96527- | 97322, 97324- |
| | 95743, 95746- | 96528, 96531- | 97325, 97328- |
| | 95753, 95757, | 96538, 96542, | 97335, 97339, |
| | 95759-95763, | 96544-96548, | 97341-97345, |
| | 95771, 95776- | 96556, 96562- | 97353, 97358- |
| | 95777, 95781- | 96563, 96566- | 97359, 97362- |
| | 95782, 95785, | 96567, 96570, | 97363, 97366, |
| | 95792, 95795- | 96577, 96580, | 97373, 97376, |
| | 95798, 95800, | 96582-96584, | 97378-97380, |
| | 95802-95803, | 96586, 96588- | 97382, 97384- |
| | 95805, 95810- | 96589, 96591, | 97385, 97387, |
| | 95811, 95815, | 96594, 96597- | 97392-97393, |
| | 95824, 95826, | 96598, 96602, | 97397, 97406, |
| | 95831-95833, | 96611, 96614, | 97409, 97414- |
| | 95835-95838, | 96619-96621, | 97416, 97418- |
| | 95842, 95844- | 96623-96626, | 97421, 97426, |
| | 95845, 95847- | 96630, 96632- | 97428-97429, |
| | 95849, 95852, | 96633, 96635- | 97431-97433, |
| | 95854-95855, | 96637, 96640, | 97436, 97438- |
| | 95860, 95863- | 96642-96643, | 97439, 97445, |
| | 95864, 95867, | 96648, 96651- | 97448-97449, |
| | 95873, 95875, | 96652, 96655, | 97453, 97459, |
| | 95878, 95880- | 96661, 96663, | 97461, 97464, |
| | 95882, 95885- | 96666, 96668- | 97466-97468, |
| | 95891, 95893- | 96670, 96673- | 97471-97477, |
| | 95894, 95897, | 96679, 96681- | 97479-97480, |
| | 95906, 95909, | 96682, 96685, | 97483, 97492, |
| | 95916, 95918, | 96694, 96697, | 97495, 97505, |
| | 95920, 95924, | 96707, 96709, | 97507, 97509, |
| | 95927, 95933, | 96711, 96715, | 97513, 97516, |
| | 95943, 95947- | 96718, 96724, | 97522, 97532, |
| | 95948, 95950, | 96734, 96738- | 97536-97538, |
| | 95952, 95954, | 96740, 96742- | 97540-97542, |
| | 95956, 95958, | 96744, 96746, | 97544, 97547- |
| | 95961-95962, | 96749-96750, | 97548, 97550, |
| | 95964, 95967, | 96752, 96755- | 97554, 97556, |
| | 95970, 95972, | 96756, 96758, | 97558, 97561- |
| | 95975-95977, | 96761-96763, | 97563, 97566, |
| | 95979, 95983, | 96766, 96770, | 97570, 97574- |
| | 95987-95990, | 96774-96777, | 97577, 97579- |
| | 95992-95994, | 96779-96781, | 97581, 97586, |
| | 95999, 96009, | 96786, 96797, | 97596, 97600- |
| | 96013-96014, | 96801-96802, | 97601, 97603, |
| | 96017, 96024, | 96804, 96814, | 97613, 97617, |
| | 96027, 96030, | 96818, 96820, | 97619, 97622- |
| | 96032-96033, | 96823-96824, | 97623, 97625, |
| | 96035, 96037- | 96826, 96828- | 97627-97629, |
| | 96038, 96042, | 96830, 96834, | 97633, 97635- |
| | 96044-96045, | 96836-96837, | 97636, 97641, |
| | 96050, 96054- | 96842, 96846- | 97644, 97646, |
| | 96055, 96058, | 96847, 96851, | 97650, 97659- |
| | 96068-96072, | 96859, 96861- | 97663, 97665, |
| | 96074, 96078- | 96865, 96867, | 97668-97675, |
| | 96085, 96087, | 96871-96878, | 97677, 97679, |
| | 96089, 96091- | 96880, 96882, | 97681-97683, |
| | 96093, 96099, | 96884-96887, | 97689, 97693, |
| | 96102, 96104- | 96892, 96896, | 97695-97696, |
| | 96105, 96109- | 96898-96899, | 97700-97701, |
| | 96110, 96114- | 96903-96904, | 97705-97708, |
| | 96117, 96119- | 96908-96912, | 97710-97711, |
| | 96120, 96125, | 96914-96915, | 97718, 97720, |
| | 96127, 96130, | 96921, 96923, | 97723, 97725, |
| | 96132, 96134, | 96926, 96928, | 97727, 97730, |
| | 96137, 96141- | 96930, 96933, | 97734-97737, |
| | 96145, 96148, | 96937-96940, | 97741, 97743, |
| | 96151, 96153, | 96944, 96946, | 97745, 97752, |
| | 96160, 96162, | 96948, 96955, | 97754, 97765, |
| | 96173, 96178, | 96957, 96968, | 97770, 97773, |
| | 96183, 96186, | 96972, 96975, | 97776, 97780, |
| | 96188, 96196- | 96978, 96982, | 97787-97788, |
| | 96197, 96200, | 96989-96990, | 97791, 97794- |
| | 96203-96204, | 96993, 96995- | 97795, 97800- |
| | 96209-96212, | 96996, 97001- | 97803, 97805- |
| | 96214-96215, | 97004, 97006- | 97806, 97809, |
| | 96218, 96220- | 97007, 97010, | 97811-97812, |
| | 96221, 96225, | 97012-97013, | 97816, 97823, |
| | 96234, 96237- | 97017, 97023, | 97825-97826, |
| | 96238, 96240, | 97025-97027, | 97828, 97830- |
| | 96242-96243, | 97029-97030, | 97831, 97834- |
| | 96247-96248, | 97033-97035, | 97836, 97838, |
| | 96250, 96252, | 97037, 97039, | 97841, 97844, |
| | 96255, 96258, | 97042, 97044, | 97846, 97848- |
| | 96260, 96262- | 97046-97047, | 97849, 97857- |
| | 96263, 96271- | 97055-97057, | 97858, 97861- |
| | 96273, 96276- | 97060-97061, | 97862, 97867- |
| | 96277, 96283- | 97067-97068, | 97868, 97870- |
| | 96284, 96286- | 97070-97071, | 97871, 97873, |
| | 96287, 96289, | 97073, 97078, | 97878, 97890- |
| | 96304-96305, | 97091-97092, | 97891, 97893- |
| | 96307-96311, | 97094-97098, | 97897, 97903, |
| | 96316, 96318, | 97104, 97106, | 97905, 97907, |
| | 96320-96321, | 97108, 97110, | 97909, 97911- |
| | 96323-96325, | 97112-97114, | 97913, 97915, |
| | 96327, 96329, | 97116, 97118, | 97917, 97922- |
| | 96333-96334, | 97122-97123, | 97923, 97931- |
| | 96342-96343, | 97131-97132, | 97932, 97934, |
| | 96345, 96348- | 97134, 97138, | 97938, 97940- |
| | 96350, 96353, | 97140-97141, | 97941, 97944, |
| | 96355-96357, | 97144, 97147- | 97947-97949, |
| | 96359, 96363- | 97149, 97151, | 97951, 97956- |
| | 96365, 96367, | 97156-97158, | 97958, 97960, |
| | 96370-96371, | 97160, 97163- | 97963-97964, |
| | 96373-96374, | 97164, 97166- | 97966-97967, |
| | 96379, 96381- | 97167, 97172, | 97972, 97974, |
| | 96383, 96385, | 97174, 97176- | 97976-97977, |
| | 96390-96392, | 97177, 97179, | 97979, 97983- |
| | 96395, 96397- | 97183-97185, | 97985, 97988, |
| | 96398, 96403, | 97188, 97191- | 97991-97992, |
| | 96413, 96417, | 97192, 97197- | 97996-97997, |
| | 96420, 96425- | 97198, 97208, | 98006, 98010, |
| | 96427, 96429- | 97212, 97215, | 98013, 98018- |
| | 96430, 96432, | 97219-97221, | 98019, 98021- |
| | 96434, 96436, | 97223-97224, | 98022, 98024, |
| | 96441, 96443, | 97226, 97228, | 98026, 98029, |
| | 96446-96448, | 97231, 97236, | 98034, 98036, |
| | 96450-96452, | 97238, 97242- | 98040-98045, |
| | 96454-96457, | 97247, 97249- | 98047-98050, |
| | 96460-96461, | 97252, 97255, | 98053, 98055, |
| | 96463, 96465, | 97257, 97259, | 98057, 98059, |
| | 96467-96476, | 97261, 97263, | 98061, 98064- |
| | 96478-96480, | 97266-97273, | 98071, 98073- |
| | 96483-96489, | 97275-97277, | 98075, 98078- |
| | 98084-98199 | 97280-97286, | 98083, 98316- |
| | | 98200-98315 | 98431 |
| 12:40366506-40367005 | 98432-98857 | 98858-99333 | 99334-99715 |
| Intron 49 up to 500 bp | | | |
| Upstream to Exon 50 | | | |
| 12:40367078-40367643 | 93964-93967, | 93978-93987, | 93988-93991, |
| Intron 50 | 93969-93977, | 99872-100053 | 93993-93998, |
| | 99716-99871 | | 100054-100193 |
| 12:40225641-40226140 | 100194-100907 | 100908-101671 | 101672-102343 |
| Intron 2 up to 500 bp | | | |
| Downstream to Exon 2 | | | |
| 12:40235126-40235625 | 102344-102742 | 102743-103177 | 103178-103604 |
| Intron 3 up to 500 bp | | | |
| Upstream to Exon 4 | | | |

The indicated locations listed in column 1 of the Table 1 are based on gnomAD v3 database and UCSC Genome Browser assembly ID: hg38, Sequencing/Assembly provider ID: Genome Reference Consortium Human GRCh38.p12 (GCA_000001405.27). Assembly date: December 2013 initial release; December 2017 patch release 12.
The SNP details are indicated by the listed SNP ID Nos. (“rs numbers”), which are based on the NCBI 2018 database of Single Nucleotide Polymorphisms (dbSNP)). The indicated DNA mutations are associated with Transcript Consequence NM_198578 as obtained from NCBI RefSeq genes.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only.

### Experimental Details

### Example 1: LRRK2 On-Target Activity Anaylsis

Guide sequence portions comprising 17-50 contiguous nucleotides containing nucleotides in the sequence set forth in any one of SEQ ID NOs: 1-103,606 are screened for high on target activity using SpCas9 in HeLa cells. On target activity is determined by DNA capillary electrophoresis analysis.

### Example 2: LRRK2 Editing Strategy Analysis Using SpCas9

Point mutations in the leucine-rich repeat kinase 2 (LRRK2) gene are the most frequent cause of familial, autosomal dominant Parkinson's disease. In order to develop an editing strategy requiring only one guide RNA molecule to target and inactivate an LRRK2 mutant allele, guide RNA molecules having guide sequence portions which target SNPs residing in the coding region of the gene were screened. Specifically, guides having one of nine (9) different guide sequence portions, each of which target a CRISPR complex to one of four different SNP positions (rs1878245, rs1427263, rs1878405, or rs3761863), were screened in A549 cells that express LRRK2 to determine if the guides could lead to the formation of indels and/or frameshift mutations in the LRRK2 coding sequence that could result in expression of a truncated protein or induce nonsense mediated decay (NMD).

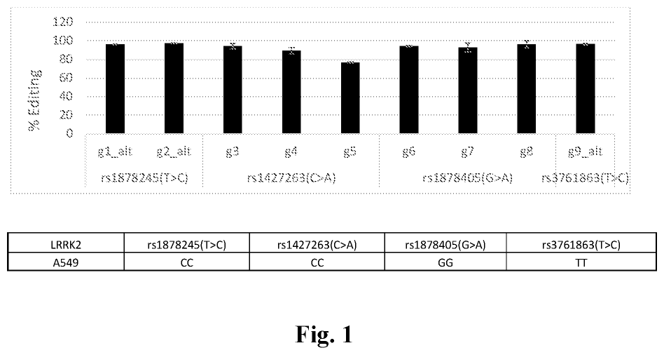

Briefly, pre-assembled RNPs composed of 105 pmole SpCas9 CRISPR nuclease protein and 120 pmole of a 20-nucleotide sgRNA (Table 2) were mixed with 100 pmole of electroporation enhancer (IDT-1075916) and electroporated into 2×10⁵ A549 cells using the SF primary cell 4D-nucleofector X Kit S (PBC2-00675, Lonza) and applying the CM-130 program. A fraction of cells was harvested 72 hours post electroporation, genomic DNA was extracted, and amplicons generated from the extracted DNA was subjected to next-generation sequencing (NGS) analysis. As shown in the results presented in FIG. 1, the tested guide molecules depicted high editing activity at the site of the targeted SNPs, ranging from 76%-97% on-target activity.

To assess the effect of the editing on the level of LRRK2 expression, total RNA was extracted from A549 cells seven (7) days post-electroporation and the level of LRRK2 mRNA was measured by qRT-PCR. A decrease in the level of LRRK2 mRNA was detected only for guide molecules “g8” and “g9_alt,” measuring 60% and 40% editing respectively. The editing pattern for the rest of the guide molecules indicates that truncated protein expression could be produced as the result of a frameshift mutation.

TABLE 2
Sequences of 20-nucleotide guide sequence
portions that target the human LRRK2
coding sequence.

| Location | SNP | Guide | Sequence | PAM |
|---|---|---|---|---|
| Exon 5 | rs1878245 | g1_alt | UUCUUCAUCCAGUAUCAGCA | AGG |
| | (T > C) | | (SEQ ID NO: 33653) | |
| | | g2_alt | UAAAAUCACCUUGCUGAUAC | UGG |
| | | | (SEQ ID NO: 33642) | |
| Exon 34 | rs1427263 | g3 | CUACGCGAAAUAAUGCCCUU | AGG |
| | (C > A) | | (SEQ ID NO: 61673) | |
| | | g4 | UACGCGAAAUAAUGCCCUUA | GGG |
| | | | (SEQ ID NO: 61681) | |
| | | g5 | AUAAUGCCCUUAGGGUGUUU | UGG |
| | | | (SEQ ID NO: 61661) | |
| Exon 43 | rs1878405 | g6 | AAUUUCUCAACCAUAGGCCA | UGG |
| | (G > A) | | (SEQ ID NO: 80365) | |
| | | g7 | AUUUCUCAACCAUAGGCCAU | GGG |
| | | | (SEQ ID NO: 80368) | |
| | | g8 | UUUCUCAACCAUAGGCCAUG | GGG |
| | | | (SEQ ID NO: 80384) | |
| Exon 49 | rs3761863 | g9_alt | GGUAACGGUAAAAGAAAACA | AGG |
| | (T > C) | | (SEQ ID NO: 93060) | |

### Example 3: LRRK2 Editing Strategy Analysis Using Alternate CRISPR Nucleases

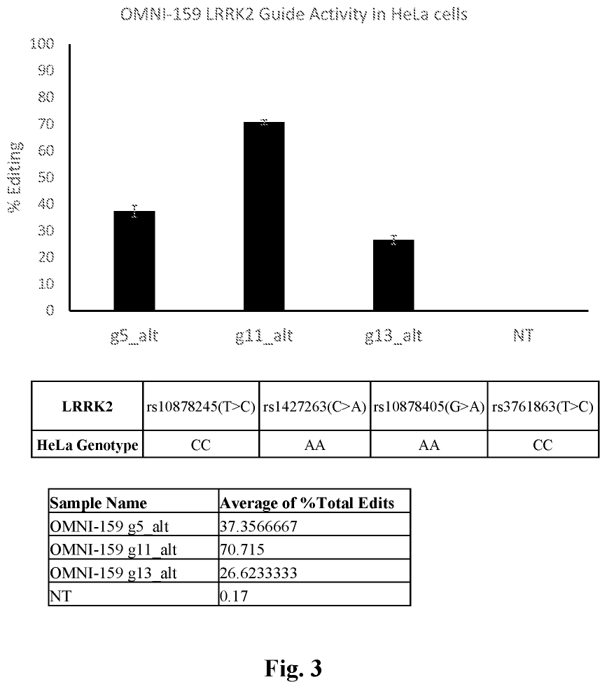

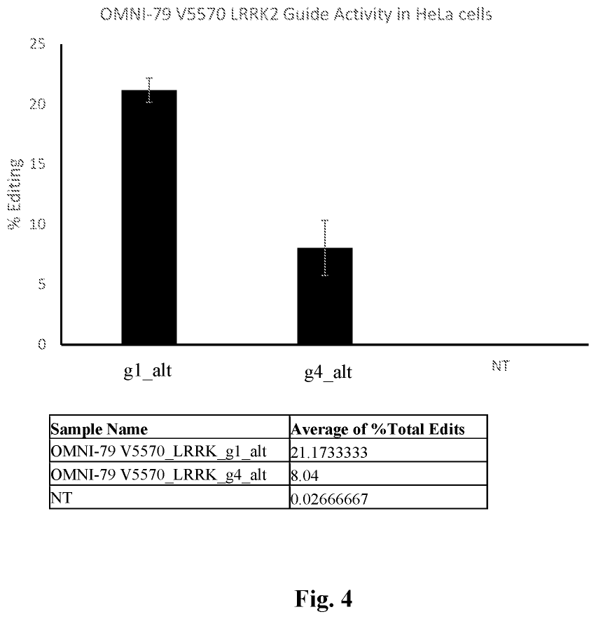

The editing activities of two alternate CRISPR nucleases, OMNI-159 (SEQ ID NO: 103612) and OMNI-79 V5570 (SEQ ID NO: 103613), were preliminarily assessed with select guides in HeLa cells (FIG. 3 and FIG. 4). These initial data suggest that the LRRK2-targeting guide sequence portions provided herein can be utilized with a variety of different CRISPR nucleases.

TABLE 3
Sequences of guide sequence portions that target the human
LRRK2 coding sequence used with alternate CRISPR nucleases.

| | | | | OMNI CRISPR |
|---|---|---|---|---|
| SNP Targeted | Guide Name | Guide Sequence Portion | PAM | Nuclease |
| rs3761863(T > C) | g11_alt | UCUAGGGAGGUAACGGUAAAGG | AAAACA | OMNI-159 |
| | | (SEQ ID NO: 93150) | | |
| rs1427263(C > A) | g5_alt | AAAUAAUUCCCUUAGGGUGUUU | TGGACA | OMNI-159 |
| | | (SEQ ID NO: 61854) | | |
| rs10878245(T > C) | g13_alt | AUAUCACUUUCUUCAUCCAGUA | TCAGCA | OMNI-159 |
| | | (SEQ ID NO: 33706) | | |
| rs10878245(T > C) | g1_alt | CUUUCUUCAUCCAGUAUCAGCA | AGGTG | OMNI-79 V5570 |
| | | (SEQ ID NO: 33719) | | |
| rs1427263(C > A) | g4_alt | UCUACGCGAAAUAAUUCCCUUA | GGGTG | OMNI-79 V5570 |
| | | (SEQ ID NO: 61889) | | |

### REFERENCES

1. Ahmad and Allen (1992) “Antibody-mediated Specific Binging and Cytotoxicity of Lipsome-entrapped Doxorubicin to Lung Cancer Cells in Vitro”, Cancer Research 52:4817-20.
2. Anderson (1992) “Human gene therapy”, Science 256:808-13.
3. Basha et al. (2011) “Influence of Cationic Lipid Composition on Gene Silencing Properties of Lipid Nanoparticle Formulations of siRNA in Antigen-Presenting Cells”, Mol. Ther. 19(12):2186-200.
4. Behr (1994) Gene transfer with synthetic cationic amphiphiles: Prospects for gene therapy”, Bioconjuage Chem 5:382-89.

Blaese (1995) “Vectors in cancer therapy: how will they deliver”, Cancer Gene Ther.

2:291-97.
6. Blaese et al. (1995) “T lympocyte-directed gene therapy for ADA-SCID: initial trial results after 4 years”, Science 270(5235):475-80.
7. Buchschacher and Panganiban (1992) “Human immunodeficiency virus vectors for inducible expression of foreign genes”, J. Virol. 66:2731-39.
8. Burstein et al. (2017) “New CRISPR-Cas systems from uncultivated microbes”, Nature 542:237-41.
9. Chang and Wilson (1987) “Modification of DNA ends can decrease end-joining relative to homologous recombination in mammalian cells”, Proc. Natl. Acad. Sci. USA 84:4959-4963.
10. Chung et al. (2006) “Agrobacterium is not alone: gene transfer to plants by viruses and other bacteria”, Trends Plant Sci. 11(1):1-4.
11. Coelho et al. (2013) “Safety and efficacy of RNAi therapy for transthyretin amyloidosis” N. Engl. J. Med. 369, 819-829.
12. Collias and Beisel (2021) “CRISPR technologies and the search for the PAM-free nuclease”, Nature Communications 12, 555.
13. Crystal (1995) “Transfer of genes to humans: early lessons and obstacles to success”, Science 270(5235):404-10.
14. Dillon (1993) “Regulation gene expression in gene therapy” Trends in Biotechnology 11(5):167-173.
15. Dranoff et al. (1997) “A phase I study of vaccination with autologous, irradiated melanoma cells engineered to secrete human granulocyte macrophage colony stimulating factor”, Hum. Gene Ther. 8(1):111-23.
16. Dunbar et al. (1995) “Retrovirally marked CD34-enriched peripheral blood and bone marrow cells contribute to long-term engraftment after autologous transplantation”, Blood 85:3048-57.
17. Ellem et al. (1997) “A case report: immune responses and clinical course of the first human use of ganulocyte/macrophage-colony-stimulating-factor-tranduced autologous melanoma cells for immunotherapy”, Cancer Immunol Immunother 44:10-20.
18. Gao and Huang (1995) “Cationic liposome-mediated gene transfer” Gene Ther. 2(10):710-22.
19. Haddada et al. (1995) “Gene Therapy Using Adenovirus Vectors”, in: The Molecular Repertoire of Adenoviruses III: Biology and Pathogenesis, ed. Doerfler and Bohm, pp. 297-306.
20. Han et al. (1995) “Ligand-directed retro-viral targeting of human breast cancer cells”, Proc Natl Acad Sci U.S.A. 92(21):9747-51.
21. Hu et al. (2018) “Evolved Cas9 variants with broad PAM compatibility and high DNA specificity”, Nature 556(7699):57-63.
22. Inaba et al. (1992) “Generation of large numbers of dendritic cells from mouse bone marrow cultures supplemented with granulocyte/macrophage colony-stimulating factor”, J Exp Med. 176(6):1693-702.
23. Jinek et al. (2012) “A programmable dual-RNA-guided DNA endonuclease in adaptive bacterial immunity,” Science 337(6096):816-21.
24. Johan et al. (1992) “GLVR1, a receptor for gibbon ape leukemia virus, is homologous to a phosphate permease of Neurospora crassa and is expressed at high levels in the brain and thymus”, J Virol 66(3):1635-40.
25. Judge et al. (2006) “Design of noninflammatory synthetic siRNA mediating potent gene silencing in vivo”, Mol Ther. 13(3):494-505.
26. Kohn et al. (1995) “Engraftment of gene-modified umbilical cord blood cells in neonates with adnosine deaminase deficiency”, Nature Medicine 1:1017-23.
27. Kremer and Perricaudet (1995) “Adenovirus and adeno-associated virus mediated gene transfer”, Br. Med. Bull. 51(1):31-44.
28. Macdiarmid et al. (2009) “Sequential treatment of drug-resistant tumors with targeted minicells containing siRNA or a cytotoxic drug”, Nat Biotehcnol. 27(7):643-51.
29. Malech et al. (1997) “Prolonged production of NADPH oxidase-corrected granulocyes after gene therapy of chronic granulomatous disease”, PNAS 94(22):12133-38.
30. Miller et al. (1991) “Construction and properties of retrovirus packaging cells based on gibbon ape leukemia virus”, J Virol. 65(5):2220-24.
31. Miller (1992) “Human gene therapy comes of age”, Nature 357:455-60.
32. Mitani and Caskey (1993) “Delivering therapeutic genes—matching approach and application”, Trends in Biotechnology 11(5):162-66.
33. Nabel and Felgner (1993) “Direct gene transfer for immunotherapy and immunization”, Trends in Biotechnology 11(5):211-15.
34. Nishimasu et al. (2018) “Engineered CRISPR-Cas9 nuclease with expanded targeting space”, Science 361(6408):1259-1262.
35. Remy et al. (1994) “Gene Transfer with a Series of Lipphilic DNA-Binding Molecules”, Bioconjugate Chem. 5(6):647-54.
36. Sentmanat et al. (2018) “A Survey of Validation Strategies for CRISPR-Cas9 Editing”, Scientific Reports 8:888, doi:10.1038/s41598-018-19441-8.
37. Sommerfelt et al. (1990) “Localization of the receptor gene for type D simian retroviruses on human chromosome 19”, J. Virol. 64(12):6214-20.
38. Van Brunt (1988) “Molecular framing: transgenic animals as bioactors” Biotechnology 6:1149-54.
39. Vigne et al. (1995) “Third-generation adenovectors for gene therapy”, Restorative Neurology and Neuroscience 8(1,2): 35-36.
40. Walton et al. (2020) “Unconstrained genome targeting with near-PAMless engineered CRISPR-Cas9 variants”, Science 368(6488):290-296.
41. Weissman and Kariko (2015) “mRNA:Fulfilling the promise of gene therapy”, Molecular Therapy (9):1416-7.
42. Wilson et al. (1989) “Formation of infectious hybrid virion with gibbon ape leukemia virus and human T-cell leukemia virus retroviral envelope glycoproteins and the gag and pol proteins of Moloney murine leukemia virus”, J. Virol. 63:2374-78.
43. Yu et al. (1994) “Progress towards gene therapy for HIV infection”, Gene Ther. 1(1):13-26.
44. Zetsche et al. (2015) “Cpf1 is a single RNA-guided endonuclease of a class 2 CRIPSR-Cas system” Cell 163(3):759-71.
45. Zuris et al. (2015) “Cationic lipid-mediated delivery of proteins enables efficient protein based genome editing in vitro and in vivo” Nat Biotechnol. 33(1):73-80.

1. A method for modifying in a cell a mutant allele of the Leucine-rich repeat serine/threonine-protein kinase 2 (LRRK2) gene having a mutation associated with monogenic Parkinson's disease, the method comprising
introducing to the cell a composition comprising:
at least one CRISPR nuclease or a sequence encoding a CRISPR nuclease; and
a first RNA molecule comprising a guide sequence portion having 17-50 nucleotides or a nucleotide sequence encoding the same,
wherein a complex of the CRISPR nuclease and the first RNA molecule affects a double strand break in the mutant allele of the LRRK2 gene.

2. The method of claim 1, wherein the first RNA molecule targets the CRISPR nuclease to the mutation associated with monogenic Parkinson's disease,
wherein the mutation associated with monogenic Parkinson's disease is any one of 12:40225041_C_T, 12:40225058_C_A, 12:40225088_G_A, 12:40225159_G_A, 12:40225233_G_C, 12:40225558_C_T, 12:40225618_T_C, 12:40225628_G_A, 12:40225631_T_C, 12:40232303_A_C, 12:40232345_G_C, 12:40232380_A_C, 12:40232385_T_C, 12:40235634_T_C, 12:40235646_C_G, 12:40235660_A_G, 12:40238078_A_G, 12:40240517_C_T, 12:40240531_T_C, 12:40240594_G_C, 12:40240602_T_C, 12:40243551_CAAT_C, 12:40243556_A_T, 12:40249822_T_G, 12:40249843_C_G, 12:40249854_C_T, 12:40249881_T_C, 12:40249908_A_G, 12:40251273_G_A, 12:40251303_G_A, 12:40251361_A_G, 12:40251366_C_A, 12:40251369_G_A, 12:40251495_C_T, 12:40252906_A_G, 12:40252929_G_A, 12:40252939_C_T, 12:40252952_T_G, 12:40252958_A_C, 12:40252968_G_A, 12:40252974_C_T, 12:40252978_C_T, 12:40252984_C_T, 12:40252988_T_C, 12:40253016_G_A, 12:40257311_A_G, 12:40257341_G_T, 12:40259507_A_G, 12:40259525_A_T, 12:40259534_G_T, 12:40259578_G_A, 12:40263806_A_G, 12:40263875_A_G, 12:40263900_G_A, 12:40263907_T_C, 12:40274646_CC_C, 12:40274881_T_C, 12:40274899_A_G, 12:40274917_G_C, 12:40277933_T_C, 12:40277968_A_C, 12:40277971_A_G, 12:40278025_T_A, 12:40278154_A_G, 12:40278167_C_T, 12:40283881_G_A, 12:40283915_T_C, 12:40283947_C_T, 12:40283966_G_A, 12:40283985_C_A, 12:40283990_T_C, 12:40284061_A_G, 12:40284076_C_A, 12:40284139_T_C, 12:40287363_C_A, 12:40287438_A_G, 12:40287461_A_G, 12:40293555_CTC_CCT, 12:40293607_C_T, 12:40293613_G_A, 12:40293624_G_C, 12:40293626_G_A, 12:40293644_A_G, 12:40294854_T_G, 12:40294862_T_C, 12:40294866_G_T, 12:40294878_C_T, 12:40295448_A_G, 12:40295463_A_G, 12:40295466_G_A, 12:40295535_A_T, 12:40295566_A_G, 12:40295574_A_C, 12:40295581_G_T, 12:40298330_A_G, 12:40298333_C_T, 12:40298346_G_A, 12:40298433_C_G, 12:40298479_G_T, 12:40298488_A_G, 12:40299125_A_G, 12:40299190_C_T, 12:40299196_A_C, 12:40299212_G_A, 12:40299255_T_C, 12:40302866_A_G, 12:40303952_C_A, 12:40303972_C_G, 12:40304000_G_A, 12:40304004_A_G, 12:40304035_A_C, 12:40304040_G_C, 12:40304040_G_T, 12:40304078_G_A, 12:40304141_C_T, 12:40305776_C_T, 12:40305781_T_A, 12:40305791_C_G, 12:40305871_G_A, 12:40305942_G_A, 12:40305946_T_A, 12:40308467_G_T, 12:40308476_A_G, 12:40308481_G_A, 12:40308543_GG_G, 12:40308632_C_A, 12:40308672_G_A, 12:40309174_G_A, 12:40309206_C_T, 12:40309225_A_C, 12:40309226_A_G, 12:40309230_A_G, 12:40309236_G_A, 12:40310434_C_A, 12:40310434_C_G, 12:40310434_C_T, 12:40310435_G_A, 12:40310436_C_T, 12:40310437_G_C, 12:40310440_T_G, 12:40310450_C_T, 12:40310461_G_A, 12:40310471_A_G, 12:40310476_GAT_G, 12:40310515_A_G, 12:40310530_C_T, 12:40310546_G_A, 12:40310554_G_A, 12:40310561_G_A, 12:40314043_G_A, 12:40314059_C_T, 12:40314062_A_G, 12:40314101_C_A, 12:40315266_T_A, 12:40319979_A_T, 12:40319998_T_C, 12:40320043_G_A, 12:40320054_G_C, 12:40320090_A_G, 12:40320094_A_G, 12:40320097_T_C, 12:40320119_A_G, 12:40321114_A_G, 12:40321123_T_C, 12:40322037_C_T, 12:40322038_G_A, 12:40322047_G_A, 12:40322047_G_T, 12:40322134_A_G, 12:40322137_A_C, 12:40322364_T_C, 12:40322386_G_T, 12:40322468_C_A, 12:40323244_C_G, 12:40323255_A_G, 12:40323256_T_C, 12:40323260_G_T, 12:40323270_G_T, 12:40323297_T_C, 12:40328416_G_C, 12:40335008_A_T, 12:40335031_G_A, 12:40335036_C_T, 12:40335037_G_A, 12:40335079_G_T, 12:40340349_C_G, 12:40340361_T_C, 12:40340380_T_C, 12:40340400_G_A, 12:40340404_T_C, 12:40340432_A_G, 12:40340436_A_T, 12:40346818_T_C, 12:40346826_ACTACT_A, 12:40346884_A_G, 12:40348402_G_GTGT, 12:40348475_A_G, 12:40348484_C_T, 12:40351537_A_G, 12:40351572_T_A, 12:40351579_C_T, 12:40351585_G_A, 12:40351605_G_C, 12:40351623_G_T, 12:40351667_C_A, 12:40351680_G_C, 12:40351720_G_C, 12:40351723_A_G, 12:40354304_T_C, 12:40354305_G_T, 12:40354410_G_A, 12:40356126_A_T, 12:40356145_C_T, 12:40359345_C_T, 12:40359422_A_G, 12:40363435_C_T, 12:40363440_C_T, 12:40363541_G_A, 12:40364843_G_A, 12:40364845_G_GGT, 12:40364884_G_A, 12:40364927_A_T, 12:40364960_A_G, 12:40365044_C_T, 12:40367012_T_A, 12:40367050_A_G, 12:40367648_AC_A, 12:40367656_G_T, 12:40367754_T_C, 12:40367849_G_A, 12:40367888_A_C, 12:40367988_C_T, 12:40368042_A_G, 12:40368073_A_C, 12:40368170_C_T, 12:40368262_C_T, 12:40368380_T_A, 12:40368486_G_T, 12:40368516_C_A, 12:40368534_T_G, 12:40368673_A_C, 12:40368736_C_A, 12:40368783_G_T, 12:40368800_T_C, 12:40368807_A_G, 12:40368834_A_G, 12:40368860_C_T, 12:40368905_A_T, 12:40368930_C_T, 12:40368947_C_T, 12:40368951_C_T, 12:40369044_T_C, 12:40369153_T_C, and 12:40369167_G_T; and/or
wherein the guide sequence portion of the first RNA molecule comprises 17-50 contiguous nucleotides containing nucleotides in the sequence set forth in any one of SEQ ID NOs: 1-103,606 that targets a mutation associated with monogenic Parkinson's disease.

3-4. (canceled)

5. The method of claim 1, wherein the first RNA molecule targets the CRISPR nuclease to a SNP position of the mutant allele,
wherein the SNP position is any one of rs1388598, rs28365207, rs7294619, rs10878226, rs1491943, rs1491942, rs1491941, rs11175645, rs4567538, rs12230685, rs11175655, rs11175656, rs1402299410, rs780970090, rs1479481931, rs71078229, rs11175658, rs3057613, rs35642658, rs74324737, rs2708441, rs10631840, rs35991926, rs61931634, rs11175666, rs751672176, rs36024911, rs377568728, rs1279923177, rs2249281, rs10878244, rs138000936, rs10878245, rs10878246, rs10878247, rs954884, rs10878249, rs10784444, rs6581622, rs17519573, rs35987733, rs868476079, rs1399756142, rs1474763022, rs1450276224, rs1191097078, rs1425272990, rs1477924701, rs2404580, rs2404581, rs1388599, rs1388596, rs10878258, rs12371502, rs11175729, rs17443414, rs1486599371, rs7134379, rs7955902, rs1491939, rs1491938, rs7969677, rs2723265, rs7132171, rs10784451, rs10784452, rs1907633, rs1491936, rs1907632, rs2046928, rs2723264, rs17465870, rs10878278, rs11175766, rs11564183, rs11175769, rs17465912, rs17443552, rs17490817, rs11430638, rs4293189, rs10784461, rs7308720, rs10784462, rs10878284, rs11175780, rs11175784, rs11175787, rs144463374, rs73108329, rs12426891, rs4768224, rs11175793, rs10784470, rs12146857, rs11564207, rs7308193, rs10506150, rs76660489, rs10878299, rs373619252, rs10878302, rs11564129, rs7971935, rs11175836, rs4272849, rs10784485, rs10506151, rs7309197, rs7134572, rs11175843, rs147801391, rs376096297, rs7488873, rs11175845, rs7979341, rs1226327150, rs777452337, rs1339001348, rs11175847, rs112859457, rs17466080, rs11175852, rs10784486, rs73108368, rs398019201, rs17519846, rs17466150, rs11175862, rs4310676, rs11564150, rs4318033, rs4473003, rs1429478461, rs886049356, rs11564270, rs17519881, rs17466178, rs17466185, rs11175902, rs76033741, rs56325408, rs7957754, rs11175911, rs12369882, rs11175913, rs4483664, rs66487867, rs1491426750, rs5006481, rs139565575, rs150276517, rs72446556, rs1186032972, rs12820920, rs7966550, rs12314692, rs11175922, rs73102746, rs10784497, rs17443815, rs7957057, rs76607433, rs28365224, rs7962011, rs10878336, rs11175934, rs11175935, rs17491061, rs11175941, rs10878340, rs10784498, rs10878341, rs10784499, rs17484286, rs146071397, rs10878343, rs11564180, rs11175958, rs11175959, rs113772607, rs11564149, rs11175963, rs7305344, rs7133914, rs11175964, rs397805328, rs11175966, rs10784501, rs41286474, rs869212703, rs2069228, rs10784503, rs386376250, rs11564128, rs2896975, rs7302503, rs7137160, rs369794148, rs34709132, rs10467144, rs11175985, rs10784504, rs57421387, rs7973058, rs1427267, rs1427266, rs721713, rs721712, rs721711, rs721709, rs4768227, rs398098159, rs4767969, rs10878356, rs10784506, rs10878358, rs7132073, rs1427265, rs17466339, rs2114569, rs2114568, rs2162469, rs10784509, rs1896254, rs5797667, rs373676201, rs1896253, rs1896252, rs1427263, rs11176013, rs11564148, rs11564205, rs10748030, rs10748031, rs10748032, rs11564177, rs10748033, rs10735931, rs10748034, rs10878368, rs7307276, rs4768229, rs4768230, rs397968969, rs11564204, rs4423250, rs7137665, rs10878371, rs11176022, rs60750674, rs10878372, rs398055492, rs761931834, rs1415940047, rs17466430, rs11176030, rs3747891, rs7298930, rs17444089, rs10878377, rs12316801, rs17444096, rs11564175, rs2404832, rs17466486, rs11564267, rs17520244, rs10506152, rs11176052, rs11176053, rs17444124, rs11564203, rs7307562, rs17520251, rs11461395, rs10506153, rs73275761, rs963243, rs17444145, rs10878386, rs17466521, rs12367542, rs17520278, rs35031086, rs11834950, rs11829088, rs4465428, rs61007767, rs60776971, rs2404834, rs7302841, rs2404835, rs11356084, rs715403, rs715402, rs6581667, rs6581668, rs200521371, rs4767970, rs4767971, rs4767972, rs1427271, rs1427272, rs1427273, rs10735934, rs10732751, rs12306060, rs1365763, rs7963697, rs11316380, rs7963086, rs17491536, rs7956787, rs7956898, rs10506155, rs7954061, rs7957151, rs919174, rs3065921, rs919175, rs1035812, rs1365764, rs397724721, rs17461964, rs10784518, rs4768231, rs4768232, rs3943893, rs2162471, rs1035813, rs10459264, rs10459265, rs11564147, rs10715759, rs7296657, rs7312497, rs10715758, rs10784522, rs796827140, rs74467833, rs7131694, rs12311273, rs58911468, rs35847030, rs1365765, rs66513412, rs2896977, rs199606989, rs1365766, rs10878405, rs11176143, rs7294958, rs61915610, rs7294952, rs59096461, rs59980086, rs4768233, rs4768234, rs4768235, rs7314455, rs7313525, rs7313895, rs7314863, rs7303525, rs7132187, rs7307310, rs11176153, rs11176154, rs7311247, rs7137437, rs75439407, rs73107108, rs1307806388, rs10878409, rs10878410, rs138382676, rs11176161, rs10878412, rs10878413, rs183544696, rs189538849, rs11610569, rs142726158, rs1194726090, rs576444406, rs185723137, rs1179215763, rs71449759, rs61915613, rs140722234, rs191099092, rs113693842, rs112767992, rs34531686, rs12368227, rs12368229, rs3930031, rs7308626, rs890575, rs890575, rs10748040, rs61230125, rs11176165, rs7976715, rs7976724, rs7963987, rs2162472, rs767501990, rs552326490, rs1365381164, rs760911090, rs754028820, rs11564173, rs7306545, rs7306684, rs112259589, rs73277531, rs77689380, rs73277533, rs17462055, rs758880647, rs10784528, rs796949072, rs10784532, rs145580704, rs386376254, rs11836288, rs11564146, rs10784536, rs11277474, rs7971919, rs7962116, rs67512688, rs2896978, rs2404836, rs11176195, rs4767973, rs17444202, rs4768236, rs4768237, rs58724345, rs397897325, rs33962975, rs7134408, rs11835105, rs137858615, rs10878434, rs35403247, rs3761863, rs17444223, rs12426362, rs17466605, rs3789328, rs12426498, rs4768238, rs66737902, rs10878441, rs3886747, rs1365770, rs367772598, rs386376255, rs10784548, rs1465527, rs1465528, rs17444285, rs7968048, rs11609433, rs776238897, rs149833872, rs1299545494, rs4374003, and rs7962370;
wherein the guide sequence portion of the first RNA molecule comprises 17-50 contiguous nucleotides containing nucleotides in the sequence set forth in any one of SEQ ID NOs: 1-103,606 that targets a SNP position of the mutant allele;
wherein the SNP position is in an exon or intron of the LRRK2 mutant allele, or within 3,000 basepairs upstream of the transcription start site or within 3,000 basepairs downstream of the 3′ UTR of the LRRK2 mutant allele; and/or
wherein the SNP position contains a heterozygous SNP.

6-9. (canceled)

10. The method of claim 1, further comprising introducing to the cell a second RNA molecule comprising a guide sequence portion having 17-50 nucleotides or a nucleotide sequence encoding the same, wherein a complex of the second RNA molecule and a CRISPR nuclease affects a second double strand break in the LRRK2 gene.

11. The method of claim 10, wherein the guide sequence portion of the second RNA molecule comprises 17-50 contiguous nucleotides containing nucleotides in the sequence set forth in any one of SEQ ID NOs: 1-103,606 other than the sequence of the first RNA molecule,
wherein the second RNA molecule comprises a non-discriminatory guide portion that targets both functional and mutated LRRK2 alleles;
wherein the second RNA molecule comprises a non-discriminatory guide portion that targets any one of Intron 1 of LRRK2, Intron 49 of LRRK2, Intron 2 of LRRK2, Intron 3 of LRRK2, a 3′ untranslated region (3′ UTR) of LRRK2, and an intergenic region downstream of LRRK2;
wherein the second RNA molecule comprises a non-discriminatory guide portion that targets a sequence that is located within a genomic range selected from any one of 12:40224997-40225496, 12:40225283-40225554, 12:40366506-40367005, 12:40367078-40367643, 12:40225641-40226140, and 12:40235126-40235625; and/or
wherein the second RNA molecule comprises a non-discriminatory guide portion that targets a sequence that is located up to 500 base pairs from an exon that is excised by the first and second RNA molecules.

12-15. (canceled)

16. The method of claim 10, wherein an exon or a portion thereof is excised from the mutant allele of the LRRK2 gene.

17. The method of claim 1, wherein the guide sequence portion of the first RNA molecule comprises 1, 2, 3, 4, or 5 nucleotide mismatches relative to a fully complementary target sequence of the guide sequence portion; or
wherein the guide sequence portion comprises 17-50 contiguous nucleotides containing nucleotides in the sequence set forth in any one of SEQ ID NOs: 1-103,606 modified to contain 1, 2, 3, 4, or 5 nucleotide mismatches relative to a fully complementary target sequence of the guide sequence portion.

18. (canceled)

19. The method of claim 17, wherein the guide sequence portion provides higher targeting specificity to the complex of the CRISPR nuclease and the first RNA molecule relative to a guide sequence portion that has higher complementarity to the mutant allele of the LRRK2 gene.

19. The method of claim 17, wherein the guide sequence portion provides higher targeting specificity to the complex of the CRISPR nuclease and the first RNA molecule relative to a guide sequence portion that has higher complementarity to the mutant allele of the LRRK2 gene.

20. A modified cell obtained by the method of claim 1.

21. The modified cell of claim 20, wherein the modified cell is a stem cell, a neuron, or a dopaminergic cell.

22. A first RNA molecule comprising a guide sequence portion having 17-50 contiguous nucleotides containing nucleotides in the sequence set forth in any one of SEQ ID NOs: 1-103,606, or any one of SEQ ID NOs: 1-103,606 modified to contain 1, 2, 3, 4, or 5 nucleotide mismatches relative to a fully complementary target sequence of the guide sequence portion.

23. A composition comprising the first RNA molecule of claim 22 and at least one CRISPR nuclease.

24. The composition of claim 23, further comprising a second RNA molecule comprising a guide sequence portion having 17-50 contiguous nucleotides, wherein the second RNA molecule targets a LRRK2 allele, and wherein the guide sequence portion of the second RNA molecule is a different sequence from the sequence of the guide sequence portion of the first RNA molecule.

25. The composition of claim 24, wherein the guide sequence portion of the second RNA molecule comprises 17-50 contiguous nucleotides containing nucleotides in the sequence set forth in any one of SEQ ID NOs: 1-103,606, or any one of SEQ ID NOs: 1-103,606 modified to contain 1, 2, 3, 4, or 5 nucleotide mismatches relative to a fully complementary target sequence of the guide sequence portion.

26. A method for inactivating a mutant LRRK2 allele in a cell, the method comprising delivering to the cell the composition of claim 23.

27. A method for treating monogenic Parkinson's disease, the method comprising delivering to a cell of a subject having monogenic Parkinson's disease the composition of claim 23.

28. A method for inactivating a mutant LRRK2 allele in a cell, comprising delivering to the cell the composition of claim 20.

29. A medicament comprising the composition of claim 23 for use in inactivating a mutant LRRK2 allele in a cell, wherein the medicament is administered by delivering to the cell the composition of claim 23.

30. A method for treating ameliorating or preventing monogenic Parkinson's disease, comprising delivering to a cell of a subject having or at risk of having monogenic Parkinson's disease the composition of claim 23.

31. A medicament comprising the composition of claim 23 for use in treating ameliorating or preventing monogenic Parkinson's disease, wherein the medicament is administered by delivering to a cell of a subject having or at risk of having monogenic Parkinson's disease the composition of claim 23.
